(19) United States
(12) Patent Application Publication
(10) Pub. No.: US 2025/0354142 A1
(43) Pub. Date: Nov. 20, 2025

(54) FUNCTIONAL NUCLEIC ACID MOLECULE

(71) Applicant: FONDAZIONE ISTITUTO ITALIANO DI TECNOLOGIA, Genova (IT)

(72) Inventors: Stefano Gustincich, Genova (IT); Stefano Espinoza, Genova (IT)

(21) Appl. No.: 18/867,643

(22) PCT Filed: May 26, 2023
(86) PCT No.: PCT/EP2023/064210

§ 371 Date: Nov. 20, 2024

(30) Foreign Application Priority Data
May 26, 2022 (IT) 2207796.0

(51) Int. Cl. C12N 15/113

(52) U.S. Cl. CPC C12N 15/113; C12N 2310/11

(57) ABSTRACT

The present invention relates to functional nucleic acid molecules comprising two or more target binding sequences and a regulatory sequence comprising a SINE B2 element or an internal ribosome entry site (IRES). The invention also encompasses methods of enhancing protein translation efficiency, and methods of treating gene defects using the functional nucleic acid molecules of the invention.

### FIELD OF THE INVENTION

The present invention relates to functional nucleic acid molecules comprising two or more target binding sequences and a regulatory sequence comprising a SINE B2 element or an internal ribosome entry site (IRES). Also included are methods of enhancing protein translation, and methods of treating gene defects using the functional nucleic acid molecules of the invention.

### BACKGROUND OF THE INVENTION

SINEUPs are antisense long non-coding RNAs (lncRNAs) that operate post-transcriptionally to upregulate protein expression by increasing translation of cognate mRNAs for which they have specificity. SINEUPs principally utilise two functional domains: an Effector Domain (ED), which mediates upregulation of translation, and a Binding Domain (BD), which comprises an antisense region that provides target specificity. The BD overlaps with the sense transcript and through base complementarity, determines SINEUP specificity. The ED often comprise an embedded Transposable Element (TE), SINEB2, present in an inverted orientation (invSINEB2), which is responsible for translational up-regulation of the target RNA.

Natural SINEUPs are generated from genomic loci that encode overlapping sense/antisense (S/AS) transcript pairs. Antisense transcripts can overlap fully or partially with a cognate sense transcript and, if partially overlapping, may be arranged in a 5′ head-to-head ‘divergent’, or 3′ tail-to-tail ‘convergent’ configuration.

The representative antisense lncRNA ‘antisense transcript to Ubiquitin carboxy-terminal hydrolase 1’ (AS Uchl1), is a 5′ head-to-head ‘divergent’ RNA antisense to the mouse orthologue of human Uch1. Overexpression of AS Uchl1 increases UchL1 protein expression without affecting Uchl1 mRNA levels. AS Uchl1 translational upregulation activity requires the concomitant presence of ED and BD RNA sequences. Under conditions of physiological stress, AS Uchl1 promotes the association of the sense protein-encoding Uchl1 mRNA with heavy polysomes, consequently increasing UCHL1 protein levels without affecting Uchl1 mRNA levels. Further natural human and mouse SINEUP lncRNAs have been identified, suggesting that SINEUPs represent a general class of regulatory RNAs. Artificial SINEUPs can be synthesized by designing BD sequences antisense to a target mRNA (or, according to the present invention, mRNAs) of interest, in order to redirect AS Uchl1 activity to target ectopically expressed transcripts or endogenous m RNAs. In designing synthetic SINEUPs it is of note that the target site (TS) is typically located at the 5′ untranslated region (5′UTR) of an mRNA and can include the ‘AUG’ translation initiation site.

As SINEUPs can increase protein expression of their targets by around 1.5 to 3 fold, they represent an ideal tool to regulate protein expression in vivo, within a physiologically relevant range. For example, protein levels may be upregulated such that they restore protein levels to a physiologically beneficial range, e.g., in disease states characterised by reduced protein levels.

Other regulatory RNA sequences, such as the cis-acting regulatory RNA ‘Internal Ribosome Entry Site’ (IRES) sequences, regulate translation initiation and thereby ultimately modulate protein levels. IRES were first discovered in picornaviruses and were later found to occur in other viral and cellular mRNAs. IRES upregulate target protein levels by promoting translation initiation and are themselves regulated by RNA-binding protein (RBP) IRES trans-acting factors (ITAFs).

The present inventors have previously shown that the invSINEB2 sequence from AS Uchl1 RNA exhibits functional similarity to IRES, and that viral and cellular IRES sequences can act as EDs in synthetic SINEUPs, promoting protein expression in trans. Hence, synthetic functional nucleic acids that are analogous to SINEUPs can be designed that comprise IRESs or functionally active fragments thereof.

Canonical SINEUPS have a single target specificity, a single BD sequence facilitates translational upregulation of one target protein. However, in some disease states, the aberrant state of multiple proteins contributes to the disease phenotype.

Among haploinsufficiencies, there are cases of microdeletions of an entire portion of one of the homologous chromosomes leading to haploinsufficiency of multiple genes. Genetic diseases caused by microdeletions often display a complex phenotype as a result of the involvement of multiple genes. Treating the symptoms of such diseases is often ineffective. Whilst disrupted gene function may be restored by techniques such as gene replacement therapy and RNA therapeutics, these approaches are often limited to targeting single genes with single therapeutics. Thus, complex diseases characterized by abnormalities in multiple proteins, such as microdeletions, have limited therapeutic options.

The genetic disease 22q.11.2 deletion syndrome (22q11.2DS) is characterized by deletions of a portion of the long arm of the 22 chromosome. The deletions can be of different lengths, however a 3 million base (3 Mb) deletion is the most frequent. 22q11.2DS is the most common deletion syndrome and has an estimated frequency of 1 in 3000 to 1 in 6000 live births. Phenotypically, 22q11.2DS exhibits multi-organ dysfunction, including cardiac defects, palatal abnormalities, immune and endocrine problems and various brain function issues. 22q11.2DS patients may display developmental delays, cognitive deficits and neuropsychiatric illness, 22q11.2DS is the most common known genetic cause of schizophrenia.

The present invention seeks to break the one lncRNA to one target paradigm by expanding the number of target mRNAs that can be targeted for translational upregulation using a single functional nucleic acid molecule.

### SUMMARY OF THE INVENTION

Herein, the inventors provide for functional nucleic acids that are both SINEUPs and non-SINE containing lncRNAs (i.e., which contain IRES effector domains or regulatory domains) that comprise multiple binding domains. However, herein the term “SINEUP” may be used to encompass both traditional SINEUPs containing a SINE element as well as corresponding functional nucleic acids containing an IRES.

A functional nucleic molecule disclosed herein may target multiple proteins for translational upregulation.

The inventors provide herein a functional nucleic acid molecule, which comprises multiple target binding domains that are each complementary to a target sequence of an mRNA for which protein translation is to be increased.

The multiple BDs are coupled to the effector functionality of either a SINE B2 sequence or an IRES sequence, or functionally active fragments thereof. Although it will generally be understood that each target binding domain will target a different mRNA, it is envisaged that multiple target binding domains may, in some embodiments, be directed to the same target mRNA, either through the same target binding site of different target binding sites.

Therefore, the functional nucleic acid provided herein facilitates targeted upregulation of one or more proteins of interest.

According to a first aspect of the invention, there is provided a functional nucleic acid molecule comprising:
(a) two or more target binding sequences, wherein each target binding sequence comprises a sequence reverse complementary a target mRNA sequences for which protein translation is to be enhanced, and
(b) a regulatory sequence comprising a SINE B2 element or a functionally active fragment thereof, or an internal ribosome entry site (IRES) or a functionally active fragment thereof.

According to a further aspect of the invention, there is provided a DNA molecule encoding the functional nucleic acid molecule as defined herein.

According to a further aspect of the invention, there is provided an expression vector comprising the functional nucleic acid molecule, or the DNA molecule, as defined herein.

According to a further aspect of the invention, there is provided a composition comprising the functional nucleic acid molecule, the DNA molecule or the expression vector, as defined herein.

According to a further aspect of the invention, there is provided a pharmaceutical composition as defined herein, comprising the functional nucleic acid molecule, the DNA molecule or the expression vector, as defined herein.

According to a further aspect of the invention, there is provided use of the functional nucleic acid molecule, the expression vector or the composition, as defined herein, for enhancing translation of one or more target mRNA sequences.

According to a further aspect of the invention, there is provided the functional nucleic acid molecule, the DNA molecule, the expression vector or the pharmaceutical composition, as defined herein, for use in therapy.

According to a further aspect of the invention, there is provided the functional nucleic acid molecule, the DNA molecule, the expression vector or the pharmaceutical composition, as defined herein, for use in a method of treating a disease associated with gene defects.

According to a further aspect of the invention, there is provided a method of treating a disease associated with gene defects comprising administering the functional nucleic acid molecule, the DNA molecule, the expression vector, the composition or the pharmaceutical composition, as defined herein, to a subject.

According to a further aspect of the invention, there is provided the functional nucleic acid molecule, the DNA molecule, the expression vector, the composition or the pharmaceutical composition, as defined herein, for use in the manufacture of a medicament for treating a gene defect.

### BRIEF DESCRIPTION OF THE FIGURES

FIG. 1—Mono-BD Screening as proof-of-principle for potential multi-BD targets.

Quantification of relative protein levels in the presence of mini-SINEUPs in neuro2A (N2a) cells and astrocytes. Protein levels are indicated as a fold change relative to the negative control, a mini-SINEUP comprising no binding domain (ΔBD). Neuro2A (a) and astrocytes (b) were tested and both cell types were transfected with 1 μg of plasmid expressing the SINEUP for each well of a 6-well plate with lipofectamine 2000. 48 hours after transfection, cells were harvested and processed for western blot (WB) analysis. Data represent means±SEM. n≥4 per group. * p<0.05; ** p<0.01. One-way ANOVA followed by Dunnet's multiple comparison test.

FIG. 2—Multi-BD SINEUPs regulate the expression of multiple proteins.

a) Schematic representation of representative two-BD and five-BD multi-BD SINEUPs. Relative protein expression level change in the presence of multi-BD-SINEUPS 1 (TBX1-DGCR8; 2-BD-SINEUP), 2 (DGCR8-TBX1 (2-BD-SINEUP), 3 (TBX1-RANBP1-SEPT5-ZDHHC8-DGCR8 (5-BD-SINEUP), and 4 (DGCR8-RANBP1-SEPT5-ZDHHC8-TBX1 (5-BD-SINEUP). For all experiments, neuro2a cells were transfected with 1 μg of plasmid expressing the multi-SINEUP for each well of a 6-well plate with lipofectamine 2000. 48 hours after transfection, cells were harvested and processed for protein extraction and western blot (WB) analysis or for RNA extraction and qPCR analysis. Normalization was performed to the housekeeping protein β-actin. Data represent means±SEM. n≥3 per group. * p<0.05. One-way ANOVA followed by Dunnet's multiple comparison test. b) TBX1 relative protein level change (top) and relative mRNA levels (bottom) in the presence of SINEUPs. c) DGCR8 relative protein level change (top) and relative mRNA levels (bottom) in the presence of SINEUPs. d) Relative expression of different SINEUP RNAs. e) RANBP1 relative protein expression level change. f) ZDHHC8 relative protein expression level change. g) SEPT5 relative protein expression level change.

FIG. 3—COMT protein expression can be increased using multi-BD SINEUPs following siRNA-induced downregulation of COMT mRNA

Astrocytes were transfected with different siRNA targeting COMT mRNA (Origene) to downregulate COMT protein levels and then co-transfected with an effective siRNA and the SINEUPs targeting COMT. Cells were transfected with 1 μg of plasmid expressing the SINEUP for each well of a 6-well plate and with the siRNA with siTran 2.0 transfection reagent. 48 hours after the transfection, cells were harvested and processed for protein extraction and western blot (WB) analysis or for RNA extraction and qPCR analysis. Normalization was performed to the housekeeping protein β-actin. Data represent means±SEM. N=2.3 per group. #,* p<0.05. One-way ANOVA followed by Dunnet's multiple comparison test. a) siRNA mediated downregulation of COMT protein expression levels. Three siRNAs, (a, b, c) were obtained from Origene. b) Effect of combined administration of siRNA ‘c’ and multi-BD SINEUPS on COMT protein expression levels. #p<0.05 compared to deltaBD+sc. * p<0.05 compared to deltaBD+c. c) Relative expression of COMT (left) and SINEUP (right) (m)RNAs.

FIG. 4—TBX1, DGCR8 and COMT protein expression can be increased using multi-BD SINEUPs.

Different multi-SINEUP were tested in neuro2a cells or astrocytes. 1=DGCR8-TBX1-COMT (3-BD-SINEUP), 2=DGCR8-TBX1-COMT-RANBP1 (4-BD-SINEUP), 3=DGCR8-TBX1-COMT-ZDHHC8 (4-BD-SINEUP), 4=DGCR8-TBX1-COMT-SEPT5 (4-BD-SINEUP), 5=DGCR8-TBX1-RANBP1-COMT (4-BD-SINEUP), 6=DGCR8-TBX1-ZDHHC8-COMT (4-BD-SINEUP), 7=DGCR8-TBX1-SEPT5-COMT (4-BD-SINEUP). Neuro2a cells were transfected with 1 μg of plasmid expressing the multi-SINEUP for each well of a 6-well plate with lipofectamine 2000. Astrocytes were co-transfected with siRNA c targeting COMT mRNA (Origene) and the multi-SINEUPs. Cells were transfected with 1 μg of plasmid expressing the SINEUP for each well of a 6-well plate and with the siRNA with siTran 2.0 transfection reagent. 48 hours after the transfection, cells were harvested and processed for protein extraction and western blot (WB) analysis or for RNA extraction and qPCR analysis. Normalization was performed to the housekeeping protein R-actin Data represent means±SEM. n≥3 per group. #,* p<0.05. One-way ANOVA followed by Dunnet's multiple comparison test. a) TBX1 relative protein level change (top) and relative mRNA levels (bottom) in the presence of SINEUPs. b) DGCR8 relative protein level change (top) and relative mRNA levels (bottom) in the presence of SINEUPs. c) Relative expression of different SINEUP RNAs. d) COMT relative protein level change (top) and relative mRNA levels (bottom) in the presence of SINEUPs and siRNA. #p<0.05 compared to deltaBD+sc. * p<0.05 compared to deltaBD+c. e) Relative expression of different SINEUP RNAs.

FIG. 5—A multi-BD-SINEUP increases TBX1, DGCR8 and COMT protein expression when expressed in an AAV vector in vitro and in vivo.

An exemplary multi-SINEUP (DGCR8-TBX1-COMT (3-BD-SINEUP)), was cloned in a pAAV expressing the SINEUP under the CAG promoter and the GFP under the PGK promoter. Neuro2a cells were transfected with 1 μg of plasmid expressing the multi-SINEUP for each well of a 6-well plate with lipofectamine 2000. 48 hours after the transfection, cells were harvested and processed for protein extraction and western blot (WB) analysis or for RNA extraction and qPCR analysis. Normalization was performed to the housekeeping protein β-actin. Data represent means±SEM. N=3 per group. * p<0.05. One-way ANOVA followed by Dunnet's multiple comparison test. Then, an AAV1/2 expressing the multi-SINEUP was produced by transfecting HEK293T cells and purified by using heparin column (Hitrap Heparin, Ge-HealthCare Life science). The virus had a titer of 5*10¹¹ vg/ml and was injected (1 μl) in the S1 cortex or in the dorsal striatum. One month after injection, mice were euthanized and brain areas were dissected and lysed for WB and qPCR analysis. Data represent means±SEM. N=3 per group. * p<0.05. Student's T-test was used to compare the two groups. a) in vitro TBX1 relative protein level change (top) and relative mRNA levels (bottom) in the presence of AAV-3-BD-SINEUP. b) in vitro DGCR8 relative protein level change (top) and relative mRNA levels (bottom) in the presence of AAV-3-BD-SINEUP. c) Relative expression of SINEUP RNA using AAV-3-BD-SINEUP. d) in vivo expression of AAV-3-BD-SINEUP elements. e) Quantitative assessment of in vivo change in TBX1 protein levels in the presence of AAV-3-BD-SINEUP. f) Quantitative assessment of in vivo change in DGCR8 protein levels in the presence of AAV-3-BD-SINEUP. g) Quantitative assessment of in vivo change in COMT protein levels in the presence of AAV-3-BD-SINEUP. h) Quantitative assessment of in vivo AAV-3-BD-SINEUP driven 3-BD-SINEUP RNA expression.

### DETAILED DESCRIPTION OF THE INVENTION

It is an object of the present invention to provide a functional nucleic acid molecule comprising two or more target binding sequences and a regulatory sequence comprising a SINE B2 element, or functionally active fragment thereof, or an internal ribosome entry site (IRES), or functionally active fragment thereof, which act post-transcriptionally to increase target protein levels.

Utilising two or more target Binding Domains (BDs), the functional nucleic acid molecule of the invention may be utilised for the targeted upregulation of two or more proteins of interest without affecting mRNA levels. The functional nucleic acid molecule of the invention may be used to enhance translation of target mRNA sequences, such as therapeutic target mRNA sequences which encode therapeutic target proteins, without inducing negative side-effects associated with increasing expression of the target above normal physiological levels.

### Functional Nucleic Acid Molecule

A functional nucleic acid molecule of the present invention comprises two or more target binding sequences, wherein each target binding sequence comprises a sequence reverse complementary to a target mRNA sequence for which protein translation is to be enhanced, and a regulator sequence comprising a SINE B2 element or a functionally active fragment thereof, or an internal ribosome entry site (IRES) or a functionally active fragment thereof.

The “functional nucleic acid molecule” referred to herein is a synthetic molecule of the invention. In particular, the term “functional nucleic acid molecule” describes a nucleic acid molecule (e.g. DNA or RNA) that is capable of enhancing translation of a target mRNA, or target mRNAs, of interest. The term “functional RNA molecule” refers to instances wherein the functional nucleic acid molecule is formed of RNA and said RNA molecule is capable of enhancing the translation of a target mRNA.

A functional nucleic acid molecule according to the invention may be referred to as a trans-acting molecule in that it regulates other nucleic acid molecules, rather than itself.

In a preferred embodiment, the functional nucleic acid molecule of the invention is an RNA molecule.

In one embodiment, the functional nucleic acid molecule further comprises at least one spacer sequence between the two or more target binding sequences and the regulatory sequence. SEQ ID NOs: 1 and 136 are non-limiting example of the spacer/linker sequence which may be used in the present invention.

In one embodiment, the spacer/linker sequence may comprise SEQ ID NO: 1.

In one embodiment, the spacer/linker sequence may consist of SEQ ID NO: 1.

In one embodiment, the spacer/linker sequence may comprise SEQ ID NO: 136.

In one embodiment, the spacer/linker sequence may consist of SEQ ID NO: 136.

The functional nucleic acid molecule provided herein may trans-acting such that it functionally modulates sequences present on other RNA molecules. In one embodiment, the functional nucleic acid molecule provided herein is a trans-acting functional nucleic acid molecule.

In one embodiment, the functional nucleic acid molecule is single stranded.

In one embodiment, the functional nucleic acid molecule comprises RNA nucleotides.

The functional nucleic acid molecule of the present invention preferably comprises RNA nucleotides.

In one embodiment, the functional nucleic acid molecule consists of RNA nucleotides.

The functional nucleic acid molecule of the present invention preferably consists of RNA nucleotides.

In one embodiment, the functional nucleic acid molecule is RNA.

The functional nucleic acid molecule of the present invention preferably is RNA.

In one embodiment, the functional nucleic acid molecule comprises DNA nucleotides.

In one embodiment, the functional nucleic acid molecule consists of DNA nucleotides.

In one embodiment, the functional nucleic acid molecule is RNA.

In one embodiment, the functional nucleic acid molecule comprises one or more modifications or chemical modifications.

The term “modification” or “chemical modification” refers to a structural change in, or on, the most common, natural ribonucleotides: adenosine, guanosine, cytidine, thymidine, or uridine ribonucleotides. In particular, the chemical modifications described herein may be changes in or on a nucleobase (i.e. a chemical base modification), or in or on a sugar (i.e. a chemical sugar modification). The chemical modifications may be introduced co-transcriptionally (e.g. by substitution of one or more nucleotides with a modified nucleotide during synthesis), or post-transcriptionally (e.g. by the action of an enzyme).

Chemical modifications are known in the art, for example as described in The RNA Modification Database provided by The RNA Institute (https://mods.ma.albany.edu/mods/). Examples of chemical modifications which may be useful in the present invention are described in PCT/GB2021/052607, which is incorporated herein by reference in its entirety.

In one embodiment, the chemical modification is a chemical base modification. The chemical base modification may be selected from a modification of an adenine, cytosine and/or uracil base.

In one embodiment, the chemical base modification is selected from methylation and/or isomerisation. In a further embodiment, the chemical base modification is selected from the group consisting of: Pseudouridine (ψ), N1-Methylpseudouridine (N1mψ), 5-Methylcytidine (m5C) and N6-Methyladenosine (m6A). In a further embodiment, the chemical base modification is selected from the group consisting of: Pseudouridine, N1-Methylpseudouridine and N6-Methyladenosine.

In one embodiment, the chemical modification is a chemical sugar modification. In one embodiment, the chemical sugar modification is methylation. In one embodiment, the chemical sugar modification is a 2′ modification, such as a 2′-O-Methyl modification. In a further embodiment, the chemical sugar modification is 2′-O-Methyladenosine (Am).

In one embodiment, the functional nucleic acid molecule comprises a 3′-polyadenylation (polyA) tail. A “3′-polyA tail” refers to a long chain of adenine nucleotides added to the 3′-end of the functional nucleic acid which provides stability to the RNA molecule and can promote translation.

In one embodiment the functional nucleic acid molecule comprises a 5′-cap. A “5′-cap” refers to an altered nucleotide at the 5′-end of the transcript which provides stability to the molecule, particularly from degradation from exonucleases, and can promote translation.

Most commonly, the 5′-cap may be a 7-methylguanylate cap (m7G), i.e. a guanine nucleotide connected to the RNA via a 5′ to 5′ triphosphate linkage and methylated on the 7 position.

The functional nucleic acid herein may constitute a miniSINEUP or microSINEUP, as defined in WO 2019/150346 and PCT/GB2021/052502, which are incorporated herein by reference in their entirety. By the term “miniSINEUP” there is intended a functional nucleic acid molecule comprising (or consisting of) two or more target binding domains (i.e. complementary sequences to target mRNAs), optionally a spacer sequence, and any SINE or IRES sequence as the effector domain (Zucchelli et al., Front Cell Neurosci., 9: 174, 2015).

By the term “microSINEUP” there is intended a functional nucleic acid molecule comprising (or consisting of) two or more target binding domains (i.e. complementary sequences to target mRNAs), optionally a spacer sequence, and a functionally active fragment of the SINE or IRES sequence.

In one embodiment the functional nucleic acid may be circular.

### Target Binding Sequences

The target binding sequence (also referred to as the target determinant sequence) is the portion of the functional nucleic acid molecule that binds to the target m RNA.

In preferred embodiments wherein the functional nucleic acid molecule is a functional RNA molecule, the target binding sequence is the portion of the functional RNA molecule that binds to the target mRNA.

The functional nucleic acid molecule of the invention comprises two or more target binding sequences. In one embodiment, the functional nucleic acid molecule comprises 2, 3, 4, 5, 6 7, 8, 9, 10 or more, target-binding sequences.

In one embodiment, the functional nucleic acid molecule consists of 2, 3, 4, 5, 6, 7, 8, 9, 10 or more, target-binding sequences

In one embodiment, the functional nucleic acid molecule comprises two target binding sequences.

In another embodiment, the functional nucleic acid molecule comprises three target binding sequences.

In another embodiment, the functional nucleic acid molecule comprises four target binding sequences.

In another embodiment, the functional nucleic acid molecule comprises four target binding sequences.

In another embodiment, the functional nucleic acid molecule comprises five target binding sequences.

In another embodiment, the functional nucleic acid molecule comprises six target binding sequences.

In another embodiment, the functional nucleic acid molecule comprises seven target binding sequences.

In another embodiment, the functional nucleic acid molecule comprises eight target binding sequences.

In one embodiment, the functional nucleic acid molecule comprises nine target binding sequences.

In one embodiment, the functional nucleic acid molecule comprises ten target binding sequences.

It would be understood to one skilled in the art that the number of target binding sequences reflects the number of target sequences intended to be targeted by the functional nucleic acid molecule of the invention.

The two or more target binding sequences may target one or more mRNAs. It will be understood that a functional nucleic acid molecule of the invention may target one or more mRNAs whilst possessing to or more target binding sequences since at least two of the two or more target binding sequences may comprise a sequence reverse complementary to target sequences contained within the same target mRNA, which may be the same target sequence or a different target sequence. Thus at least two of the two or more target binding sequences may be directed towards the same protein for translational upregulation.

However, it will be understood by the skilled person that in many embodiments each target binding sequence will be reverse complementary to a sequence within a different mRNA. In such embodiments a functional nucleic acid molecule containing two target binding sequences will target two m RNAs, a functional nucleic acid molecule containing three target binding sequences will target three mRNAs, a functional nucleic acid molecule containing four target binding sequences will target four mRNAs, and so on.

The target binding sequences may be separated from one another and from other functional elements by nucleic acid sequences comprising “spacers”. In some embodiments, the two or more target binding sequences may be separated from one another by spacers.

In one embodiment the target binding sequences are separated by a spacer.

In one embodiment the target binding sequences are separated by a spacer, wherein the spacer is 19 nucleotides in length.

In one aspect, the two or more target binding sequences each comprise a sequence reverse complementary to a target mRNA sequence for which protein translation is to be enhanced.

In one aspect, the two or more target binding sequences each comprise a sequence reverse complementary to a therapeutic target m RNA sequence for which protein translation is to be enhanced.

As used herein, “therapeutic target” or “therapeutic target mRNA sequence” refers to a target which may be used to treat a disease or condition in a subject when its translation is enhanced, such as enhanced by using a functional nucleic acid molecule according to the present invention.

For example when expressed in a subject (such as in a cell of a subject), a therapeutic target may: replace a protein that is deficient or abnormal in a cell; augment an existing pathway in a cell; and/or provide a novel function or activity in a cell; thereby treating a disease or condition of said subject. Herein, the use of target binding domains which enhance translation of multiple proteins can be used to treat a disease or condition in which multiple proteins are affected.

In one embodiment, the therapeutic target comprises at least one gene defect.

In one embodiment the at least one gene defect may be haploinsufficiency.

The functional nucleic acid molecule described herein may comprise a spacer between the two or more target binding sequences. In one embodiment, there is provided is a functional nucleic acid molecule, wherein the two or more target binding sequences are separated by a spacer. In a preferred embodiment, said spacer is 19 nucleotides in length.

In WO 2012/133947, which is incorporated herein by reference in its entirety, it was shown that a target binding sequence needs to have only about 60% similarity with a sequence reverse complementary to the target mRNA. In fact, the target binding sequence can even display a large number of mismatches and retain activity.

The target binding sequences of the functional nucleic acid molecule of the invention may each display about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or about 100% similarity with a sequence reverse complementary to the target mRNA.

The target binding sequences of the functional nucleic acid molecule of the invention may each display about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or about 100% identity with a sequence reverse complementary to the target mRNA.

Herein, polypeptide or polynucleotide sequences are said to be the same as or “identical” to other polypeptide or polynucleotide sequences, if they share 100% sequence identity over their entire length. Residues in sequences are numbered from left to right, i.e. from N- to C-terminus for polypeptides; from 5′ to 3′ terminus for polynucleotides. If closely-related sequences are not identical they may be similar, i.e., they may possess a certain degree of sequence identity (or similarity) across a given range, a sequence may be 50%, 60%, 70%, 80%, 90%, or 99% similar to another sequence. Unless a specific reference range is given, e.g., with respect to the nucleotide positions, any quoted sequence similarity or identity will be understood as being calculated across the entire length of the shorter of the sequences being used in the comparison, (unless otherwise stated). For example, a sequence of 100 nt in length may be 98% identical to a sequence 1000 nt in length if, in the aligned region of 100 nts, only two nucleotides differ.

For the purposes of comparing two closely-related polynucleotide sequences, the “% sequence identity” between a first nucleotide sequence and a second nucleotide sequence may be calculated using NCBI BLAST, using standard settings for nucleotide sequences (BLASTN). For the purposes of comparing two closely-related polypeptide sequences, the “% sequence identity” between a first polypeptide sequence and a second polypeptide sequence may be calculated using NCBI BLAST, using standard settings for polypeptide sequences (BLASTP). A “difference” between sequences refers to an insertion, deletion or substitution of a single nucleotide in a position of the second sequence, compared to the first sequence. Insertions, deletions or substitutions in a second sequence which is otherwise identical (100% sequence identity) to a first sequence result in reduced % sequence identity.

“Complementarity” relates to the Watson-Crick base pairing principle that ‘A’ nucleotides will hydrogen bond with ‘T’ (or ‘U’) nucleotides, and ‘G’ nucleotides with ‘C’ nucleotides to form double stranded structures that associate via said “complementary” nucleotides. Herein, a “complementary” sequence is a sequence closely-related to another sequence such that such base pairing can occur. Complementary sequences may be 100% complementary such that they may base pair across their entire length, or they may be e.g., 99%, 90%, 80%, 70%, or 60% complementary etc., such that they base pair across portions of their sequence. Here, as is common in the art, a complementary sequence may also be called a “reverse complementary” sequence.

The target binding sequence comprises a sequence which is sufficient in length to bind to the target mRNA transcript. Therefore, the target binding sequence may be at least about 10 nucleotides in length, such as at least about 14 nucleotides in length, such as at least about 15 nucleotides in length, such as at least about 16 nucleotides in length, such as at least about 17 nucleotides in length, such as least 18 nucleotides in length. Furthermore, the target binding sequence may be less than about 250 nucleotides in length, preferably less than about 200 nucleotides in length, less than about 150 nucleotides in length, less than about 140 nucleotides in length, less than about 130 nucleotides in length, less than about 120 nucleotides in length, less than about 110 nucleotides in length less than about 100 nucleotides in length, less than about 90 nucleotides in length, less than about 80 nucleotides in length, less than about 70 nucleotides in length, less than about 60 nucleotides in length or less than about 50 nucleotides in length. In one embodiment, the target binding sequence is between about 4 and about 50 nucleotides in length, such as between about 18 and about 44 nucleotides in length.

The target binding sequence may be designed to hybridise with the 5′-untranslated region (5′ UTR) of the target mRNA sequence. In one embodiment, the sequence is reverse complementary to 0 to 50 nucleotides, such as 0 to 40, 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 210 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, or 0 to 6 nucleotides of the 5′ UTR.

Alternatively, or in combination, the target binding sequence may be designed to hybridise to the coding sequence (CDS) of the target mRNA sequence. In one embodiment, the sequence is reverse complementary to 0 to 40 nucleotides, such as 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, or 0 to 4 nucleotides of the CDS.

The target binding sequence may be designed to hybridise to a region upstream of an AUG site (start codon), such as a start codon within the CDS, of the target mRNA sequence. In one embodiment, the sequence is reverse complementary to 0 to 80 nucleotides, such as 0 to 70, 0 to 60, 0 to 50, 0 to 40, 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, or 0 to 9 nucleotides of the AUG site. Alternatively, or in combination, the target binding sequence may be designed to hybridise to the target mRNA sequence downstream of said AUG site. In one embodiment, the sequence is reverse complementary to 0 to 40 nucleotides, such as 0 to 39, 0 to 38, 0 to 37, 0 to 36, 0 to 35, 0 to 34, 0 to 33, 0 to 32, 0 to 31, 0 to 30, 0 to 29, 0 to 28, 0 to 27, 0 to 26, 0 to 25, 0 to 24, 0 to 23, 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, or 0 to 4 nucleotides of the target mRNA sequence downstream of said AUG site.

In one embodiment, the target determinant sequence is at least 10 nucleotides in length and comprises, from 3′ to 5′:
a sequence reverse complementary to 0 to 50 nucleotides of the 5′ untranslated region (5′ UTR) and 0 to 40 nucleotides of the coding sequence (CDS) of the target m RNA sequence; or
a sequence reverse complementary to 0 to 80 nucleotides of the region upstream of an AUG site (start codon) of the target mRNA and 0 to 40 nucleotides of the CDS of the target mRNA sequence downstream of said AUG site.

In one embodiment, the target determinant sequence is at least 14 nucleotides in length and comprises, from 3′ to 5′:
a sequence reverse complementary to 0 to 40 nucleotides of the 5′ UTR and 0 to 32 nucleotides of the CDS of the target mRNA sequence; or
a sequence reverse complementary to 0 to 70 nucleotides of the region upstream of an AUG site (start codon) of the target mRNA and 0 to 4 nucleotides of the CDS of the target mRNA sequence downstream of said AUG site.

In one embodiment, the coding sequence starts on the first AUG site (M1) of the mRNA.

In one embodiment, the preferred AUG site is that corresponding to an internal start codon (e.g. M2).

In the context of referencing a sequence reverse complementary to a region in the 5′ UTR and the CDS, this is preferably anchored around the AUG site, i.e. the region in the 5′ UTR is directly upstream of the AUG site of the target mRNA. For example, reference to a target binding sequence that is “−40/+4 of M1” refers to a target binding sequence that is reverse complementary to the 40 nucleotides within the 5′ UTR upstream of the AUG site (−40) and the 4 nucleotides within the CDS downstream of the AUG site (+4).

In accordance with conventional numbering, the nucleotides of the 5′UTR sequence are numbered sequentially using decreasing negative numbers approaching the AUG site on the target mRNA (e.g. −3, −2, −1). The nucleotides of the CDS sequence are numbered sequentially using increasing positive numbers (e.g. +1, +2, +3) from the AUG site, such that the A of the AUG site is numbered +1. The region bridging the 5′UTR and the CDS will therefore be numbered −3, −2, −1, +1, +2, +3, with the A of the AUG site numbered +1.

It is to be understood that “the target binding sequence” refers independently to each of the two or more target binding sequences. For example, one of the two or more target binding sequence may be 20 nt long and have 80% sequence identity to its target mRNA whilst another may be 30 nt long and have 95% sequence identity to its target mRNA.

Each of the two or more target binding sequences may be designed independently of one another.

In certain embodiments, the two or more target binding sequences may be the same.

In other embodiments, the two or more target binding sequences may be different.

In preferred embodiments, the two or more target binding sequences may be directed towards different proteins for which translation is to be enhanced.

Exemplary Target mRNAs and Target Binding Sequences

The target mRNAs (also called target mRNA sequences) of the invention may constitute any mRNA for which upregulation of the protein encoded thereby is sought.

Without limitation, target mRNAs may be any mRNAs encoding TBX-1, HIRA1, DGCR8, PRODH, COMT, RANBP1, ZDHHC8, SEPT5 or RTN4R.

In one embodiment, the target mRNA sequences for which protein translation is to be enhanced encodes TBX-1. T-Box Transcription Factor 1 (TBX-1) is a member of the T-box family of binding domain transcription factors and is the most studied gene in 22q11.2DS.

Mice haploinsufficient for TBX-1 recapitulate major phenotypes of the disease including cardiac and thymic defects and abnormal growth of the pharyngeal arch. TBX-1 is important for brain microvasculature development and is thus linked to the development of cognitive and psychiatric disease phenotypes.

In one embodiment, the target mRNA sequence comprises or consists of a sequence as set forth in any one of SEQ ID NOs: 73, 74, and 75, which correspond to mouse TBX-1 mRNA transcripts with the respective NCBI reference codes NM_011532.2, NM_001285476.1, and NM_001285472.1.

In one embodiment, the target mRNA sequences for which protein translation is to be enhanced encodes DGCR8. DGCR8 (DGCR8 Microprocessor Complex Subunit) is encoded by another gene (DGCR8) that maps within the deleted region on chromosome 22 in 22q11.2DS. DGCR8 encodes a protein involved in the biogenesis of miRNA, thus its deficiency may influences multiple pathways. Mice haploinsufficient for DGCR8 show neural deficits similar of the ones observed in 22q11.2DS.

In one embodiment, the target mRNA sequence comprises or consists of a sequence as set forth in SEQ ID NO: 76, which corresponds to a mouse DGCR8 m RNA transcript with the NCBI reference code NM_033324.2.

In one embodiment, the target mRNA sequences for which protein translation is to be enhanced encodes COMT. Catechol-O-methyltransferase (COMT) is an enzyme implicated in dopamine degradation. COMT is highly expressed in the prefrontal cortex, a brain region important for higher cognitive functions. A Polymorphism in COMT gene has been described, which can lead to a reduction in enzymatic activity which leads to an accumulation of dopamine in the synaptic cleft, thereby influencing cognitive performances both in human and in transgenic mice. COMT has been linked to psychiatric disorders and schizophrenia. COMT is one of the haploinsufficient genes associated with 22q11.2DS, as such, the above polymorphism serves as an example of the phenotypic effects associated with reduced COMT protein activity, which may be similar to the possible phenotypic effects associated with a reduction in the total amount of COMT protein produced as a result of haploinsufficiency.

In one embodiment, the target mRNA sequence comprises or consists of a sequence as set forth in SEQ ID NO: 77, which corresponds to a mouse COMT mRNA transcript with the NCBI reference code NM_001111062.1.

In one embodiment, the target mRNA sequences for which protein translation is to be enhanced encodes HIRA1. The HIRA1 gene encodes for a nuclear protein with histone-binding properties that have been conserved from yeast to humans. Several HIRA binding proteins have also been described, including Pax3, a homeodomain protein critical for patterning and embryogenesis.

In one embodiment, the target mRNA sequence comprises or consists of a sequence as set forth in SEQ ID NO: 78, which corresponds to a mouse HIRA1 mRNA transcript with the NCBI reference code NM_010435.2.

In one embodiment, the target mRNA sequences for which protein translation is to be enhanced encodes PRODH. The PRODH gene encodes for the proline dehydrogenase enzyme, which is involved in the degradation of proline, an agonist of glutamatergic receptors and potentiator of excitatory neurotransmission.

In one embodiment, the target mRNA sequence comprises or consists of a sequence as set forth in SEQ ID NO: 79, which corresponds to a mouse PRODH mRNA transcript with the NCBI reference code NM_011172.2.

In one embodiment, the target mRNA sequences for which protein translation is to be enhanced encodes ZDHHC8. The ZDHHC8 gene encodes a PAT enzyme, which adds a palmitoyl chemical group to proteins to anchor them to cell membranes. It plays an important role in regulating nervous system development, dendritic morphology, spine density, synaptic proteins, and glutamatergic neurotransmission.

In one embodiment, the target mRNA sequence comprises or consists of a sequence as set forth in SEQ ID NO: 80, which corresponds to a mouse ZDHHC8 mRNA transcript with the NCBI reference code NM_172151.4.

In one embodiment, the target mRNA sequences for which protein translation is to be enhanced encodes RANBP1. RANBP1 encodes a binding protein for the small GTPase Ran. As a regulator of the Ran complex, this protein has multiple functions, including cilia formation and modulation of mitosis. Evidence for a role in neurogenesis places RANBP1 as a candidate for the cortical circuits implicated in disorders associated with 22q11.2DS, such as attention-deficit disorders, autism and schizophrenia.

In one embodiment, the target mRNA sequence comprises or consists of a sequence as set forth in SEQ ID NO: 81, which corresponds to a mouse RANBP1 mRNA transcript with the NCBI reference code NM_011239.2.

In one embodiment, the target mRNA sequences for which protein translation is to be enhanced encodes SEPT5. SEPT5 belongs to the Septin family and is expressed predominantly in the mammalian brain. It is localized in presynaptic terminals where it is physically associated with synaptic vesicles and other membranes. The N and C termini of Sept5 interact with syntaxin 1A, which is involved in the regulation of exocytosis.

In one embodiment, the target mRNA sequence comprises or consists of a sequence as set forth in SEQ ID NO: 82, which corresponds to a mouse SEPT5 mRNA transcript with the NCBI reference code NM_213614.2.

In one embodiment, the target mRNA sequences for which protein translation is to be enhanced encodes RTN4R. RTN4R encodes NOGO receptor 1, regulates axonal growth as well as axon regeneration after injury and has also been considered as a potential susceptibility gene for schizophrenia.

In one embodiment, the target mRNA sequence comprises or consists of a sequence as set forth in SEQ ID NO: 83, which corresponds to a mouse RTN4R mRNA transcript with the NCBI reference code NM_022982.3.

In one embodiment, the functional nucleic acid molecule comprises a target binding sequence complementary to a target mRNA sequence encoding one or more of TBX-1, HIRA1, DGCR8, PRODH, COMT, RANBP1, ZDHHC8, SEPT5 and RTN4R.

In one embodiment, the functional nucleic acid molecule comprises target binding sequences complementary to target mRNA sequences encoding two or more of TBX-1, HIRA1, DGCR8, PRODH, COMT, RANBP1, ZDHHC8, SEPT5 and RTN4R.

In one embodiment, the functional nucleic acid molecule comprises target binding sequences complementary to target mRNA sequences encoding three or more of TBX-1, HIRA1, DGCR8, PRODH, COMT, RANBP1, ZDHHC8, SEPT5 and RTN4R.

In one embodiment, the functional nucleic acid molecule comprises target binding sequences complementary to target m RNA sequences encoding four or more of TBX-1, HIRA1, DGCR8, PRODH, COMT, RANBP1, ZDHHC8, SEPT5 and RTN4R.

In one embodiment, the functional nucleic acid molecule comprises target binding sequences complementary to target mRNA sequences encoding five or more of TBX-1, HIRA1, DGCR8, PRODH, COMT, RANBP1, ZDHHC8, SEPT5 and RTN4R.

In one embodiment, the functional nucleic acid molecule comprises target binding sequences complementary to target mRNA sequences encoding six or more of TBX-1, HIRA1, DGCR8, PRODH, COMT, RANBP1, ZDHHC8, SEPT5 and RTN4R.

In one embodiment, the functional nucleic acid molecule comprises target binding sequences complementary to target mRNA sequences encoding seven or more of TBX-1, HIRA1, DGCR8, PRODH, COMT, RANBP1, ZDHHC8, SEPT5 and RTN4R.

In one embodiment, the functional nucleic acid molecule comprises target binding sequences complementary to target mRNA sequences encoding eight or more of TBX-1, HIRA1, DGCR8, PRODH, COMT, RANBP1, ZDHHC8, SEPT5 and RTN4R.

In one embodiment, the functional nucleic acid molecule comprises target binding sequences complementary to target mRNA sequences encoding TBX-1, HIRA1, DGCR8, PRODH, COMT, RANBP1, ZDHHC8, SEPT5 and RTN4R.

In one embodiment one of the target binding sequences is reverse complementary to any one of SEQ ID NO: 73-109.

In one embodiment one of the target binding sequences is reverse complementary to a sequence having at least about 60%, at least about 70%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% sequence identity to a sequence selected from any one of SEQ ID NOs: 73-109.

In one embodiment, the functional nucleic acid molecule comprises target binding sequences complementary to any one of SEQ ID NOs: 73-109.

As discussed above, the two or more target binding sequences of the functional nucleic acid molecule may be directed towards the same target mRNA, either through the same target region or different target regions. Therefore, the two or more target binding sequences of the functional nucleic acid of the invention are complimentary to one or more target mRNA sequences.

In one embodiment, one or more of the target binding sequences comprises a sequence selected from the group consisting of SEQ ID NOs: 110-135.

In one embodiment, one or more of the target binding sequences consists of a sequence selected from the group consisting of SEQ ID NOs: 110-135.

In one embodiment, the functional nucleic acid molecule comprises target binding sequences complementary to target mRNA sequences encoding DGCR8, TBX1 and COMT.

In one embodiment, the functional nucleic acid molecule comprises target binding sequences that are complementary to target mRNA sequences as set forth in any two or more of SEQ ID NOs: 73-77, 84-87, 91-93, and 106-109, or a fragment thereof.

In another embodiment, the functional nucleic acid molecule comprises target binding sequences that are complementary to target mRNA sequences as set forth in any two or more of SEQ ID NOs: 93, 87, and 108.

In one embodiment, the target binding sequences comprise SEQ ID NO: 119, 113, and/or 134.

In one embodiment, the target binding sequences consist of SEQ ID NO: 119, 113, and/or 134.

The target binding sequences may be further defined in terms of their relative position within the functional nucleic acid. In one embodiment, the target binding sequences comprise, from 5′ to 3′, sequences that target DGCR8 −14/+4 M2, TBX1 −10/+8 M4, and COMT −40/+4 M2.

In one embodiment, the target binding sequences comprise, from 5′ to 3′, SEQ ID NOs 119, 113, and 134.

In one embodiment, the target binding sequences consist of, from 5′ to 3′, SEQ ID NOs 119, 113, and 134.

A functional nucleic acid molecule of the invention may increase the level of TBX-1, DGCR8 and COMT protein without exceeding non-disease state levels. Said functional nucleic acid may therefore ameliorate a disease or disorder of the nervous system associated with reduced levels of TBX-1, DGCR8 and COMT proteins.

In an embodiment, the functional nucleic acid molecule of the invention increases the level of TBX-1, DGCR8 and COMT proteins.

In an embodiment, the functional nucleic acid molecule of the invention increases the level of TBX-1, DGCR8 and COMT proteins to ameliorate unwanted phenotypes associated with abnormally low levels of the foregoing proteins.

TABLE 1
Exemplary BD target & target-binding sequences

| | | SEQ ID | Target-binding | SEQ ID |
|---|---|---|---|---|
| Target identity | Target sequence | NO: | sequence | NO: |
| TBX1 −30/+4 | CTCTGATCAGCGGCG | 84 | TCATGACCCGAAGG | 110 |
| (M1) | GGTGGCCTTCGGGTC | | CCACCCGCCGCTGA | |
| | ATGA | | TCAGAG | |
| TBX1 −14/+4 | GTGGCCTTCGGGTCA | 85 | TCATGACCCGAAGG | 111 |
| (M1) | TGA | | CCAC | |
| TBX1 −10/+34 | AGGCAGACGAATGTT | 86 | TTCCAAAAAGCTTCA | 112 |
| (M4) | CCCCACGTTCCAAGT | | CTTGGAACGTGGGG | |
| | GAAGCTTTTTGGAA | | AACATTCGTCTGCCT | |
| TBX1 −10/+8 | AGGCAGACGAATGTT | 87 | GGGAACATTCGTCT | 113 |
| (M4) | CCC | | GCCT | |
| HIRA −40/+4 | AGGGACACGGCTCTG | 88 | TCATTGTTCGGCCGC | 114 |
| (M1) | GCTTCAGCCCGGCAG | | TGCCGGGCTGAAGC | |
| | CGGCCGAACAATGA | | CAGAGCCGTGTCCC | |
| | | | T | |
| HIRA −14/+4 | GCAGCGGCCGAACA | 89 | TCATTGTTCGGCCGC | 115 |
| (M1) | ATGA | | TGC | |
| HIRA −14/+4 | TTGTGATCTGGAATAT | 90 | ACATATTCCAGATCA | 116 |
| (M2) | GT | | CAA | |
| DGCR8 −40/+4 | AAAACTCTGGTCTTTT | 91 | CCATATTATGAGCAG | 117 |
| (M1) | AAAGTAGTCTTAACT | | TTAAGACTACTTTAA | |
| | GCTCATAATATGG | | AAGACCAGAGTTTT | |
| DGCR8 −14/+4 | CTTAACTGCTCATAAT | 92 | CCATATTATGAGCAG | 118 |
| (M1) | ATGG | | TTAAG | |
| DGCR8 −14/+4 | CCGCAGGAGAAGCG | 93 | TCATCGCTTCTCCTG | 119 |
| (M2) | ATGA | | CGG | |
| PRODH−33/+4 | TTAAGCCTCCTGAGC | 94 | CCATGGCGGAGCCG | 120 |
| (M1) | GCGTGTATCGGCTCC | | ATACACGCGCTCAG | |
| | GCCATGG | | GAGGCTTAA | |
| PRODH−14/+4 | GTATCGGCTCCGCCA | 95 | CCATGGCGGAGCCG | 121 |
| (M1) | TGG | | ATAC | |
| PRODH −1/+18 | ATGGCTCTCAAGCGC | 96 | GACGCGCTTGAGAG | 122 |
| (M1) | GTC | | CCAT | |
| RANBP1 −40/+4 | GCGACGCCTGAGCTA | 97 | CCATGGGGGCGCGG | 123 |
| (M1) | GTCGAGCCACCGTCG | | CGACGGTGGCTCGA | |
| | CCGCGCCCCCATGG | | CTAGCTCAGGCGTC | |
| | | | GC | |
| RANBP1 −14/+4 | GTCGCCGCGCCCCC | 98 | CCATGGGGGCGCGG | 124 |
| (M1) | ATGG | | CGAC | |
| RANBP1 −1/+18 | ATGGCGGCCGCCAA | 99 | GTCCTTGGCGGCCG | 125 |
| (M1) | GGAC | | CCAT | |
| ZDHHC8 −40/+4 | CGCCCGGCCCGGGG | 100 | GCATCCTGGGCGCC | 126 |
| (M1) | AGGGATGCGGCGGC | | GCGCCGCCGCATCC | |
| | GCGGCGCCCAGGAT | | CTCCCCGGGCCGGG | |
| | GC | | CG | |
| ZDHHC8 −14/+4 | GCGCGGCGCCCAGG | 101 | GCATCCTGGGCGCC | 127 |
| (M1) | ATGC | | GCGC | |
| ZDHHC8 −1/+18 | ATGCCCCGCAGCCCC | 102 | CCCGGGGCTGCGGG | 128 |
| (M1) | GGG | | GCAT | |
| RTN4R −40/+4 | GCCGCTTCCAGTGCC | 103 | TCATCTTCGGGGTC | 129 |
| (M1) | CGACGCGCCCCGCT | | GAGCGGGGCGCGTC | |
| | CGACCCCGAAGATGA | | GGGCACTGGAAGCG | |
| | | | GC | |
| RTN4R −14/+4 | GCTCGACCCCGAAGA | 104 | TCATCTTCGGGGTC | 130 |
| (M1) | TGA | | GAGC | |
| RTN4R −1/+18 | ATGAAGAGGGCGTCC | 105 | GGAGGACGCCCTCT | 131 |
| (M1) | TCC | | TCAT | |
| COMT −1/+40 | ATGCTGTTGGCTGCT | 106 | CCAGCAACAGGAGA | 132 |
| (M1) | GTCTCATTGGGTCTC | | CCCAATGAGACAGC | |
| | CTGTTGCTGG | | AGCCAACAGCAT | |
| COMT −1/+18 | ATGCTGTTGGCTGCT | 107 | GACAGCAGCCAACA | 133 |
| (M1) | GTC | | GCAT | |
| COMT −40/+4 | TTGGTTTGAGTTCGT | 108 | CCATGAGCAGGTTG | 134 |
| (M2) | GCAGCAGCCGGTCC | | TGGACCGGCTGCTG | |
| | ACAACCTGCTCATGG | | CACGAACTCAAACCA | |
| | | | A | |
| COMT −14/+4 | TCCACAACCTGCTCA | 109 | CCATGAGCAGGTTG | 135 |
| (M2) | TGG | | TGGA | |

### Regulatory Sequences

The functional nucleic acids of the invention comprise a regulatory sequence comprising a SINE B2 element or a functionally active fragment thereof, or an internal ribosome entry site (IRES) or a functionally active fragment thereof.

In accordance with SINEUP nomenclature or by analogy to SINEUPs (where IRES are used), regulatory sequences may also be known as effector domains (EDs).

The regulatory sequence has translation enhancing activity such that protein production is increased. Increased or enhanced protein translation activity indicates that the efficiency or activity of translation is increased as compared to a case where the functional nucleic acid molecule according to the present invention is not present in a system.

The regulatory sequence has protein translation enhancing activity.

The regulatory sequence increases or enhanced translation of target mRNAs.

The functional nucleic acid molecule of the invention is applicable to uses and methods for enhancing translation of one or more target mRNA sequences.

It will be understood that by “enhancing translation of one or more target mRNA sequences” it is meant that translation of the entire protein-coding region of the target m RNA will be enhanced such that synthesis of the protein encoded thereby is increased.

In one embodiment, expression of the protein encoded by the target mRNA is increased by at least 1.2 fold, such as at least 1.5 fold, in particular at least 2 fold.

In a further embodiment, expression of the protein encoded by the target mRNA is increased between 1.5 to 3 fold, such as between 1.6 and 2.2 fold.

It will be understood that by “protein expression”, it is meant the level of protein present in a system as determined by the transcriptional activity within that system. For example, increasing protein expression will be understood to mean ultimately increasing the amount of a given protein in the system.

In one embodiment the expression of the protein encoded by the target mRNA is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2.0 fold, at least about 2.1 fold, at least about 2.2 fold, at least about 2.3 fold, at least about 2.4 fold, at least about 2.5 fold, at least about 2.6 fold, at least about 2.7 fold, at least about 2.8 fold, at least about 2.9 fold, or at least about 3.0 fold.

In one embodiment the expression of the protein encoded by the target mRNA is increased about 1.1 fold, about 1.2 fold, about 1.3 fold, about 1.4 fold, about 1.5 fold, about 1.6 fold, about 1.7 fold, about 1.8 fold, about 1.9 fold, about 2.0 fold, about 2.1 fold, about 2.2 fold, about 2.3 fold, about 2.4 fold, about 2.5 fold, about 2.6 fold, about 2.7 fold, about 2.8 fold, about 2.9 fold, or about 3.0 fold.

In one embodiment the expression of the protein encoded by the target mRNA is increased by less than about 1.2 fold, less than about 1.3 fold, less than about 1.4 fold, less than about 1.5 fold, less than about 1.6 fold, less than about 1.7 fold, less than about 1.8 fold, less than about 1.9 fold, less than about 2.0 fold, less than about 2.1 fold, less than about 2.2 fold, less than about 2.3 fold, less than about 2.4 fold, less than about 2.5 fold, less than about 2.6 fold, less than about 2.7 fold, less than about 2.8 fold, less than about 2.9 fold, or less than about 3.0 fold.

These increases in protein expression are within physiological ranges. It is envisaged that increasing protein expression within these ranges will allow the treatment of diseases associated with one or more gene defects, such as cancer or neurodegenerative diseases, without leading to negative side effects associated with increasing expression of the target above non-disease state or ‘wild-type’ physiological levels.

It is understood that by “the target mRNA” it is meant each of the one or more target mRNA sequences.

In one embodiment, the regulatory sequence is located 3′ of the target binding sequence. The regulatory sequence may be in a direct or inverted orientation relative to the 5′ to 3′ orientation of the functional nucleic acid molecule. Reference to “direct” refers to the situation in which the regulatory sequence is embedded (inserted) with the same 5′ to 3′ orientation as the functional nucleic acid molecule. Alternatively, “inverted” refers to the situation in which the regulatory sequence is 3′ to 5′ oriented relative to the functional nucleic acid molecule.

In a further embodiment, the regulatory sequence is located 3′ of the two or more target binding sequences within the functional nucleic acid molecule.

### SINE B2

In one embodiment, the regulatory sequence comprises a SINE B2 element or a functionally active fragment thereof.

The SINE B2 element is preferably in an inverted orientation relative to the 5′ to 3′ orientation of the functional nucleic acid molecule, i.e. an inverted SINE B2 element.

The regulatory sequence comprises a SINE B2 element, or a functionally active fragment thereof. Said sequence enhances translation of the target mRNA sequence.

In one embodiment, the regulatory sequence consists of a SINE B2 element or a functionally active fragment of a SINE B2 element.

The SINE B2 element, or functionally active fragment thereof, may be in the direct or inverted orientation relative to the functional nucleic acid molecule.

In one embodiment the regulatory sequence comprises a SINE B2 element or a functionally active fragment thereof, wherein the regulatory sequence is orientated, within the functional nucleic acid molecule, in the direct orientation relative to the 5′ to 3′ orientation of the functional nucleic acid molecule.

In one embodiment the regulatory sequence comprises a SINE B2 element or a functionally active fragment thereof, wherein the regulatory sequence is orientated, within the functional nucleic acid molecule, in the inverted orientation relative to the 5′ to 3′ orientation of the functional nucleic acid molecule

The term “SINE” (Short Interspersed Nuclear Element) refers to an interspersed repetitive sequence: (a) that encodes a protein having neither reverse-transcription activity nor endonuclease activity or the like, and (b) whose complete or incomplete copy sequences exist abundantly in genomes of living organisms.

The term “SINE B2 element” is defined in WO 2012/133947, where specific examples are also provided (see table starting on page 69 of the PCT publication) which is incorporated herein by reference in its entirety. The term is intended to encompass both SINE B2 elements in direct orientation and in inverted orientation relative to the 5′ to 3′ orientation of the functional nucleic acid molecule.

SINE B2 elements may be identified, for example, using programs like RepeatMask as published (Bedell et al. Bioinformatics. 2000 November; 16(11): 1040-1. MaskerAid: a performance enhancement to RepeatMasker). A sequence may be recognizable as a SINE B2 element by returning a hit in a Repbase database with respect to a consensus sequence of a SINE B2, with a Smith-Waterman (SW) score of over 225, which is the default cutoff in the RepeatMasker program. Generally, a SINE B2 element is not less than 20 bp and not more than 400 bp. Preferably, the SINE B2 is derived from tRNA.

By the term “functionally active fragment of a SINE B2 element” there is intended a portion of sequence of a SINE B2 element that retains protein translation enhancing activity. This term also includes sequences that are mutated in one or more nucleotides with respect to the wild-type sequences, but retain protein translation enhancing activity. The term is intended to encompass both SINE B2 elements in direct orientation and in inverted orientation relative to the 5′ to 3′ orientation of the functional nucleic acid molecule.

Short fragments of the regulatory sequence (such as a SINE B2 element) are particularly useful when providing functional RNA molecules for use as a nucleic acid therapeutic. RNA molecules are highly unstable in living organisms, therefore stability provided by the chemical modifications as described herein, is more effective for shorter RNA molecules. Therefore, in one embodiment, the regulatory sequence comprises a functionally active fragment that is less than 250 nucleotides, such as less than 240 nucleotides, less than 230 nucleotides, less than 220 nucleotides, less than 210 nucleotides, less than 200 nucleotides, less than 190 nucleotides, less than 180 nucleotides, less than 170 nucleotides, less than 160 nucleotides, less than 150 nucleotides, less than 140 nucleotides, less than 130 nucleotides, less than 120 nucleotides, less than 110 nucleotides, less than 100 nucleotides, less than 90 nucleotides, less than 80 nucleotides, less than 70 nucleotides, less than 60 nucleotides, less than 50 nucleotides, less than 40 nucleotides, less than 30 nucleotides, less than 20 nucleotides, less than 10 nucleotides.

In some embodiments the regulatory sequence comprises or consists of a functionally active fragment of a sequence selected from the group consisting of SEQ ID NOs 2-54, wherein the fragment is about is about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250 or more nucleotides in length.

In one embodiment, the regulatory sequence comprises or consists of a functionally active fragment of a sequence selected from the group consisting of SEQ ID NOs 2-54, wherein the fragment is 187 nucleotides in length.

In one embodiment, the regulatory sequence comprises or consists of a functionally active fragment of a sequence selected from the group consisting of SEQ ID NOs 2-54, wherein the fragment is 183 nucleotides in length.

In one embodiment, the regulatory sequence comprises or consists of a functionally active fragment of a sequence selected from the group consisting of SEQ ID NOs 2-54, wherein the fragment is 167 nucleotides in length.

In one embodiment, the regulatory sequence comprises or consists of a functionally active fragment of a sequence selected from the group consisting of SEQ ID NOs 2-54, wherein the fragment is 77 nucleotides in length.

In one embodiment, the regulatory sequence comprises or consists of a functionally active fragment of a sequence selected from the group consisting of SEQ ID NOs 2-54, wherein the fragment is 38 nucleotides in length.

In one embodiment, the regulatory sequence comprises or consists of a functionally active fragment of a sequence selected from the group consisting of SEQ ID NOs 2-54, wherein the fragment is 29 nucleotides in length.

In one embodiment, the functional nucleic acid molecule comprises a SINE B2 element, wherein said SINE B2 element comprises a sequence selected from the group consisting of SEQ ID NOs 2-54, or a functionally active fragment thereof.

In one embodiment, the functional nucleic acid molecule comprises a SINE B2 element, wherein said SINE B2 element consists of a sequence selected from the group consisting of SEQ ID NOs 2-54, or a functionally active fragment thereof.

In one embodiment, the functional nucleic acid molecule comprises a SINE B2 element, wherein said SINE B2 element comprises or consists of a sequence which has at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to a sequence selected from the group consisting of SEQ ID NOs 2-54, or a functionally active fragment thereof.

Preferably, the regulatory sequence comprises a sequence with at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to a sequence selected from the group consisting of SEQ ID NO: 2-54.

In one embodiment, the regulatory sequence consists of a sequence with at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to a sequence selected from the group consisting of SEQ ID NO: 2-54.

In one embodiment, the regulatory sequence comprises a functionally active fragment of a SINE B2 element according to the foregoing, wherein the fragment comprises a sequence with at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to a fragment or region of a sequence selected from the group consisting of SEQ ID NO: 2-54.

In one embodiment, the regulatory sequence consists of a functionally active fragment of a SINE B2 element according to the foregoing, wherein the fragment comprises or consists of a sequence with at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to a sequence selected from the group consisting of SEQ ID NO: 2-54.

SEQ ID NO: 2 (the 167 nucleotide variant of the inverted SINE B2 element in AS Uchl1) and SEQ ID NO: 3 (the 77 nucleotide variant of the inverted SINE B2 element in AS Uchl1 that includes nucleotides 44 to 120), as well as sequences with a suitable percentage identity (e.g., at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity) to these sequences are particularly preferred.

Other inverted SINE B2 elements and functionally active fragments of inverted SINE B2 elements are SEQ ID NO: 4-54. Experimental data showing the protein translation enhancing activity of these sequences is not explicitly shown in the present patent application, but is disclosed in e.g., WO 2019/150346, which is incorporated herein by reference in its entirety. SEQ ID NO: 4-54 can therefore also be used as regulatory sequences in the functional nucleic acid molecule of the present invention.

SEQ ID NO: 4-7, 9-12 and 19 are functionally active fragments of inverted SINE B2 transposable element derived from AS Uchl1. The use of functional fragments reduces the size of the regulatory sequence, which is advantageous if used in an expression vector (e.g. viral vectors which may be size-limited) because this provides more space for the two or more target sequences and/or expression elements.

SEQ ID NO: 8 is a full-length 183 nucleotide (nt) inverted SINE B2 transposable element derived from AS Uchl1. SEQ ID NO: 13-18, 20, 21, 40-43 are mutated functionally active fragments of inverted SINE B2 transposable element derived from AS Uchl1.

SEQ ID NO: 22-26, 29-39 are different SINE B2 transposable elements. SEQ ID NO: 27 and 28 are sequences in which multiple inverted SINE B2 transposable element have been inserted.

In one embodiment the SINE B2 fragment is about 10, about 20, about 29, about 30, about 38, about 40, about 50, about 60, about 70, about 77, about 80, about 90, about 100, about 110, about 120, about 130, about 140, about 150, about 160, about 167, about 170, about 180, about 183, about 190, about 200, about 210, about 220, about 230, about 240, about 250 or more nucleotides in length.

### IRES

Alternatively, the regulatory sequence comprises an IRES sequence, or functionally active fragment thereof. The regulatory sequence may also comprise a fragment, such as a functionally active fragment, of an IRES sequence. Therefore, in one embodiment, the regulatory sequence comprises an IRES sequence or a functionally active fragment of an IRES sequence. Said sequence enhances translation of the target mRNA.

The regulatory sequence comprises an IRES sequence, or a functionally active fragment thereof. Said sequence enhances translation of the target mRNA sequence.

The terms “internal ribosome entry site (IRES) sequence” is defined in WO 2019/058304, which is incorporated herein by reference in its entirety. IRES sequences recruit the 40S ribosomal subunit and promote cap-independent translation of a subset of protein coding mRNAs. IRES sequences are generally found in the 5′ untranslated region (5′UTR) of cellular mRNAs coding for stress-response genes, thus stimulating their translation in cis.

The person skilled in the art would know that an IRES sequence is a nucleotide sequence capable of promoting translation of a second cistron in a bicistronic construct. Typically, a dual luciferase (Firefly luciferase [Fluc], Renilla Luciferase [Rluc]) encoding plasmid is used for experimental tests. Said test may be considered “The Standard Bicistronic Plasmid Test for Cellular mRNA IRESs” used to test putative IRES sequences. The foregoing is a functional test wherein the putative IRES sequence is inserted between RLuc and FLuc, e.g., as described in Jasckson, Cold Spring Harb Perspect Biol 2013; 5:a011569, wherein the translational function of the putative IRES sequence is determined by the Fluc/RLuc value, thus measuring cis-acting activity.

The regulatory sequence of the functional nucleic acid molecule of the invention may be trans-acting. Thus, in one embodiment the functional nucleic acid molecule comprises an IRES regulatory sequence that is trans-acting.

A major database exists, namely IRESite, for the annotation of nucleotide sequences that have been experimentally validated as IRES, using dual reporter or bicistronic assays (http://iresite.org/IRESite_web.php).

Within the IRESite, a web-based tool is available to search for sequence-based and structure-based similarities between a query sequence of interest and the entirety of annotated and experimentally validated IRES sequences within the database. The output of the program is a probability score for any nucleotide sequence to be able to act as IRES in a validation experiment with bicistronic constructs. Additional sequence-based and structure-based web-based browsing tools are available to suggest, with a numerical predicting value, the IRES activity potentials of any given nucleotide sequence (http://rna.informatik.uni-freiburg.de/; http://regrna.mbc.nctu.edu.tw/index1.php).

Several IRESs having sequences ranging from 48 to 576 nucleotides have been tested with success, e.g. human Hepatitis C Virus (HCV) IRESs (e.g. SEQ ID NO: 55 and 56), human poliovirus IRESs (e.g. SEQ ID NO: 57 and 58), human encephalomyocarditis (EMCV) virus (e.g. SEQ ID NO: 59 and 60), human cricket paralysis (CrPV) virus (e.g. SEQ ID NO: 61 and 62), human Apaf-1 (e.g. SEQ ID NO: 63 and 64), human ELG-1 (e.g. SEQ ID NO: 65 and 66), human c-MYC (e.g. SEQ ID NO: 67-70) and human dystrophin (DMD) (e.g. SEQ ID NO: 71 and 72).

In some embodiments the regulatory sequence comprises a sequence selected from the group consisting of SEQ ID NOs 55-72, or a functionally active fragment thereof.

In some embodiments the regulatory sequence consists of a sequence selected from the group consisting of SEQ ID NOs 55-72, or a functionally active fragment thereof.

Such sequences have been disclosed, defined and exemplified in WO 2019/058304, which is incorporated herein by reference in its entirety.

In one embodiment the regulatory element has at least about 75% sequence identity, at least about 80% sequence identity, at least about 85% sequence identity, at least about 86% sequence identity, at least about 87% sequence identity, at least about 88% sequence identity, at least about 89% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or 100% sequence identity to any one of SEQ ID NOs 55-72.

In one embodiment, the at least one regulatory sequence consists of a sequence with at least about 75% sequence identity, at least about 80% sequence identity, at least about 85% sequence identity, at least about 86% sequence identity, at least about 87% sequence identity, at least about 88% sequence identity, at least about 89% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or 100% sequence identity to a sequence selected from the group consisting of SEQ ID NOs 55-7072

In some embodiments the regulatory sequence comprises or consists of a functionally active fragment of any one of SEQ ID NOs 55-72, wherein the fragment is about is about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370 or more nucleotides in length.

In some embodiments, the functionally active fragment retains IRES activity within the definition provided above.

In some embodiments, the functionally active fragment retains protein translation enhancing activity.

It will be understood that, owing to the functional nature of The Standard Bicistronic Plasmid Test for Cellular mRNA IRESs, a “functionally active fragment” of an IRES might also be considered an IRES perse. Herein, “functionally active fragment” of an IRES is utilised to delineate IRES sequences that are shorter in length as compared with ‘parental’ IRES sequences from which they are designed or derived.

TABLE 2
Exemplary regulatory sequences

| SEQ ID | |
|---|---|
| NO: | Exemplary SINE B2 sequence |
| 2 | cagugcuaga ggaggucaga agagggcauu ggauccccca gaacuggagu |
| | uauacgguaa ccucguggug guugugaacc accaugugga uggauauuga |
| | guuccaaaca cugguccugu gcaagagcau ccagugcucu uaagugcuga |
| | gccaucucuu uagcucc |
| 3 | gaacuggagu uauacgguaa ccucguggug guugugaacc accaugugga |
| | uggauauuga guuccaaaca cuggucc |
| 4 | ccucguggug guugugaacc accaugugg |
| 5 | guuauacggu aaccucgugg ugguugugaa ccaccaugug gauggauauu |
| | gaguuccaaa |
| 6 | aucccccaga acuggaguua uacgguaacc ucgugguggu ugugaaccac |
| | cauguggaug gauauugagu uccaaacacu gguccugugc aagagcau |
| 7 | gaagagggca uuggaucccc cagaacugga guuauacggu aaccucgugg |
| | ugguugugaa ccaccaugug gauggauauu gaguuccaaa cacugguccu |
| | gugcaagagc auccagugcu cuuaagugc |
| 8 | gggcagugcu agaggagguc agaagagggc auuggauccc ccagaacugg |
| | aguuauacgg uaaccucgug gugguuguga accaccaugu ggauggauau |
| | ugaguuccaa acacuggucc ugugcaagag cauccagugc ucuuaagugc |
| | ugagccaucu cuuuagcucc agucucuuaa gcu |
| 9 | gaacuggagu uauacgguaa ccucguggug guugugaacc accaugugga |
| | uggauauuga guuccaa |
| 10 | ggauccccca gaacuggagu uauacgguaa ccucguggug guugugaacc |
| | accaugugga uggauauuga guuccaaaca cugguccugu gcaagagcau |
| | ccagugc |
| 11 | agagggcauu ggauccccca gaacuggagu uauacgguaa ccucguggug |
| | guugugaacc accaugugga uggauauuga guuccaaaca cugguccugu |
| | gcaagagcau ccagugcucu uaagugc |
| 12 | gguaaccucg uggugguugu gaaccaccau guggaugg |
| 13 | cagugcuaga ggaggucaga agagggcauu ggauccccca gaacuggagu |
| | uauacgauaa ccucguggug guugugaacc accaugugga uggauauuga |
| | guuccaaaca cugguccugu gcaagagcau ccagugcucu uaagugcuga |
| | gccaucucuu uagcuccagu cucuuaagcu |
| 14 | cagugcuaga ggaggucaga agagggcauu ggauccccca gaacuggagu |
| | uauacgcuaa ccucguggug guugugaacc accaugugga uggauauuga |
| | guuccaaaca cugguccugu gcaagagcau ccagugcucu uaagugcuga |
| | gccaucucuu uagcuccagu cucuuaagcu |
| 15 | cagugcuaga ggaggucaga agagggcauu ggauccccca gaacuggcgu |
| | uauacgguaa ccucguggug guugugaacc accaugugga uggauauuga |
| | guuccaaaca cugguccugu gcaagagcau ccagugcucu uaagugcuga |
| | gccaucucuu uagcuccagu cucuuaagcu |
| 16 | cagugcuaga ggaggucaga agagggcauu ggauccccca gaaguggagu |
| | uauacgguaa ccucguggug guugugaacc accaugugga uggauauuga |
| | guuccaaaca cugcuccugu gcaagagcau ccagugcucu uaagugcuga |
| | gccaucucuu uagcuccagu cucuuaagcu |
| 17 | cagugcuaga ggaggucaga agagggcauu ggauccccca gauggugagu |
| | uauacgguaa ccucguggug guugugaacc accaugugga uggauauuga |
| | guuccaaaca cgucaccugu gcaagagcau ccagugcucu uaagugcuga |
| | gccaucucuu uagcuccagu cucuuaagcu |
| 18 | cagugcuaga ggaggucaga agagggcauu ggauccccca gaacugcacu |
| | auacgguaac cucguggugg uugugaacca ccauguggau ggauauugag |
| | uuccaaauga gugguccugu gcaagagcau ccagugcucu uaagugcuga |
| | gccaucucuu uagcuccagu cucuuaagcu |
| 19 | gggcauugga ucccccagaa auauugaguu ccaaacacug guccugugca |
| | gugaaccacc auguggaugg cuggaguuau acgguaaccu cguggugguu |
| | agagcaucca gugcuc |
| 20 | ggacuggagu uauacgguaa ccucguggug guugugaacc accaugugga |
| | uggauauuga guuccaaaca cuggucc |
| 21 | gaacuggcgu uauacgguaa ccucguggug guugugaacc accaugugga |
| | uggauauuga guuccaaaca cuggucc |
| 22 | gaugccuuag aaguggaguu aagaguugug agcugccguu uuuugguucu |
| | gggacucgaa cucguuuccu cugauacuau caaccaccaa gccaucucuu |
| | cagcccc |
| 23 | gccagaagaa guugugggau ucccuggaac uggagcaacc aacaguuugu |
| | gugcaccaug uggguaaugg gaaucgaacc uggguccucu auaagacugg |
| | ccagugcucu uaacuacuga ggugcauuuc u |
| 24 | uuauuuuaaa uauaugagua uuucaccugc auaggcgcac aguacccaca |
| | gagacuagaa gaggguggca gaucuccuga gacuggaguu aaugcuugug |
| | agcugccaug uggaugcugg aaaucaaacc cagguccuuu ggaaggcagg |
| | caggugcucu uaaucaugga agcaucucuu cagcucc |
| 25 | cagcgacauc agaagaggau auuggauccc auuacagaug guugaaggcc |
| | accaugucgu ugcugggaau gaacucaaga ccucuggaag agcagucagu |
| | gcucuuaacc ucugagccau cucuccagcc c |
| 26 | auccccucca aagcucaaga ugguuguaag ccacccugug auugcuggga |
| | uuugaacuca agaccuccgg aagagcaauu agugcucuua accgcugagc |
| | aaucucucca gccc |
| 27 | gugcagugcu agaggagguc agaagagggc auuggauccc ccagaacugg |
| | aguuauacgg uaaccucgug gugguuguga accaccaugu ggauggauau |
| | ugaguuccaa acacuggucc ugugcaagag cauccagugc ucuuaagugc |
| | ugagccaucu cuuuagcucc uuauuuuaaa uauaugagua uuucaccugc |
| | auaggcgcac aguacccaca gagacuagaa gaggguggca gaucuccuga |
| | gacuggaguu aaugcuugug agcugccaug uggaugcugg aaaucaaacc |
| | cagguccuuu ggaaggcagg caggugcucu uaaucaugga agcaucucuu |
| | cagcucc |
| 28 | gugcagugcu agaggagguc agaagagggc auuggauccc ccagaacugg |
| | aguuauacgg uaaccucgug gugguuguga accaccaugu ggauggauau |
| | ugaguuccaa acacuggucc ugugcaagag cauccagugc ucuuaagugc |
| | ugagccaucu cuuuagcucc gugcgaauuc ggugcagugc uagaggaggu |
| | cagaagaggg cauuggaucc cccagaacug gaguuauacg guaaccucgu |
| | ggugguugug aaccaccaug uggauggaua uugaguucca aacacugguc |
| | cugugcaaga gcauccagug cucuuaagug cugagccauc ucuuuagcuc |
| | cgugcgaauu cggugcagug cuagaggagg ucagaagagg gcauuggauc |
| | ccccagaacu ggaguuauac gguaaccucg uggugguugu gaaccaccau |
| | guggauggau auugaguucc aaacacuggu ccugugcaag agcauccagu |
| | gcucuuaagu gcugagccau cucuuuagcu cc |
| 29 | uuuuuuuaaa aauuuauuuu uauuuuaugu guaugagugu uuugccugua |
| | uguaugucug uguaccacgu gcgugccugg ugcccgcgga ggccagaaga |
| | gggcgucgga uccccuggaa cuggaguuac agaugguugu gagccgccau |
| | gugggugcug ggaaucgaac ccggguccuc uggaagagca gccagugcuc |
| | uuaaccgcug agccaucucu ccagcccc |
| 30 | uuuuuuuuac uuguauaggu guuuugccug cauguguauc uaucuaugua |
| | ccgaauaugu uccugguauc cacagagacc aaaaguggau guuguaucuc |
| | cugaaauugg agucauagac aguuaugagc ugccauuuga gugcuuggaa |
| | uagaacccag guccucuuaa agagcaucca gugcucuuaa aaacugagac |
| | aucucuguag ccuc |
| 31 | uuuauuuugc uuuauguguc ugaguguuug cuugaaugua ugucugugua |
| | ccacgccugu accuugugcc uucagaguug agaggagggc auaggaucuc |
| | cuggaacugg aauugcaggu gguugugagc cacccugugg guccugggga |
| | ccauacucca gcaagaacau caugugcucu uaauuccuga gucuccaacc |
| 32 | uuuauuuacu uaucuuuaug uguaugagug uguugucaga cuguuauguc |
| | ugugugucac augcaugccu gcuguucaug gaguccagaa gagggcaucg |
| | gauccccugg aacuggaguu acagaugagu ggccauguga auguuaagaa |
| | ccaaaccugg guccucugaa agagcagaca augcucuuaa cuacugagcu |
| | gucucuccag cccc |
| 33 | uuauuuuauu cguguaagug uuuugccagc aucuaugucu ucgcacuaug |
| | ugcaggucug gugccugagg gguccagacg agagcacugg gucuccggga |
| | acuggaguua cagaucauug ugagccacca ugugggugca gggaaucgaa |
| | ccugggaccu cuggaggagc agccacugcu cuuaaccacu acacuauuuc |
| | uccag |
| 34 | ucuguggacc acuguguaca gaagccugag aaggcuagca gauccccaga |
| | acuggaacug ugagacgcug ugcuauggag gugcuaggaa cugaaaaugg |
| | auggguccuc ugcaagagca g |
| 35 | uuguuuuaau ugaauggcua uaggguguuu cuucuguaug uauaucuaug |
| | uuugguaccu acagaggcau cagauccucu ggaacuguag uugcugacag |
| | uugugagcug ucauggggau gcuggaauug aaccuggauc cuaugaaaga |
| | acagccagug uucuuaaccg cugagcuauc ucuccaggcc c |
| 36 | uuuuuuuuuu aauuuuaaaa aaaaagauuu uauuuauuua uuuuauauau |
| | gaugaguaca cugucacucu uuucagacac ccuagaaaag gggggcauca |
| | gaucccauua cagaugguug ugagccacau gguugcuggg aauugaccuc |
| | aggaccucug aaagagcagu cagugcucuc aaccuuugag ucaucucucc |
| | agccc |
| 37 | auguauaucu guaaugggac auacucacau acaugggcac gugaguauaa |
| | aaggccagaa gagagcacug gacccucugg aguugagauu cuaagcaguu |
| | gugaaccauc ugauguaggu gcugggaacu gaacuugggu ccuuugcuag |
| | agaaguaugu cucuuaacca cugagccgua ucuccauccc |
| 38 | uaaagauuua uucauuaagu acacuguagc uaucuucaga cgcaucagaa |
| | gagggcguca gaucucuuua caggugguug ugagccacca ugugguugcu |
| | ggaauuugaa cucaggaccu ucaaaagagc agucaguguu cuuaaccgcu |
| | gagccaucuc uccaacccc |
| 39 | uuauuuauua uaaguacacu guagcugucu ucagacacaa caaaagaggg |
| | cgucagaucu cauuacaggu gguugagcca ccaugugguu gcugggauuu |
| | gaacucagga ccuucagaac agucagugcu cuuacccacu gagccagcga |
| | gccagcccc |
| 40 | gaacuggagu uauacgguaa ccucguggug guugggaacc accaugugga |
| | uggauauuga guuccaaaca cuggucc |
| 41 | uggauauuga guuccaaaca cuggucc guucccaacc accaugugga |
| | gaacuggagu uauacgguaa ccucguggug |
| 42 | ggaccggagu uauacgguaa ccgcguggug guugugaacc accacgcgga |
| | uggauauuga guuccaaaca ccggucc |
| 43 | gaacuagagu uauacgguaa ccacauggug guugugaacc accaugugga |
| | uggauauuga guuccaaaca cuaguuc |
| 44 | uuauuuuaaa uauaugagua uuucaccugc auaggcgcac aguacccaca |
| | gagacuagaa gaggguagua gauccccuag aacuggaguu auacgguaac |
| | cucguggugg uugugagcua ccauguggau ggauacuggg aaucaaaccc |
| | agguccugug gaaggcaggc aggugcucuc aagcacugag ccaucucuuc |
| | agcucc |
| 45 | uuauuuuaaa uauaugagua uuucaccugc auaggcgcac agugcucaag |
| | gagaucagaa gagggcauca gaucuccuga gacuggaguu auacgguaac |
| | cucgugaugg uugugaacua ccauguggau ggauauugag uuccaaacac |
| | agguccugug caagagcagc aggugcucuu aagcacggaa ccaucucuuu |
| | agcucc |
| 46 | gaggcuagaa gaggguauca gauccccuga gacuggaguu auacgguaac |
| | cucguggugg uugugagcca ccauguggau ggauacugag aaccaaaccc |
| | ugguccugug caagagcauc aggugcucuu aagcacggaa ccaucucuuc |
| | agcucc |
| 47 | guccugugca agagcaucga acucggugcu cuuaagcaca gaagccacca |
| | agccaucucu ucagcccc |
| 48 | cagugcuaga ggaggucaga agagggcauc ccccagccuc guggugguug |
| | ugaaccacca uguggcugug caagagcaug cucuuaagug cugagccauc |
| | ucuuuagcuc |
| 49 | gagggcauug gaucccccag aacuggaguu auacgguaac cucguggugg |
| | uugugaacca ccauguggau ggauauugag uuccaaacac ugguccugug |
| | caagagcauc cagugcucuu aagugc |
| 50 | ggauccccca gaacuggagu uauacgguaa ccucguggug guugugaacc |
| | accaugugga uggauauuga guuccaaaca cugguccug |
| 51 | ugcuagagga ggucagaaga gggcauugga ugcaaaucca gugcucuuaa |
| | gugcugagcc aucucuuuag cu |
| 52 | gagggcauug gaucccccag aacuggaguu auacgguaac gauggauauu |
| | gaguuccaaa cacugguccu gugcaagagc auccagugcu cuua |
| 53 | CAGTGCTAGAGGAGGTCAGAAGAGGGCATTGGATCCCCCAGAACTGGAGTTATACG |
| | GTAACCTCGTGGTGGTTGTGAACCACCATGTGGATGGATATTGAGTTCCAAACACT |
| | GGTCCTGTGCAAGAGCATCCAGTGCTCTTAAGTGCTGAGCCATCTCTTTAGCTCCA |
| | GTCTCTTAAAAAACAAACAAACGAACGAACAGCAAGGGAGCTGGGTATGACAACAC |
| | ATACTATAATTCTAGTACTCAGGATGCTGAAACAGGAGGATTGCCTGACTGGGAGA |
| | TATAAGGAGAATCTGTTGTCACCCCCACCCCTCCCCATAAAGGCAGAATAAAAGAA |
| | CGTCCTATAAACAAATAAACAAACAACCCAATAAAACAAAACCAAGATCTCTCCAC |
| | CTTTTCTTTGCTTTTTCAGACTTTGTAATAAGGCCCTTTGGAGTGCAGGATATTCG |
| | GCAGGACAAGCAGAGAGGGAGACCATCAGTTCTTTCTTTGATCAAGAAGACTATGT |
| | TCCTTAGCAAACTGGTGTGTATTATCTCTTATGCAATGAGCCTGGAAAGAGGGCAC |
| | AGCCACCGAGGATGGTACAGCATGGATGGATGGTACGCTACAGAGACTCGGGAGCC |
| | CAACTGTGAGTGGCTGACTGGCATGGTAGGTTCAGGGAAGAATTGGCCTGTGAAGA |
| | AAATGTTCTTGAAAAGTGAACAAGGTGCAGGAGGTAGGAGTGGGTCCTGGGCAAAG |
| | CAGGGGGTGCATCCCAGCCTCAGGGAATAGCACAGCAGAGGTCTGTTGATGCATGC |
| | GAGTGCATGACCTGCTTGCCAATAGACGATCAAGAATGGGCAAAGCATCATGGGTG |
| | ATGAGTGGGAGAGGGGATGAGACATTCCTTTCTCCCTGCTGAGACTTCCATTGAAC |
| | CGATGAGTTCTGAATAGAAGATGCCCCCCCACCCCCCCACCAGTGTAGAATCTGAA |
| | GGGAGGCATATATTACCCTATATTACTCTGTGTTGGCGGCGAGCTATCTGACAGCC |
| | AACCTTCCCATACATTTCATTGGGCATACACTAATGACAGGAAGTTCCTTTTGCTT |
| | GTATGCAAGAGATGGCTCACACGATGGAGAATTTAATCTTGAAGGGC |
| 54 | GATGCGGCCGCCACTGTGCTGGATATCTGCAGAATTCGCCCTTCAGTGCTAGAGGA |
| | GGTCAGAAGAGGGCATTGGATCCCCCAGAACTGGAGTTATACGGTAACCTCGTGGT |
| | GGTTGTGAACCACCATGTGGATGGATATTGAGTTCCAAACACTGGTCCTGTGCAAG |
| | AGCATCCAGTGCTCTTAAGTGCTGAGCCATCTCTTTAGCTCCAGTCTCTTAAAAAA |
| | CAAACAAACGAACGAACAGCAAGGGAGCTGGGTATGACAACACATACTATAATTCT |
| | AGTACTCAGGATGCTGAAACAGGAGGATTGCCTGACTGGGAGATATAAGGAGAATC |
| | TGTTGTCACCCCCACCCCTCCCCATAAAGGCAGAATAAAAGAACGTCCTATAAACA |
| | AATAAACAAACAACCCAATAAAACAAAACCAAGATCTCTCCACCTTTTCTTTGCTT |
| | TTTCAGACTTTGTAATAAGGCCCTTTGGAGTGCAGGATATTCGGCAGGACAAGCAG |
| | AGAGGGAGACCATCAGTTCTTTCTTTGATCAAGAAGACTATGTTCCTTAGCAAACT |
| | GGTGTGTATTATCTCTTATGCAATGAGCCTGGAAAGAGGGCACAGCCACCGAGGAT |
| | GGTACAGCATGGATGGATGGTACGCTACAGAGACTCGGGAGCCCAACTGTGAGTGG |
| | CTGACTGGCATGGTAGGTTCAGGGAAGAATTGGCCTGTGAAGAAAATGTTCTTGAA |
| | AAGTGAACAAGGTGCAGGAGGTAGGAGTGGGTCCTGGGCAAAGCAGGGGGTGCATC |
| | CCAGCCTCAGGGAATAGCACAGCAGAGGTCTGTTGATGCATGCGAGTGCATGACCT |
| | GCTTGCCAATAGACGATCAAGAATGGGCAAAGCATCATGGGTGATGAGTGGGAGAG |
| | GGGATGAGACATTCCTTTCTCCCTGCTGAGACTTCCATTGAACCGATGAGTTCTGA |
| | ATAGAAGATGCCCCCCCACCCCCCCACCAGTGTAGAATCTGAAGGGAGGCATATAT |
| | TACCCTATATTACTCTGTGTTGGCGGCGAGCTATCTGACAGCCAACCTTCCCATAC |
| | ATTTCATTGGGCATACACTAATGACAGGAAGTTCCTTTTGCTTGTATGCAAGAGAT |
| | GGCTCACACGATGGAGAATTTAATCTTGAAGGGC |
| SEQ ID | |
| NO: | Exemplary IRES Sequence |
| 55 | gccagccccc ugaugggggc gacacuccac caugaaucac uccccuguga |
| | ggaacuacug ucuucacgca gaaagcgucu agccauggcg uuaguaugag |
| | ugucgugcag ccuccaggac ccccccuccc gggagagcca uaguggucug |
| | cggaaccggu gaguacaccg gaauugccag gacgaccggg uccuuucuug |
| | gauaaacccg cucaaugccu ggagauuugg gcgugccccc gcaagacugc |
| | uagccgagua guguuggguc gcgaaaggcc uugugguacu gccugauagg |
| | gugcuugcga gugccccggg aggucucgua gaccgugcac caugagcacg |
| | aauccuaaac cucaaagaaa aaccaaacgu aac |
| 56 | guuacguuug guuuuucuuu gagguuuagg auucgugcuc auggugcacg |
| | gucuacgaga ccucccgggg cacucgcaag cacccuauca ggcaguacca |
| | caaggccuuu cgcgacccaa cacuacucgg cuagcagucu ugcgggggca |
| | cgcccaaauc uccaggcauu gagcggguuu auccaagaaa ggacccgguc |
| | guccuggcaa uuccggugua cucaccgguu ccgcagacca cuauggcucu |
| | cccgggaggg gggguccugg aggcugcacg acacucauac uaacgccaug |
| | gcuagacgcu uucugcguga agacaguagu uccucacagg ggagugauuc |
| | augguggagu gucgccccca ucagggggcu ggc |
| 57 | augagucugg acaucccuca ccggugacgg ugguccaggc ugcguuggcg |
| | gccuaccuau ggcuaacgcc augggacgcu aguugugaac aaggugugaa |
| | gagccuauug agcuacauaa gaauccuccg gccccugaau gcggcuaauc |
| | ccaaccucgg agcagguggu cacaaaccag ugauuggccu gucguaacgc |
| | gcaaguccgu ggcggaaccg acuacuuugg guguccgugu uuccuuuuau |
| | uuuauugugg cugcuuaugg ugacaaucac agauuguuau cauaaagcga |
| | auuggauugg cc |
| 58 | ggccaaucca auucgcuuua ugauaacaau cugugauugu caccauaagc |
| | agccacaaua aaauaaaagg aaacacggac acccaaagua gucgguuccg |
| | ccacggacuu gcgcguuacg acaggccaau cacugguuug ugaccaccug |
| | cuccgagguu gggauuagcc gcauucaggg gccggaggau ucuuauguag |
| | cucaauaggc ucuucacacc uuguucacaa cuagcguccc auggcguuag |
| | ccauagguag gccgccaacg cagccuggac caccgucacc ggugagggau |
| | guccagacuc au |
| 59 | cccccccucu cccucccccc ccccuaacgu uacuggccga agccgcuugg |
| | aauaaggccg gugugcguuu gucuauaugu uauuuuccac cauauugccg |
| | ucuuuuggca augugagggc ccggaaaccu ggcccugucu ucuugacgag |
| | cauuccuagg ggucuuuccc cucucgccaa aggaaugcaa ggucuguuga |
| | augucgugaa ggaagcaguu ccucuggaag cuucuugaag acaaacaacg |
| | ucuguagcga cccuuugcag gcagcggaac cccccaccug gcgacaggug |
| | ccucugcggc caaaagccac guguauaaga uacaccugca aaggcggcac |
| | aaccccagug ccacguugug aguuggauag uuguggaaag agucaaaugg |
| | cucuccucaa gcguauucaa caaggggcug aaggaugccc agaagguacc |
| | ccauuguaug ggaucugauc uggggccucg gugcacaugc uuuacaugug |
| | uuuagucgag guuaaaaaac gucuaggccc cccgaaccac ggggacgugg |
| | uuuuccuuug aaaaacacga ugauaa |
| 60 | uuaucaucgu guuuuucaaa ggaaaaccac guccccgugg uucggggggc |
| | cuagacguuu uuuaaccucg acuaaacaca uguaaagcau gugcaccgag |
| | gccccagauc agaucccaua caauggggua ccuucugggc auccuucagc |
| | cccuuguuga auacgcuuga ggagagccau uugacucuuu ccacaacuau |
| | ccaacucaca acguggcacu gggguugugc cgccuuugca gguguaucuu |
| | auacacgugg cuuuuggccg cagaggcacc ugucgccagg ugggggguuc |
| | cgcugccugc aaagggucgc uacagacguu guuugucuuc aagaagcuuc |
| | cagaggaacu gcuuccuuca cgacauucaa cagaccuugc auuccuuugg |
| | cgagagggga aagaccccua ggaaugcucg ucaagaagac agggccaggu |
| | uuccgggccc ucacauugcc aaaagacggc aauauggugg aaaauaacau |
| | auagacaaac gcacaccggc cuuauuccaa gcggcuucgg ccaguaacgu |
| | uagggggggg ggagggagag gggggg |
| 61 | aaagcaaaaa ugugaucuug cuuguaaaua caauuuugag agguuaauaa |
| | auuacaagua gugcuauuuu uguauuuagg uuagcuauuu agcuuuacgu |
| | uccaggaugc cuaguggcag ccccacaaua uccaggaagc ccucucugcg |
| | guuuuucaga uuagguaguc gaaaaaccua agaaauuuac cu |
| 62 | agguaaauuu cuuagguuuu ucgacuaccu aaucugaaaa accgcagaga |
| | gggcuuccug gauauugugg ggcugccacu aggcauccug gaacquaaag |
| | cuaaauagcu aaccuaaaua caaaaauagc acuacuugua auuuauuaac |
| | cucucaaaau uguauuuaca agcaagauca cauuuuugcu uu |
| 63 | cagagaucca ggggaggcgc cugugaggcc cggaccugcc ccggggcgaa |
| | ggguaugugg cgagacagag cccugcaccc cuaauucccg guggaaaacu |
| | ccuguugccg uuucccucca ccggccugga gucucccagu cuugucccgg |
| | cagugccgcc cuccccacua agaccuaggc gcaaaggcuu ggcucauggu |
| | ugacagcuca gagagagaaa gaucugaggg a |
| 64 | ucccucagau cuuucucucu cugagcuguc aaccaugagc caagccuuug |
| | cgccuagguc uuagugggga gggcggcacu gccgggacaa gacugggaga |
| | cuccaggccg guggagggaa acggcaacag gaguuuucca ccgggaauua |
| | ggggugcagg gcucugucuc gccacauacc cuucgccccg gggcaggucc |
| | gggccucaca ggcgccuccc cuggaucucu g |
| 65 | acuuuuggug ggcauuuaaa aaugugugug uauguguaua uauguaugug |
| | uauguaugug uauauaugua uauguaugua uguaucgcgu guaugugugu |
| | auguaugcau guguauguau guauaugcau guauguguau guguauauau |
| | guaugugugu guauguauau guguguguau guguaugugu guguguaugu |
| | guguguguau guauguaugu auguauaugu auuauacaca uauacacaua |
| | uugguuuuuu uaaucauuug agaguuaguu gaagauaaaa acccaucacc |
| | ccuaaaugua uuccaaagaa uaagaacauu guuuuauaca uagcacacuu |
| | aacaaaauca agaaauuuaa cauuaauaca guacuguuac cuaauccgua |
| | gucgauuuuc aaauuuuguc aguuguucca auaauguccu uuauauauuc |
| | cccgcccagc |
| 66 | gcugggcggg gaauauauaa aggacauuau uggaacaacu gacaaaauuu |
| | gaaaaucgac uacggauuag guaacaguac uguauuaaug uuaaauuucu |
| | ugauuuuguu aagugugcua uguauaaaac aauguucuua uucuuuggaa |
| | uacauuuagg ggugaugggu uuuuaucuuc aacuaacucu caaaugauua |
| | aaaaaaccaa uauguguaua uguguauaau acauauacau acauacauac |
| | auacacacac acauacacac acacauacac auacacacac auauacauac |
| | acacacauac auauauacac auacacauac augcauauac auacauacac |
| | augcauacau acacacauac acgcgauaca uacauacaua uacauauaua |
| | cacauacaua cacauacaua uauacacaua cacacacauu uuuaaaugcc |
| | caccaaaagu |
| 67 | aauuccagcg agaggcagag ggagcgagcg ggcggccggc uaggguggaa |
| | gagccgggcg agcagagcug cgcugcgggc guccugggaa gggagauccg |
| | gagcgaauag ggggcuucgc cucuggccca gcccucccgc uugauccccc |
| | aggccagcgg uccgcaaccc uugccgcauc cacgaaacuu ugcccauagc |
| | agcgggcggg cacuuugcac uggaacuuac aacacccgag caaggacgcg |
| | acucucccga cgcggggagg cuauucugcc cauuugggga cacuuccccg |
| | ccgcugccag gacccgcuuc ucugaaaggc ucuccuugca gcugcuuaga |
| | cgcuggauuu uuuucgggua guggaaaacc agcagccucc cgcga |
| 68 | ucgcgggagg cugcugguuu uccacuaccc gaaaaaaauc cagcgucuaa |
| | gcagcugcaa ggagagccuu ucagagaagc ggguccuggc agcggcgggg |
| | aagugucccc aaaugggcag aauagccucc ccgcgucggg agagucgcgu |
| | ccuugcucgg guguuguaag uuccagugca aagugcccgc ccgcugcuau |
| | gggcaaaguu ucguggaugc ggcaaggguu gcggaccgcu ggccuggggg |
| | aucaagcggg agggcugggc cagaggcgaa gcccccuauu cgcuccggau |
| | cucccuuccc aggacgcccg cagcgcagcu cugcucgccc ggcucuucca |
| | cccuagccgg ccgcccgcuc gcucccucug ccucucgcug gaauu |
| 69 | gggcacuuug cacuggaacu uacaacaccc gagcaaggac gcgacucu |
| 70 | agagucgcgu ccuugcucgg guguuguaag uuccagugca aagugccc |
| 71 | guacugacau cquagaugga aaucauaaac ugacucuugg uuugauuugg |
| | aauauaaucc uccacuggca g |
| 72 | cugccagugg aggauuauau uccaaaucaa accaagaguc aguuuaugau |
| | uuccaucuac gaugucagua c |

### DNA Molecules and Vectors

According to a further aspect of the invention, there is provided a DNA molecule encoding a functional nucleic acid molecule of the invention.

According to a further aspect of the invention, there is provided an expression vector comprising said DNA molecule.

Exemplary expression vectors are known in the art and may include, for example, plasmid vectors, viral vectors (for example adenovirus, adeno-associated virus, retrovirus or lentivirus vectors), phage vectors, cosmid vectors and the like. The choice of expression vector may be dependent upon the type of host cell to be used and the purpose of use. In particular, and without limitation, the following plasmids have been used for expression of functional nucleic acid molecule:

### Mammalian Expression Plasmids:

pCDNA3.1 (-)
pDUAL-eGFPΔ (modified from peGFP-C2)

### Viral Vectors:

pAAV (an Adeno-Associated Virus vector)
rcLV-TetOne-Puro (a 3^{rd} generation Lentivirus vector)
pLPCX-link (a 3^{rd} generation Retrovirus vector)

In one embodiment the mammalian expression plasmid is pCDNA3.1 (-).

In another embodiment the mammalian expression plasmid is pDUAL-eGFPΔ.

Plasmids of the invention may comprise any one of more features selected from the list comprising: a CMV promoter, a H1 promoter, and/or a BGH poly(A) terminator.

In one embodiment the viral vector is pAAV.

In one embodiment the viral vector is rcLV-TetOne-Puro.

In one embodiment the viral vector is pLPCX-link.

Vectors of the invention may comprise any one of more features selected from the list comprising: a CAG promoter, a CMV enhancer, SV40 late poly(A) terminator, a LTR-TREt (Tre-Tight) promoter, and/or a BGH poly(A) terminator.

It should be noted that any promoter may be used in the vector. Since the activity of the functional nucleic acids of the invention is independent of the promoter it is envisaged that these will work just as well as those exemplified above.

### Compositions and Methods

The present invention also relates to compositions comprising the functional nucleic acid molecule, the DNA molecule or the expression vector according to the invention.

The composition may comprise components which enable delivery of said functional nucleic acid molecule by viral vectors (AAV, lentivirus and the like) and non-viral vectors (nanoparticles, lipid particles and the like). Alternatively, the functional nucleic acid molecule of the invention may be administered as naked or unpackaged RNA.

The composition may comprise components that are known in the art to aid the stability of the nucleic acid molecule, e.g., salts (such as those providing Mg²⁺ ions).

The functional nucleic acid molecule may be administered as part of a composition, for example a composition comprising a suitable carrier. In certain embodiments, the carrier is selected based upon its ability to facilitate the transfection of a target cell with one or more functional nucleic acid molecule.

Therefore, according to a further aspect of the invention, there is provided a composition comprising the functional nucleic acid molecule described herein.

In one embodiment, there is provided a pharmaceutical composition comprising at least one functional nucleic acid molecule, at least one DNA molecule, or at least one expression vector according to the present invention.

Suitably, a pharmaceutical composition may comprise at least one functional nucleic acid molecule, at least one DNA molecule, or at least one expression vector according to the present invention with a suitable pharmaceutical excipient, diluent or carrier.

The suitable pharmaceutical excipient, diluent or carrier may depend on the intended route of administration and standard pharmaceutical practice.

A suitable carrier may include any of the standard pharmaceutical carriers, vehicles, diluents or excipients known in the art and which are generally intended for use in facilitating the delivery of nucleic acids, such as RNA. Liposomes, exosomes, lipidic particles or nanoparticles are examples of suitable carriers that may be used for the delivery of RNA. In a preferred embodiment, the carrier or vehicle delivers its contents to the target cell such that the functional nucleic acid molecule is delivered to the appropriate subcellular compartment, such as the cytoplasm.

### Methods, Methods of Treatment and Medical Uses

In one aspect of the present invention, there is provided a method for enhancing translation of a target mRNA, such as a therapeutic target mRNA, in a cell comprising administering the functional nucleic acid molecule, DNA molecule, expression vector or composition as defined herein to the cell. Preferably, the cell is a mammalian cell, such as a human or a mouse cell.

According to a further aspect of the invention, there is provided an in vitro method for increasing the synthesis of a target protein in a cell or cell-free system comprising administering the functional nucleic acid molecule, DNA molecule, expression vector or the composition described herein, to the cell or cell-free system.

According to a further aspect of the invention, there is provided an in vivo method for increasing the synthesis of a target protein in a cell comprising administering the functional nucleic acid molecule, DNA molecule, expression vector or the composition described herein, to the cell or cell-free system.

According to a further aspect of the invention, there is provided a method for increasing the synthesis of a target protein in a cell comprising administering the functional nucleic acid molecule, DNA molecule, expression vector or the composition described herein, to the cell.

Preferably, the cell is a mammalian cell, such as a human or a mouse cell.

According to a further aspect of the invention, there is provided a method for increasing the protein synthesis efficiency of a target in a cell comprising administering the functional nucleic acid molecule, DNA molecule, expression vector or the composition described herein, to the cell. Preferably, the cell is a mammalian cell, such as a human or a mouse cell.

Methods of the invention result in increased levels of target protein in a cell and therefore find use, for example, in methods of treatment of diseases which are associated with gene defects (e.g. one or more gene defects which result in reduced protein levels and/or loss-of-function mutations of the encoding gene). Methods of the invention find particular use in diseases caused by a quantitative decrease in the predetermined, normal protein level, such as haploinsufficiency.

Methods of the invention can be performed in vitro, ex vivo or in vivo.

The methods described herein may comprise transfecting into a cell the functional nucleic acid molecule, DNA molecule, expression vector or composition as defined herein. The functional nucleic acid molecule, DNA molecule, expression vector or composition may be administered to target cells using methods known in the art and include, for example, microinjection, lipofection, electroporation, using calcium phosphate, self-infection by the vector or transduction of a virus.

According to a further aspect of the invention, there is provided the functional nucleic acid molecule, DNA molecule, expression vector or the composition, such as pharmaceutical composition, as defined herein for use in therapy.

According to a further aspect of the invention, there is provided the functional nucleic acid molecule, DNA molecule, expression vector or the composition, such as pharmaceutical composition, as defined herein for use as a medicament.

It will be understood that the functional nucleic acid molecule of the invention finds use in increasing the level of a target protein, such as a therapeutic target within a cell.

Thus the functional nucleic acid molecule, DNA molecule, expression vector or composition, such as a pharmaceutical composition may be administered to a subject having an existing disease or condition in order to lessen, reduce or improve at least one symptom associated with the disease and/or to slow down, reduce or block the progression of the disease.

In one aspect there is provided the functional nucleic acid molecule, DNA molecule, expression vector or composition, such as pharmaceutical composition, for use in the treatment of a disease-associated with one or more gene defects.

As used herein, “gene defect” or “gene defects”, refer to one or more abnormalities in a gene which results in reduced protein levels and/or loss-of-function mutations of the encoding gene. For example, a gene defect may be caused by a mutation in a single gene, mutations in multiple genes, chromosomal abnormality, or mutation(s) in mitochondrial DNA or in nuclear genes.

For example, a disease associated with one or more gene defects may be a cancer or a neurodegenerative disease.

In one aspect there is provided the functional nucleic acid molecule, DNA molecule, expression vector or composition, such as pharmaceutical composition, for use in the treatment of cancer.

In one aspect there is provided the functional nucleic acid molecule, DNA molecule, expression vector or composition, such as pharmaceutical composition, for use in the treatment of a neurodegenerative disease.

In one embodiment, the gene defect is a microdeletion.

In another embodiment, the microdeletion is a microdeletion of part of chromosome 22.

In another embodiment, the microdeletion of part of chromosome 22 is 22q11.2DS.

In one embodiment, there is provided the functional nucleic acid molecule, DNA molecule, expression vector or composition, such as pharmaceutical composition, for use in the treatment of a disease-associated with one or more gene defects, wherein the gene defect is a microdeletion.

In one embodiment, there is provided the functional nucleic acid molecule, DNA molecule, expression vector or composition, such as pharmaceutical composition, for use in the treatment of a disease-associated with a microdeletion, wherein the microdeletion is a microdeletion of part of chromosome 22.

In one embodiment, there is provided the functional nucleic acid molecule, DNA molecule, expression vector or composition, such as pharmaceutical composition, for use in the treatment of a disease-associated with a microdeletion of part of chromosome 22, wherein the microdeletion is 22q11.2DS.

In one aspect, there is provided a method of treating a disease associated with one or more gene defects comprising administering a therapeutically effective amount of the functional nucleic acid molecule, the DNA molecule, the expression vector, or the composition, such as pharmaceutical composition, as defined herein to a subject.

In one embodiment, there is provided a method of treating a disease associated with one or more gene defects comprising administering a therapeutically effective amount of the functional nucleic acid molecule, the DNA molecule, the expression vector, or the composition, such as pharmaceutical composition, as defined herein to a subject, wherein the gene defect is a microdeletion.

In one embodiment, there is provided a method of treating a disease associated a microdeletion comprising administering a therapeutically effective amount of the functional nucleic acid molecule, the DNA molecule, the expression vector, or the composition, such as pharmaceutical composition, as defined herein to a subject, wherein the microdeletion is a microdeletion of part of chromosome 22.

In one embodiment, there is provided a method of treating a disease associated a microdeletion of part of chromosome 22 comprising administering a therapeutically effective amount of the functional nucleic acid molecule, the DNA molecule, the expression vector, or the composition, such as pharmaceutical composition, as defined herein to a subject, wherein the microdeletion is a microdeletion is 22q11.2DS.

In one aspect, there is provided a method of treating a disease associated with one or more gene defects comprising administering a therapeutically effective amount of the functional nucleic acid molecule, the DNA molecule, the expression vector, or the composition, such as the pharmaceutical composition, as defined herein to a subject in need thereof, wherein the disease is a cancer or a neurodegenerative disease.

Herein instances of the plural form of words should be taken to cover also the singular form of the word and vice versa, unless the context clearly dictates otherwise.

The invention will now be illustrated with reference to the following non-limiting examples.

### EXAMPLES

### Example 1—Target Determination by Mono-BD Screening

Nine genes were chosen for initial screening of mono-BD-SINEUP (i.e., a SINEUP with one BD) as proof of concept that the genes would be susceptible to SINEUP-mediated transcriptional upregulation. The targets selected were: TBX-1, HIRA, DGCR8, COMT, PRODH, SEPT5, ZDHHC8, RANBP1, RTN4R.

The expression levels and the isoforms of each target gene were analyzed. Specific primers were used to evaluate the presence of the target transcripts in mouse bran (cortex, hippocampus and striatum) and in cell lines used for the SINEUP screening (neuro2A or astrocytes). All targets were expressed in mouse brain, most targets were also present in the neuro2A cell line except COMT, PRODH and RTN4R, which were expressed only in astrocytes (data not shown). Based on the identity of the specific isoform present for each mRNAtarget, 3 to 4 miniSINEUPs for each target were designed and synthetized. All SINEUP were transfected in 6-well plate, 1 μg of plasmid for each well. 48 hours post-transfection, half of the cells were used for protein extraction and WB analysis, and half of the cells for RNA extraction and qPCR analysis.

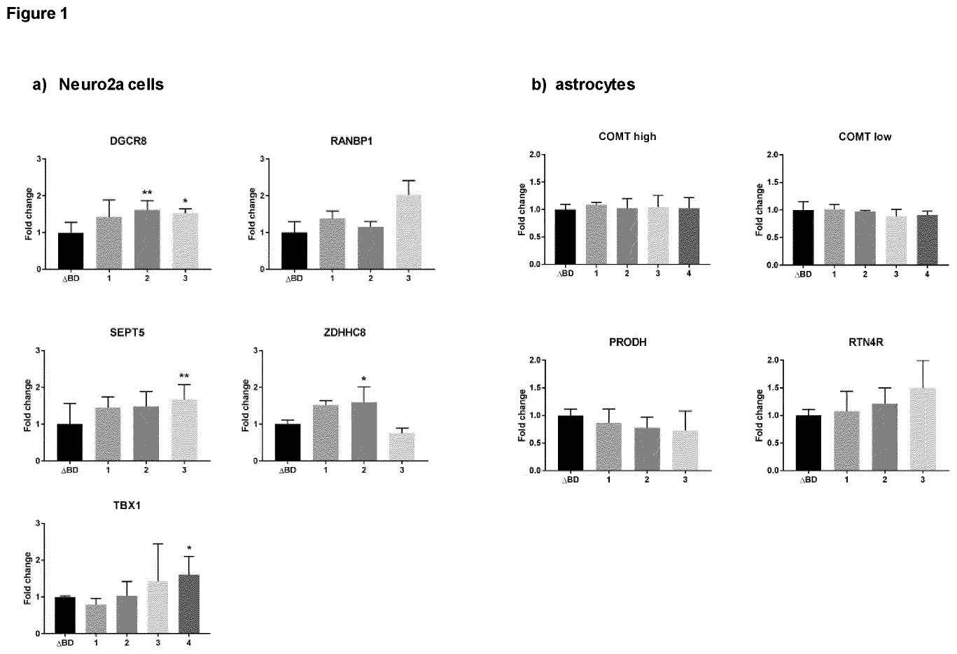

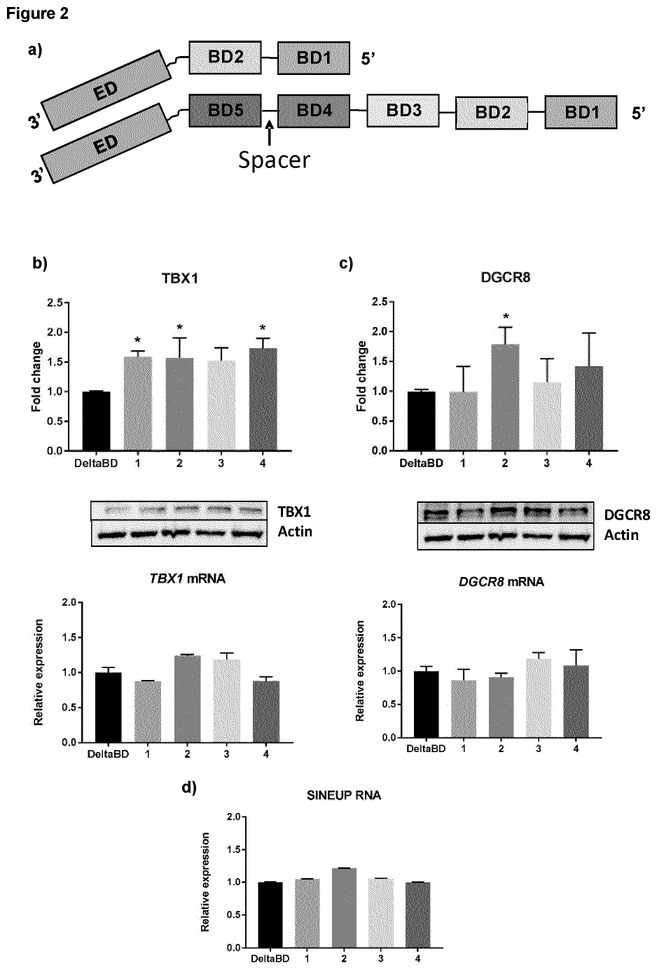

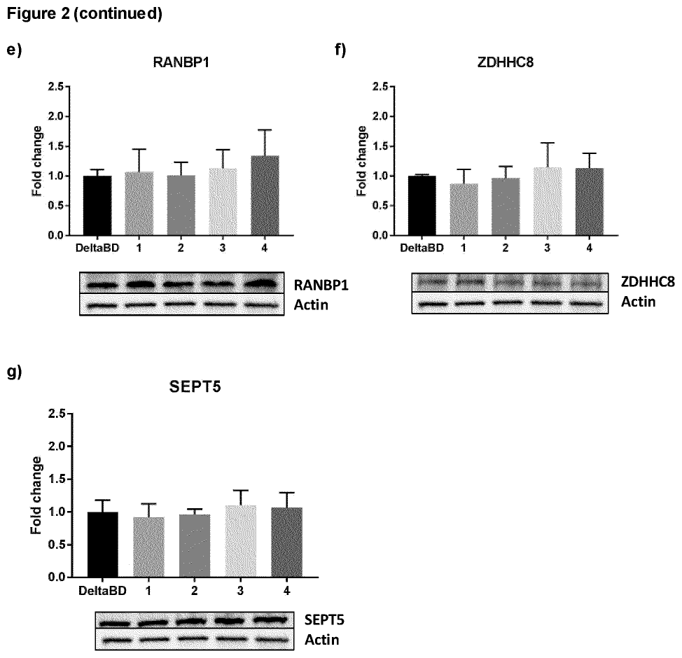

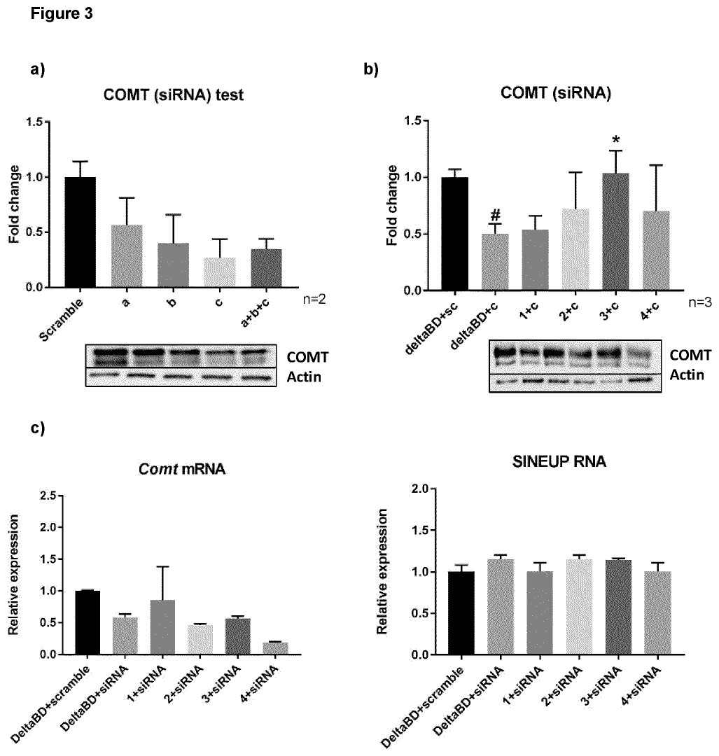

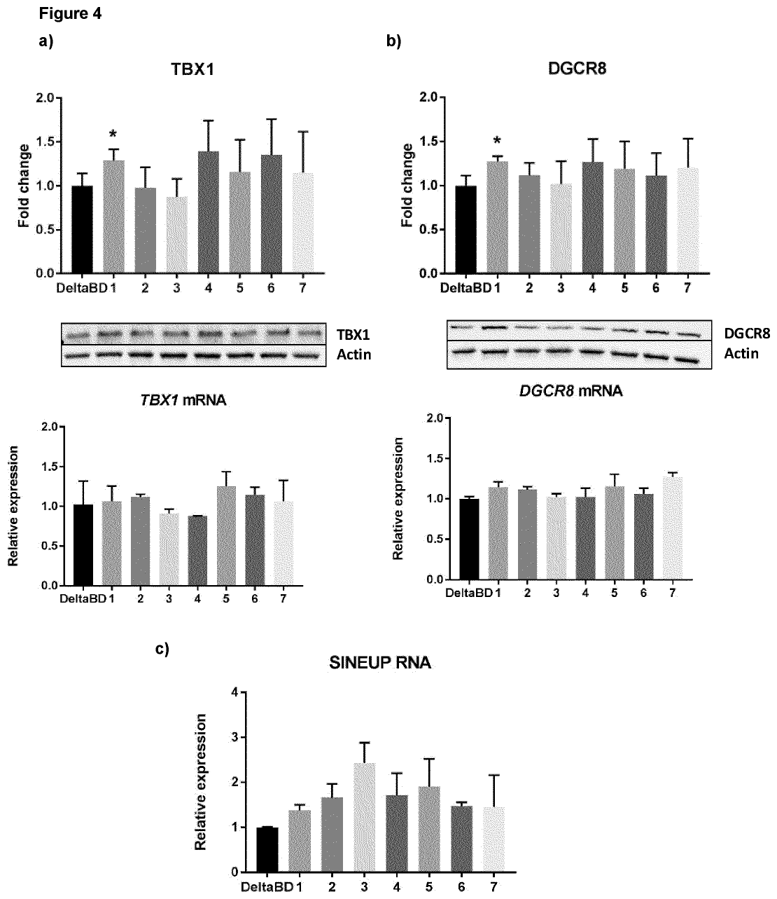

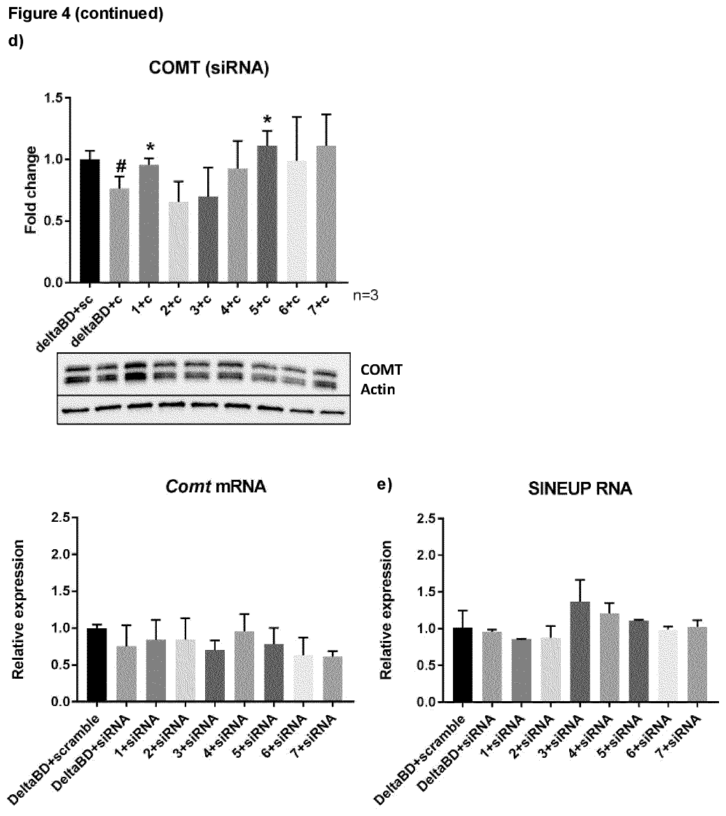

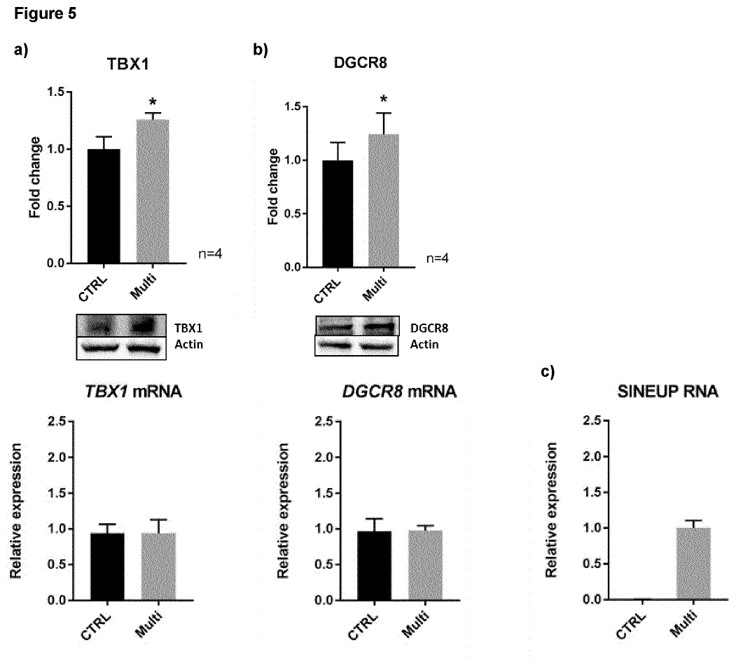

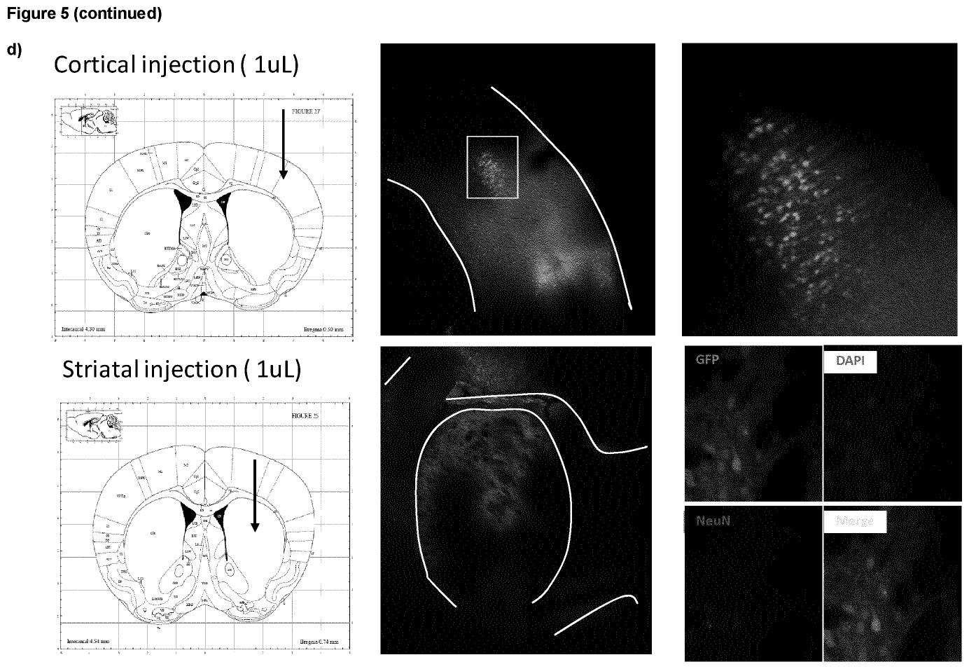

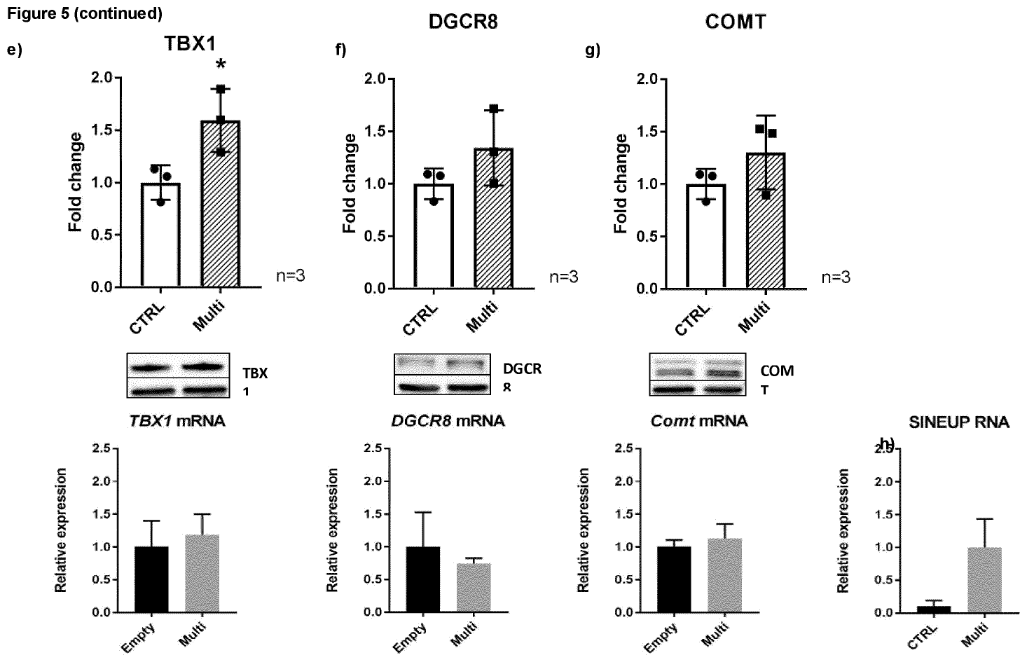

From this first in vitro screening, it was found that some miniSINEUPs were able to increase protein expression from the target m RNA in neuro2A cells. However, none of the tested miniSINEUPs were able to increase protein expression from targets expressed in astrocytes (FIG. 1).

### Example 2—Multi-BD Screening

Following proof of principle experiments, utilizing mono-BDs (Example 1), a series of multi-BD-SINEUPs were designed with the following BD:
1=TBX1-DGCR8 (2-BD-SINEUP)
2=DGCR8-TBX1 (2-BD-SINEUP)
3=TBX1-RANBP1-SEPT5-ZDHHC8-DGCR8 (5-BD-SINEUP)
4=DGCR8-RANBP1-SEPT5-ZDHHC8-TBX1 (5-BD-SINEUP)

In all cases BDs were separated by a spacer of 19 nucleotides in length and the order of BDs is 5′ to 3′ as read from left to right, e.g., in ‘1’ the order is 5′-TBX1-DGCR8-3′.

These multi-BD SINEUPs were tested in vitro in neuro2A cells (FIG. 2). All SINEUP were transfected in 6-well plate, 1 μg of plasmid for each well. 48 hours post-transfection, half of the cells were used for protein extraction and WB analysis, and half of the cells for RNA extraction and qPCR analysis. While the 2-BD-SINEUPs exhibited some activity, the 5-BD-SINEUPS did not increase target protein levels.

### Example 3—siRNA-Induced Downregulation of COMT mRNA

In order to assess the apparent inability of the tested SINEUPs to upregulate COMT expression, siRNA was employed to downregulate COMT expression, in order to mimic a haploinsufficient phenotype in astrocytes. The four SINEUPs of Example 2 (1-4) were tested for their ability to promote protein expression of COMT following siRNA treatment.

Three different siRNAs were obtained from Origene and tested for their ability to reduce COMT protein expression levels in astrocytes (FIG. 3a). The single most effective siRNA, ‘c’, was rested in combination with the 4 mono-BD SINEUPs previously utilized in Example 2 (i.e., 1-4). Putatively functional BDs were compared to ΔBD-SINEUP (no binding domains) in conjunction with a putatively inactive scrambled siRNA. All SINEUP were transfected in 6-well plate, 1 μg of plasmid for each well in co-transfection with the siRNA. 48 hours post-transfection, half of the cells were used for protein extraction and WB analysis, and half of the cells for RNA extraction and qPCR analysis.

One mono-BD SINEUP (‘3’) induced a significant increase in COMT protein expression, in the presence of siRNA ‘c’ (FIG. 3), without increasing COMT mRNA levels relative to the ΔBD-SINEUP+siRNA control.

### Example 4—siRNA-Induced Downregulation of COMT mRNA

A series of new multi-BD SINEUPs were designed and synthetized based on the results of Example 3, in order to include an effective BD for COMT. The new multi-BD series was as follows:
1=DGCR8-TBX1-COMT (3-BD-SINEUP)
2=DGCR8-TBX1-COMT-RANBP1 (4-BD-SINEUP)
3=DGCR8-TBX1-COMT-ZDHHC8 (4-BD-SINEUP)
4=DGCR8-TBX1-COMT-SEPT5 (4-BD-SINEUP)
5=DGCR8-TBX1-RANBP1-COMT (4-BD-SINEUP)
6=DGCR8-TBX1-ZDHHC8-COMT (4-BD-SINEUP)
7=DGCR8-TBX1-SEPT5-COMT (4-BD-SINEUP)

All the BD were separated by a spacer of 19 nucleotides in length.

These newly designed and synthesized multi-BD-SINEUPs were tested in both neuro2A cells and astrocytes, which had been transfected with the siRNA (‘c’) directed toward the COMT transcript prior to SINEUP treatment. All SINEUP were transfected in 6-well plate, 1 μg of plasmid for each well. 48 hours post-transfection, half of the cells were used for protein extraction and WB analysis, and half of the cells for RNA extraction and qPCR analysis.

No protein expression increasing activity was observed for RANBP1, ZDHHC8, or SEPT5, when treated with any of SINEUPs 1-7 of this Example.

The 3-BD-SINEUP ‘1’ increased protein expression of all three targets thereof (TBX1, DGCR8, and COMT), while the other multi-BD-SINEUPs displayed little or no activity (FIG. 4).

### Example 5—In Vitro and In Vivo Testing of an AAV Expressed Multi-BD SINEUP Targeting TBX1, DGCR8 and COMT

In order to test the most promising candidate multi-BD-SINEUP in vivo, the 3-BD-SINEUP (‘1’) (hereafter ‘the 3-BD-SINEUP’) was cloned into a pAAV vector that express the SINEUP under control of the CAG promoter and GFP reporter under the PGK promoter.

In neuro2A cells, In vitro, the pAAV plasmid was as efficient at increasing protein expression as the previous tests (FIG. 5a-c).

An AAV1/2 with a titer of 5×10¹¹ vector genomes/ml was produced from a transfection of HEK-293T cells. Subsequently, 1 μL of AAV expressing the 3-BD-SINEUP was injected in the somatosensory cortex or the dorsal striatum of mice to evaluate the expression and the diffusion of the AAV.

Five weeks after injection, the mice were perfused with 4% PFA and then the brains were dissected and 35 μm slices were prepared for immunohistochemistry analysis. Confocal microscopy revealed that the AAV-3-BD-SINEUP was efficiently expressed in and infected neurons of the two brain areas tested (FIG. 5d).

To measure the efficacy of the 3-BD-SINEUPs in vivo, 3 mice were injected with the AAV-CTRL in the left somatosensory cortex and with AAV-3-BD-SINEUP in the right somatosensory cortex. Five weeks after injection, mice were sacrificed and brain were dissected. The dissected brain region was broken apart on dry ice to make a homogeneous powder. Half of this mix was used to extract proteins for WB analysis and a half for RNA extraction and qPCR analysis. 3-BD-SINEUP was able to increase TBX-1 expression in all mice and to increase COMT and DGCR8 expression in 2 out of 3 mice (FIG. 5e-h).

SEQUENCES

| SEQ ID | |
|---|---|
| NO: | Exemplary spacer sequence |
| 1 | aucugcagaa uuc |
| SEQ ID | |
| NO: | Exemplary SINE B2 sequence |
| 2 | cagugcuaga ggaggucaga agagggcauu ggauccccca gaacuggagu |
| | uauacgguaa ccucguggug guugugaacc accaugugga uggauauuga |
| | guuccaaaca cugguccugu gcaagagcau ccagugcucu uaagugcuga |
| | gccaucucuu uagcucc |
| 3 | gaacuggagu uauacgguaa ccucguggug guugugaacc accaugugga |
| | uggauauuga guuccaaaca cuggucc |
| 4 | ccucguggug guugugaacc accaugugg |
| 5 | guuauacggu aaccucgugg ugguugugaa ccaccaugug gauggauauu |
| | gaguuccaaa |
| 6 | aucccccaga acuggaguua uacgguaacc ucgugguggu ugugaaccac |
| | cauguggaug gauauugagu uccaaacacu gguccugugc aagagcau |
| 7 | gaagagggca uuggaucccc cagaacugga guuauacggu aaccucgugg |
| | ugguugugaa ccaccaugug gauggauauu gaguuccaaa cacugguccu |
| | gugcaagagc auccagugcu cuuaagugc |
| 8 | gggcagugcu agaggagguc agaagagggc auuggauccc ccagaacugg |
| | aguuauacgg uaaccucgug gugguuguga accaccaugu ggauggauau |
| | ugaguuccaa acacuggucc ugugcaagag cauccagugc ucuuaagugc |
| | ugagccaucu cuuuagcucc agucucuuaa gcu |
| 9 | gaacuggagu uauacgguaa ccucguggug guugugaacc accaugugga |
| | uggauauuga guuccaa |
| 10 | ggauccccca gaacuggagu uauacgguaa ccucguggug guugugaacc |
| | accaugugga uggauauuga guuccaaaca cugguccugu gcaagagcau |
| | ccagugc |
| 11 | agagggcauu ggauccccca gaacuggagu uauacgguaa ccucguggug |
| | guugugaacc accaugugga uggauauuga guuccaaaca cugguccugu |
| | gcaagagcau ccagugcucu uaagugc |
| 12 | gguaaccucg uggugguugu gaaccaccau guggaugg |
| 13 | cagugcuaga ggaggucaga agagggcauu ggauccccca gaacuggagu |
| | uauacgauaa ccucguggug guugugaacc accaugugga uggauauuga |
| | guuccaaaca cugguccugu gcaagagcau ccagugcucu uaagugcuga |
| | gccaucucuu uagcuccagu cucuuaagcu |
| 14 | cagugcuaga ggaggucaga agagggcauu ggauccccca gaacuggagu |
| | uauacgcuaa ccucguggug guugugaacc accaugugga uggauauuga |
| | guuccaaaca cugguccugu gcaagagcau ccagugcucu uaagugcuga |
| | gccaucucuu uagcuccagu cucuuaagcu |
| 15 | cagugcuaga ggaggucaga agagggcauu ggauccccca gaacuggcgu |
| | uauacgguaa ccucguggug guugugaacc accaugugga uggauauuga |
| | guuccaaaca cugguccugu gcaagagcau ccagugcucu uaagugcuga |
| | gccaucucuu uagcuccagu cucuuaagcu |
| 16 | cagugcuaga ggaggucaga agagggcauu ggauccccca gaaguggagu |
| | uauacgguaa ccucguggug guugugaacc accaugugga uggauauuga |
| | guuccaaaca cugcuccugu gcaagagcau ccagugcucu uaagugcuga |
| | gccaucucuu uagcuccagu cucuuaagcu |
| 17 | cagugcuaga ggaggucaga agagggcauu ggauccccca gauggugagu |
| | uauacgguaa ccucguggug guugugaacc accaugugga uggauauuga |
| | guuccaaaca cgucaccugu gcaagagcau ccagugcucu uaagugcuga |
| | gccaucucuu uagcuccagu cucuuaagcu |
| 18 | cagugcuaga ggaggucaga agagggcauu ggauccccca gaacugcacu |
| | auacgguaac cucguggugg uugugaacca ccauguggau ggauauugag |
| | uuccaaauga gugguccugu gcaagagcau ccagugcucu uaagugcuga |
| | gccaucucuu uagcuccagu cucuuaagcu |
| 19 | gggcauugga ucccccagaa cuggaguuau acgguaaccu cguggugguu |
| | gugaaccacc auguggaugg auauugaguu ccaaacacug guccugugca |
| | agagcaucca gugcuc |
| 20 | ggacuggagu uauacgguaa ccucguggug guugugaacc accaugugga |
| | uggauauuga guuccaaaca cuggucc |
| 21 | gaacuggcgu uauacgguaa ccucguggug guugugaacc accaugugga |
| | uggauauuga guuccaaaca cuggucc |
| 22 | gaugccuuag aaguggaguu aagaguugug agcugccguu uuuugguucu |
| | gggacucgaa cucguuuccu cugauacuau caaccaccaa gccaucucuu |
| | cagcccc |
| 23 | gccagaagaa guugugggau ucccuggaac uggagcaacc aacaguuugu |
| | gugcaccaug uggguaaugg gaaucgaacc uggguccucu auaagacugg |
| | ccagugcucu uaacuacuga ggugcauuuc u |
| 24 | uuauuuuaaa uauaugagua uuucaccugc auaggcgcac aguacccaca |
| | gagacuagaa gaggguggca gaucuccuga gacuggaguu aaugcuugug |
| | agcugccaug uggaugcugg aaaucaaacc cagguccuuu ggaaggcagg |
| | caggugcucu uaaucaugga agcaucucuu cagcucc |
| 25 | cagcgacauc agaagaggau auuggauccc auuacagaug guugaaggcc |
| | accaugucgu ugcugggaau gaacucaaga ccucuggaag agcagucagu |
| | gcucuuaacc ucugagccau cucuccagcc c |
| 26 | auccccucca aagcucaaga ugguuguaag ccacccugug auugcuggga |
| | uuugaacuca agaccuccgg aagagcaauu agugcucuua accgcugagc |
| | aaucucucca gccc |
| 27 | gugcagugcu agaggagguc agaagagggc auuggauccc ccagaacugg |
| | aguuauacgg uaaccucgug gugguuguga accaccaugu ggauggauau |
| | ugaguuccaa acacuggucc ugugcaagag cauccagugc ucuuaagugc |
| | ugagccaucu cuuuagcucc uuauuuuaaa uauaugagua uuucaccugc |
| | auaggcgcac aguacccaca gagacuagaa gaggguggca gaucuccuga |
| | gacuggaguu aaugcuugug agcugccaug uggaugcugg aaaucaaacc |
| | cagguccuuu ggaaggcagg caggugcucu uaaucaugga agcaucucuu |
| | cagcucc |
| 28 | gugcagugcu agaggagguc agaagagggc auuggauccc ccagaacugg |
| | aguuauacgg uaaccucgug gugguuguga accaccaugu ggauggauau |
| | ugaguuccaa acacuggucc ugugcaagag cauccagugc ucuuaagugc |
| | ugagccaucu cuuuagcucc gugcgaauuc ggugcagugc uagaggaggu |
| | cagaagaggg cauuggaucc cccagaacug gaguuauacg guaaccucgu |
| | ggugguugug aaccaccaug uggauggaua uugaguucca aacacugguc |
| | cugugcaaga gcauccagug cucuuaagug cugagccauc ucuuuagcuc |
| | cgugcgaauu cggugcagug cuagaggagg ucagaagagg gcauuggauc |
| | ccccagaacu ggaguuauac gguaaccucg uggugguugu gaaccaccau |
| | guggauggau auugaguucc aaacacuggu ccugugcaag agcauccagu |
| | gcucuuaagu gcugagccau cucuuuagcu cc |
| 29 | uuuuuuuaaa aauuuauuuu uauuuuaugu guaugagugu uuugccugca |
| | uguaugucug uguaccacgu gcgugccugg ugcccgcgga ggccagaaga |
| | gggcgucgga uccccuggaa cuggaguuac agaugguugu gagccgccau |
| | gugggugcug ggaaucgaac ccggguccuc uggaagagca gccagugcuc |
| | uuaaccgcug agccaucucu ccagcccc |
| 30 | uuuuuuuuac uuguauaggu guuuugccug cauguguauc uaucuaugua |
| | ccgaauaugu uccugguauc cacagagacc aaaaguggau guuguaucuc |
| | cugaaauugg agucauagac aguuaugagc ugccauuuga gugcuuggaa |
| | uagaacccag guccucuuaa agagcaucca gugcucuuaa aaacugagac |
| | aucucuguag ccuc |
| 31 | uuuauuuugc uuuauguguc ugaguguuug cuugaaugua ugucugugua |
| | ccacgccugu accuugugcc uucagaguug agaggagggc auaggaucuc |
| | cuggaacugg aauugcaggu gguugugagc cacccugugg guccugggga |
| | ccauacucca gcaagaacau caugugcucu uaauuccuga gucuccaacc |
| 32 | uuuauuuacu uaucuuuaug uguaugagug uguugucaga cuguuauguc |
| | ugugugucac augcaugccu gcuguucaug gaguccagaa gagggcaucg |
| | gauccccugg aacuggaguu acagaugagu ggccauguga auguuaagaa |
| | ccaaaccugg guccucugaa agagcagaca augcucuuaa cuacugagcu |
| | gucucuccag cccc |
| 33 | uuauuuuauu cguguaagug uuuugccagc aucuaugucu ucgcacuaug |
| | ugcaggucug gugccugagg gguccagacg agagcacugg gucuccggga |
| | acuggaguua cagaucauug ugagccacca ugugggugca gggaaucgaa |
| | ccugggaccu cuggaggage agccacugcu cuuaaccacu acacuauuuc |
| | uccag |
| 34 | ucuguggacc acuguguaca gaagccugag aaggcuagca gauccccaga |
| | acuggaacug ugagacgcug ugcuauggag gugcuaggaa cugaaaaugg |
| | auggguccuc ugcaagagca g |
| 35 | uuguuuuaau ugaauggcua uaggguguuu cuucuguaug uauaucuaug |
| | uuugguaccu acagaggcau cagauccucu ggaacuguag uugcugacag |
| | uugugagcug ucauggggau gcuggaauug aaccuggauc cuaugaaaga |
| | acagccagug uucuuaaccg cugagcuauc ucuccaggcc c |
| 36 | uuuuuuuuuu aauuuuaaaa aaaaagauuu uauuuauuua uuuuauauau |
| | gaugaguaca cugucacucu uuucagacac ccuagaaaag gggggcauca |
| | gaucccauua cagaugguug ugagccacau gguugcuggg aauugaccuc |
| | aggaccucug aaagagcagu cagugcucuc aaccuuugag ucaucucucc |
| | agccc |
| 37 | auguauaucu guaaugggac auacucacau acaugggcac gugaguauaa |
| | aaggccagaa gagagcacug gacccucugg aguugagauu cuaagcaguu |
| | gugaaccauc ugauguaggu gcugggaacu gaacuugggu ccuuugcuag |
| | agaaguaugu cucuuaacca cugagccgua ucuccauccc |
| 38 | uaaagauuua uucauuaagu acacuguage uaucuucaga cgcaucagaa |
| | gagggcguca gaucucuuua caggugguug ugagccacca ugugguugcu |
| | ggaauuugaa cucaggaccu ucaaaagagc agucaguguu cuuaaccgcu |
| | gagccaucuc uccaacccc |
| 39 | uuauuuauua uaaguacacu guagcugucu ucagacacaa caaaagaggg |
| | cgucagaucu cauuacaggu gguugagcca ccaugugguu gcugggauuu |
| | gaacucagga ccuucagaac agucagugcu cuuacccacu gagccagcga |
| | gccagcccc |
| 40 | gaacuggagu uauacgguaa ccucguggug guugggaacc accaugugga |
| | uggauauuga guuccaaaca cuggucc |
| 41 | gaacuggagu uauacgguaa ccucguggug guucccaacc accaugugga |
| | uggauauuga guuccaaaca cuggucc |
| 42 | ggaccggagu uauacgguaa ccgcguggug guugugaacc accacgcgga |
| | uggauauuga guuccaaaca ccggucc |
| 43 | gaacuagagu uauacgguaa ccacauggug guugugaacc accaugugga |
| | uggauauuga guuccaaaca cuaguuc |
| 44 | uuauuuuaaa uauaugagua uuucaccugc auaggcgcac aguacccaca |
| | gagacuagaa gaggguagua gauccccuag aacuggaguu auacgguaac |
| | cucguggugg uugugagcua ccauguggau ggauacuggg aaucaaaccc |
| | agguccugug gaaggcaggc aggugcucuc aagcacugag ccaucucuuc |
| | agcucc |
| 45 | uuauuuuaaa uauaugagua uuucaccugc auaggcgcac agugcucaag |
| | gagaucagaa gagggcauca gaucuccuga gacuggaguu auacgguaac |
| | cucgugaugg uugugaacua ccauguggau ggauauugag uuccaaacac |
| | agguccugug caagagcage aggugcucuu aagcacggaa ccaucucuuu |
| | agcucc |
| 46 | gaggcuagaa gaggguauca gauccccuga gacuggaguu auacgguaac |
| | cucguggugg uugugagcca ccauguggau ggauacugag aaccaaaccc |
| | ugguccugug caagagcauc aggugcucuu aagcacggaa ccaucucuuc |
| | agcucc |
| 47 | guccugugca agagcaucga acucggugcu cuuaagcaca gaagccacca |
| | agccaucucu ucagcccc |
| 48 | cagugcuaga ggaggucaga agagggcauc ccccagccuc guggugguug |
| | ugaaccacca uguggcugug caagagcaug cucuuaagug cugagccauc |
| | ucuuuagcuc |
| 49 | gagggcauug gaucccccag aacuggaguu auacgguaac cucguggugg |
| | uugugaacca ccauguggau ggauauugag uuccaaacac ugguccugug |
| | caagagcauc cagugcucuu aagugc |
| 50 | ggauccccca gaacuggagu uauacgguaa ccucguggug guugugaacc |
| | accaugugga uggauauuga guuccaaaca cugguccug |
| 51 | ugcuagagga ggucagaaga gggcauugga ugcaaaucca gugcucuuaa |
| | gugcugagcc aucucuuuag cu |
| 52 | gagggcauug gaucccccag aacuggaguu auacgguaac gauggauauu |
| | gaguuccaaa cacugguccu gugcaagagc auccagugcu cuua |
| 53 | CAGTGCTAGAGGAGGTCAGAAGAGGGCATTGGATCCCCCAGAACTGGAGTTATACGGTA |
| | ACCTCGTGGTGGTTGTGAACCACCATGTGGATGGATATTGAGTTCCAAACACTGGTCCT |
| | GTGCAAGAGCATCCAGTGCTCTTAAGTGCTGAGCCATCTCTTTAGCTCCAGTCTCTTAA |
| | AAAACAAACAAACGAACGAACAGCAAGGGAGCTGGGTATGACAACACATACTATAATTC |
| | TAGTACTCAGGATGCTGAAACAGGAGGATTGCCTGACTGGGAGATATAAGGAGAATCTG |
| | TTGTCACCCCCACCCCTCCCCATAAAGGCAGAATAAAAGAACGTCCTATAAACAAATAA |
| | ACAAACAACCCAATAAAACAAAACCAAGATCTCTCCACCTTTTCTTTGCTTTTTCAGAC |
| | TTTGTAATAAGGCCCTTTGGAGTGCAGGATATTCGGCAGGACAAGCAGAGAGGGAGACC |
| | ATCAGTTCTTTCTTTGATCAAGAAGACTATGTTCCTTAGCAAACTGGTGTGTATTATCT |
| | CTTATGCAATGAGCCTGGAAAGAGGGCACAGCCACCGAGGATGGTACAGCATGGATGGA |
| | TGGTACGCTACAGAGACTCGGGAGCCCAACTGTGAGTGGCTGACTGGCATGGTAGGTTC |
| | AGGGAAGAATTGGCCTGTGAAGAAAATGTTCTTGAAAAGTGAACAAGGTGCAGGAGGTA |
| | GGAGTGGGTCCTGGGCAAAGCAGGGGGTGCATCCCAGCCTCAGGGAATAGCACAGCAGA |
| | GGTCTGTTGATGCATGCGAGTGCATGACCTGCTTGCCAATAGACGATCAAGAATGGGCA |
| | AAGCATCATGGGTGATGAGTGGGAGAGGGGATGAGACATTCCTTTCTCCCTGCTGAGAC |
| | TTCCATTGAACCGATGAGTTCTGAATAGAAGATGCCCCCCCACCCCCCCACCAGTGTAG |
| | AATCTGAAGGGAGGCATATATTACCCTATATTACTCTGTGTTGGCGGCGAGCTATCTGA |
| | CAGCCAACCTTCCCATACATTTCATTGGGCATACACTAATGACAGGAAGTTCCTTTTGC |
| | TTGTATGCAAGAGATGGCTCACACGATGGAGAATTTAATCTTGAAGGGC |
| 54 | GATGCGGCCGCCACTGTGCTGGATATCTGCAGAATTCGCCCTTCAGTGCTAGAGGAGGT |
| | CAGAAGAGGGCATTGGATCCCCCAGAACTGGAGTTATACGGTAACCTCGTGGTGGTTGT |
| | GAACCACCATGTGGATGGATATTGAGTTCCAAACACTGGTCCTGTGCAAGAGCATCCAG |
| | TGCTCTTAAGTGCTGAGCCATCTCTTTAGCTCCAGTCTCTTAAAAAACAAACAAACGAA |
| | CGAACAGCAAGGGAGCTGGGTATGACAACACATACTATAATTCTAGTACTCAGGATGCT |
| | GAAACAGGAGGATTGCCTGACTGGGAGATATAAGGAGAATCTGTTGTCACCCCCACCCC |
| | TCCCCATAAAGGCAGAATAAAAGAACGTCCTATAAACAAATAAACAAACAACCCAATAA |
| | AACAAAACCAAGATCTCTCCACCTTTTCTTTGCTTTTTCAGACTTTGTAATAAGGCCCT |
| | TTGGAGTGCAGGATATTCGGCAGGACAAGCAGAGAGGGAGACCATCAGTTCTTTCTTTG |
| | ATCAAGAAGACTATGTTCCTTAGCAAACTGGTGTGTATTATCTCTTATGCAATGAGCCT |
| | GGAAAGAGGGCACAGCCACCGAGGATGGTACAGCATGGATGGATGGTACGCTACAGAGA |
| | CTCGGGAGCCCAACTGTGAGTGGCTGACTGGCATGGTAGGTTCAGGGAAGAATTGGCCT |
| | GTGAAGAAAATGTTCTTGAAAAGTGAACAAGGTGCAGGAGGTAGGAGTGGGTCCTGGGC |
| | AAAGCAGGGGGTGCATCCCAGCCTCAGGGAATAGCACAGCAGAGGTCTGTTGATGCATG |
| | CGAGTGCATGACCTGCTTGCCAATAGACGATCAAGAATGGGCAAAGCATCATGGGTGAT |
| | GAGTGGGAGAGGGGATGAGACATTCCTTTCTCCCTGCTGAGACTTCCATTGAACCGATG |
| | AGTTCTGAATAGAAGATGCCCCCCCACCCCCCCACCAGTGTAGAATCTGAAGGGAGGCA |
| | TATATTACCCTATATTACTCTGTGTTGGCGGCGAGCTATCTGACAGCCAACCTTCCCAT |
| | ACATTTCATTGGGCATACACTAATGACAGGAAGTTCCTTTTGCTTGTATGCAAGAGATG |
| | GCTCACACGATGGAGAATTTAATCTTGAAGGGC |
| SEQ ID | |
| NO: | Exemplary IRES Sequence |
| 55 | gccagccccc ugaugggggc gacacuccac caugaaucac uccccuguga |
| | ggaacuacug ucuucacgca gaaagcgucu agccauggcg uuaguaugag |
| | ugucgugcag ccuccaggac ccccccuccc gggagagcca uaguggucug |
| | cggaaccggu gaguacaccg gaauugccag gacgaccggg uccuuucuug |
| | gauaaacccg cucaaugccu ggagauuugg gcgugccccc gcaagacugc |
| | uagccgagua guguuggguc gcgaaaggcc uugugguacu gccugauagg |
| | gugcuugcga gugccccggg aggucucgua gaccgugcac caugagcacg |
| | aauccuaaac cucaaagaaa aaccaaacgu aac |
| 56 | guuacguuug guuuuucuuu gagguuuagg auucgugcuc auggugcacg |
| | gucuacgaga ccucccgggg cacucgcaag cacccuauca ggcaguacca |
| | caaggccuuu cgcgacccaa cacuacucgg cuagcagucu ugcgggggca |
| | cgcccaaauc uccaggcauu gagcggguuu auccaagaaa ggacccgguc |
| | guccuggcaa uuccggugua cucaccgguu ccgcagacca cuauggcucu |
| | cccgggaggg gggguccugg aggcugcacg acacucauac uaacgccaug |
| | gcuagacgcu uucugcguga agacaguagu uccucacagg ggagugauuc |
| | augguggagu gucgccccca ucagggggcu ggc |
| 57 | augagucugg acaucccuca ccggugacgg ugguccaggc ugcguuggcg |
| | gccuaccuau ggcuaacgcc augggacgcu aguugugaac aaggugugaa |
| | gagccuauug agcuacauaa gaauccuccg gccccugaau gcggcuaauc |
| | ccaaccucgg agcagguggu cacaaaccag ugauuggccu gucguaacgc |
| | gcaaguccgu ggcggaaccg acuacuuugg guguccgugu uuccuuuuau |
| | uuuauugugg cugcuuaugg ugacaaucac agauuguuau cauaaagcga |
| | auuggauugg cc |
| 58 | ggccaaucca auucgcuuua ugauaacaau cugugauugu caccauaagc |
| | agccacaaua aaauaaaagg aaacacggac acccaaagua gucgguuccg |
| | ccacggacuu gcgcguuacg acaggccaau cacugguuug ugaccaccug |
| | cuccgagguu gggauuagcc gcauucaggg gccggaggau ucuuauguag |
| | cucaauaggc ucuucacacc uuguucacaa cuagcguccc auggcguuag |
| | ccauagguag gccgccaacg cagccuggac caccgucacc ggugagggau |
| | guccagacuc au |
| 59 | cccccccucu cccucccccc ccccuaacgu uacuggccga agccgcuugg |
| | aauaaggccg gugugcguuu gucuauaugu uauuuuccac cauauugccg |
| | ucuuuuggca augugagggc ccggaaaccu ggcccugucu ucuugacgag |
| | cauuccuagg ggucuuuccc cucucgccaa aggaaugcaa ggucuguuga |
| | augucgugaa ggaagcaguu ccucuggaag cuucuugaag acaaacaacg |
| | ucuguagcga cccuuugcag gcagcggaac cccccaccug gcgacaggug |
| | ccucugcggc caaaagccac guguauaaga uacaccugca aaggcggcac |
| | aaccccagug ccacguugug aguuggauag uuguggaaag agucaaaugg |
| | cucuccucaa gcguauucaa caaggggcug aaggaugccc agaagguacc |
| | ccauuguaug ggaucugauc uggggccucg gugcacaugc uuuacaugug |
| | uuuagucgag guuaaaaaac gucuaggccc cccgaaccac ggggacgugg |
| | uuuuccuuug aaaaacacga ugauaa |
| 60 | uuaucaucgu guuuuucaaa ggaaaaccac guccccgugg uucggggggc |
| | cuagacguuu uuuaaccucg acuaaacaca uguaaagcau gugcaccgag |
| | gccccagauc agaucccaua caauggggua ccuucugggc auccuucagc |
| | cccuuguuga auacgcuuga ggagagccau uugacucuuu ccacaacuau |
| | ccaacucaca acguggcacu gggguugugc cgccuuugca gguguaucuu |
| | auacacgugg cuuuuggccg cagaggcacc ugucgccagg ugggggguuc |
| | cgcugccugc aaagggucgc uacagacguu guuugucuuc aagaagcuuc |
| | cagaggaacu gcuuccuuca cgacauucaa cagaccuugc auuccuuugg |
| | cgagagggga aagaccccua ggaaugcucg ucaagaagac agggccaggu |
| | uuccgggccc ucacauugcc aaaagacggc aauauggugg aaaauaacau |
| | auagacaaac gcacaccggc cuuauuccaa gcggcuucgg ccaguaacgu |
| | uagggggggg ggagggagag gggggg |
| 61 | aaagcaaaaa ugugaucuug cuuguaaaua caauuuugag agguuaauaa |
| | auuacaagua gugcuauuuu uguauuuagg uuagcuauuu agcuuuacgu |
| | uccaggaugc cuaguggcag ccccacaaua uccaggaagc ccucucugcg |
| | guuuuucaga uuagguaguc gaaaaaccua agaaauuuac cu |
| 62 | agguaaauuu cuuagguuuu ucgacuaccu aaucugaaaa accgcagaga |
| | gggcuuccug gauauugugg ggcugccacu aggcauccug gaacguaaag |
| | cuaaauagcu aaccuaaaua caaaaauagc acuacuugua auuuauuaac |
| | cucucaaaau uguauuuaca agcaagauca cauuuuugcu uu |
| 63 | cagagaucca ggggaggcgc cugugaggcc cggaccugcc ccggggcgaa |
| | ggguaugugg cgagacagag cccugcaccc cuaauucccg guggaaaacu |
| | ccuguugccg uuucccucca ccggccugga gucucccagu cuugucccgg |
| | cagugccgcc cuccccacua agaccuaggc gcaaaggcuu ggcucauggu |
| | ugacagcuca gagagagaaa gaucugaggg a |
| 64 | ucccucagau cuuucucucu cugagcuguc aaccaugagc caagccuuug |
| | cgccuagguc uuagugggga gggcggcacu gccgggacaa gacugggaga |
| | cuccaggccg guggagggaa acggcaacag gaguuuucca ccgggaauua |
| | ggggugcagg gcucugucuc gccacauacc cuucgccccg gggcaggucc |
| | gggccucaca ggcgccuccc cuggaucucu g |
| 65 | acuuuuggug ggcauuuaaa aaugugugug uauguguaua uauguaugug |
| | uauguaugug uauauaugua uauguaugua uguaucgcgu guaugugugu |
| | auguaugcau guguauguau guauaugcau guauguguau guguauauau |
| | guguguguau guauguaugu auguauaugu auuauacaca uauacacaua |
| | uugguuuuuu uaaucauuug agaguuaguu gaagauaaaa acccaucacc |
| | ccuaaaugua uuccaaagaa uaagaacauu guuuuauaca uagcacacuu |
| | aacaaaauca agaaauuuaa cauuaauaca guacuguuac cuaauccgua |
| | gucgauuuuc aaauuuuguc aguuguucca auaauguccu uuauauauuc |
| | cccgcccagc |
| 66 | gcugggcggg gaauauauaa aggacauuau uggaacaacu gacaaaauuu |
| | gaaaaucgac uacggauuag guaacaguac uguauuaaug uuaaauuucu |
| | ugauuuuguu aagugugcua uguauaaaac aauguucuua uucuuuggaa |
| | uacauuuagg ggugaugggu uuuuaucuuc aacuaacucu caaaugauua |
| | aaaaaaccaa uauguguaua uguguauaau acauauacau acauacauac |
| | auacacacac acauacacac acacauacac auacacacac auauacauac |
| | acacacauac auauauacac auacacauac augcauauac auacauacac |
| | augcauacau acacacauac acgcgauaca uacauacaua uacauauaua |
| | cacauacaua cacauacaua uauacacaua cacacacauu uuuaaaugcc |
| | caccaaaagu |
| 67 | aauuccagcg agaggcagag ggagcgagcg ggcggccggc uaggguggaa |
| | gagccgggcg agcagagcug cgcugcgggc guccugggaa gggagauccg |
| | gagcgaauag ggggcuucgc cucuggccca gcccucccgc uugauccccc |
| | aggccagcgg uccgcaaccc uugccgcauc cacgaaacuu ugcccauagc |
| | agcgggcggg cacuuugcac uggaacuuac aacacccgag caaggacgcg |
| | acucucccga cgcggggagg cuauucugcc cauuugggga cacuuccccg |
| | ccgcugccag gacccgcuuc ucugaaaggc ucuccuugca gcugcuuaga |
| | cgcuggauuu uuuucgggua guggaaaacc agcagccucc cgcga |
| 68 | ucgcgggagg cugcugguuu uccacuaccc gaaaaaaauc cagcgucuaa |
| | gcagcugcaa ggagagccuu ucagagaagc ggguccuggc agcggcgggg |
| | aagugucccc aaaugggcag aauagccucc ccgcgucggg agagucgcgu |
| | ccuugcucgg guguuguaag uuccagugca aagugcccgc ccgcugcuau |
| | gggcaaaguu ucguggaugc ggcaaggguu gcggaccgcu ggccuggggg |
| | aucaagcggg agggcugggc cagaggcgaa gcccccuauu cgcuccggau |
| | cucccuuccc aggacgcccg cagcgcagcu cugcucgccc ggcucuucca |
| | cccuagccgg ccgcccgcuc gcucccucug ccucucgcug gaauu |
| 69 | gggcacuuug cacuggaacu uacaacaccc gagcaaggac gcgacucu |
| 70 | agagucgcgu ccuugcucgg guguuguaag uuccagugca aagugccc |
| 71 | guacugacau cquagaugga aaucauaaac ugacucuugg uuugauuugg |
| | aauauaaucc uccacuggca g |
| 72 | cugccagugg aggauuauau uccaaaucaa accaagaguc aguuuaugau |
| | uuccaucuac gaugucagua c |
| SEQ ID | |
| NO: | Exemplary Target & Target-Binding Sequences |
| 73 | CTCTGATCAGCGGCGGGTGGCCTTCGGGTCATGATCTCCGCCGTGTCTAGTCCGTGGCT |
| | CACGCAGCTCTCGCACTTCTGCGACGTTGCAGCCTTCGCAGCCAGCAGTCTGAGCGGCC |
| | TGGGATCCCCGTCGCCTGGCGCCGACCCGTTCGGCCCTCGCGAGCCGCCGCCACCGCGC |
| | TACGATCCGTGCGCTGCAGTCCCCGGTGCCCCGGGCCCGCCGCCGCCGCGCGCCTATCC |
| | TTTCGCGCCCGCCCCCGGGGCGGCTGGCAGCTCGGCGGCGGAGTCCGAGGGTCCGGGGG |
| | CTAGCCGCGCGGCTGCGGTCAAGGCTCCGGTGAAGAAGAACCCGAAGGTGGCCAGCGTG |
| | AGCGTGCAGCTGGAGATGAAGGCGCTGTGGGACGAGTTCAATCAGCTGGGCACCGAGAT |
| | GATCGTCACCAAGGCAGGCAGACGAATGTTCCCCACGTTCCAAGTGAAGCTTTTTGGAA |
| | TGGATCCCATGGCCGACTACATGCTGCTCATGGACTTTGTGCCCGTAGATGACAAGCGC |
| | TACCGGTATGCTTTCCATAGCTCCTCCTGGCTGGTGGCCGGCAAGGCAGATCCTGCTAC |
| | ACCTGGCCGAGTACACTACCACCCGGACTCGCCGGCTAAGGGCGCACAGTGGATGAAAC |
| | AGATTGTGTCTTTCGACAAGCTGAAACTGACCAATAACCTGCTGGATGACAATGGCCAT |
| | ATTATTCTCAACTCCATGCACAGATATCAGCCCCGATTCCATGTTGTCTATGTGGACCC |
| | TCGAAAAGACAGTGAGAAATATGCAGAGGAGAACTTCAAAACTTTTGTGTTTGAGGAGA |
| | CACGCTTCACTGCAGTCACTGCCTACCAGAATCACCGGATCACGCAGCTTAAGATTGCC |
| | AGCAACCCCTTCGCCAAAGGCTTCCGGGATTGCGACCCGGAGGACTGGCCCCGGAACCA |
| | CCGGCCCGGAGCGCTGCCGCTCGTGAGTGCCTTTGCTCGCTCTCGGAATCCCGTGGCTT |
| | CCCCCACGCAGCCCAATGGCTCAGACAAAGACGCTGCAGAAGCCCGGCGCGAGTTCGAC |
| | CGTGACTCCGGACCCGCAGCGCTCGGCGACGCTACGCACCCGCCGCAGCTGCTGGCGCG |
| | CGTGCTGAGCCCCGCACTGCCCGGGCCTGGCGGCCTCGTCCCGCTACCCGGCGGATCCG |
| | GAGGCCGCCACAGTCCCCCGCACGCCGATCTGCGCCTGGAGGCGCCGGGCGCGTCCGAG |
| | CCGCTGCACCACCATCCCTACAAGTACCCGGCCGCCGCCTACGACCACTACCTCGGGGC |
| | CAAGAGCCGGCCGGCGCCCTACCCGCTGCCAGGCCTGCGCGGCCACGGCTACCACCCGC |
| | ACGCGCACCCGCACGCGCACCCGCACCATCACCACCACCCCGCGGTGAACCCGGCCGCC |
| | GCCGCCGCTGCTGCCGCAGCAGCCAACGTGTACTCGTCGGCGGCCGCGCCGCCCGGTGC |
| | CTACGACTACTGCCCCAGATAGTGCGCCCGCGCGCCGACCCCGAGGGCCATCCAAGGAC |
| | GCGCTCCCCATCTGGGGAGCCATGCGGGTTTCCCGCCCGCCAGTGCCAAAGCTCCCGTC |
| | CAGGCGGAAGGAAGTGGTATTTATTGTTCTCCGCGAGACCTCGTCGCCTCCGGCCCGGC |
| | CGGCAATTGCAGTGTAGACGACCGAGAGAGCCCCGCCTGCGGGCGGTGTAGATACGTGT |
| | AGATATGTGTAGATACTGTAGATACTGCACCGGCGCCGATTTCATAAACGGTTTTGCCT |
| | CTTTTGGAAATTGCCG |
| 74 | CTCTGATCAGCGGCGGGTGGCCTTCGGGTCATGATCTCCGCCGTGTCTAGTCCGTGGCT |
| | CACGCAGCTCTCGCACTTCTGCGACGTTGCAGCCTTCGCAGCCAGCAGTCTGAGCGGCC |
| | TGGGATCCCCGTCGCCTGGCGCCGACCCGTTCGGCCCTCGCGAGCCGCCGCCACCGCGC |
| | TACGATCCGTGCGCTGCAGTCCCCGGTGCCCCGGGCCCGCCGCCGCCGCGCGCCTATCC |
| | TTTCGCGCCCGCCCCCGGGGCGGCTGGCAGCTCGGCGGCGGAGTCCGAGGGTCCGGGGG |
| | CTAGCCGCGCGGCTGCGGTCAAGGCTCCGGTGAAGAAGAACCCGAAGGTGGCCAGCGTG |
| | AGCGTGCAGCTGGAGATGAAGGCGCTGTGGGACGAGTTCAATCAGCTGGGCACCGAGAT |
| | GATCGTCACCAAGGCAGACGAATGTTCCCCACGTTCCAAGTGAAGCTTTTTGGAATGGA |
| | TCCCATGGCCGACTACATGCTGCTCATGGACTTTGTGCCCGTAGATGACAAGCGCTACC |
| | GGTATGCTTTCCATAGCTCCTCCTGGCTGGTGGCCGGCAAGGCAGATCCTGCTACACCT |
| | GGCCGAGTACACTACCACCCGGACTCGCCGGCTAAGGGCGCACAGTGGATGAAACAGAT |
| | TGTGTCTTTCGACAAGCTGAAACTGACCAATAACCTGCTGGATGACAATGGCCATATTA |
| | TTCTCAACTCCATGCACAGATATCAGCCCCGATTCCATGTTGTCTATGTGGACCCTCGA |
| | AAAGACAGTGAGAAATATGCAGAGGAGAACTTCAAAACTTTTGTGTTTGAGGAGACACG |
| | CTTCACTGCAGTCACTGCCTACCAGAATCACCGGATCACGCAGCTTAAGATTGCCAGCA |
| | ACCCCTTCGCCAAAGGCTTCCGGGATTGCGACCCGGAGGACTGGCCCCGGAACCACCGG |
| | CCCGGAGCGCTGCCGCTCGTGAGTGCCTTTGCTCGCTCTCGGAATCCCGTGGCTTCCCC |
| | CACGCAGCCCAATGGCTCAGACAAAGACGCTGCAGAAGCCCGGCGCGAGTTCGACCGTG |
| | ACTCCGGACCCGCAGCGCTCGGCGACGCTACGCACCCGCCGCAGCTGCTGGCGCGCGTG |
| | CTGAGCCCCGCACTGCCCGGGCCTGGCGGCCTCGTCCCGCTACCCGGCGGATCCGGAGG |
| | CCGCCACAGTCCCCCGCACGCCGATCTGCGCCTGGAGGCGCCGGGCGCGTCCGAGCCGC |
| | TGCACCACCATCCCTACAAGTACCCGGCCGCCGCCTACGACCACTACCTCGGGGCCAAG |
| | AGCCGGCCGGCGCCCTACCCGCTGCCAGGCCTGCGCGGCCACGGCTACCACCCGCACGC |
| | GCACCCGCACGCGCACCCGCACCATCACCACCACCCCGCGGTGAACCCGGCCGCCGCCG |
| | CCGCTGCTGCCGCAGCAGCCAACGTGTACTCGTCGGCGGCCGCGCCGCCCGGTGCCTAC |
| | GACTACTGCCCCAGATAGTGCGCCCGCGCGCCGACCCCGAGGGCCATCCAAGGACGCGC |
| | TCCCCATCTGGGGAGCCATGCGGGTTTCCCGCCCGCCAGTGCCAAAGCTCCCGTCCAGG |
| | CGGAAGGAAGTGGTATTTATTGTTCTCCGCGAGACCTCGTCGCCTCCGGCCCGGCCGGC |
| | AATTGCAGTGTAGACGACCGAGAGAGCCCCGCCTGCGGGCGGTGTAGATACGTGTAGAT |
| | ATGTGTAGATACTGTAGATACTGCACCGGCGCCGATTTCATAAACGGTTTTGCCTCTTT |
| | TGGAAATTGCCG |
| 75 | CCGGTAGGGGGAGCGAGGCGGAAGGGAGCGGCGGCCGGTGCAGCCGAGGCCTCGGAGGG |
| | CACCGCCCACCGGGGCCCCAGGCCCTCGGACCGGGCGAAACTTCGCCGGCTACCAGGAT |
| | CCCCAGCCGGGATGCACTTCAGCACAGTCACCAGGGACATGGAAGCCTTCGCAGCCAGC |
| | AGTCTGAGCGGCCTGGGATCCCCGTCGCCTGGCGCCGACCCGTTCGGCCCTCGCGAGCC |
| | GCCGCCACCGCGCTACGATCCGTGCGCTGCAGTCCCCGGTGCCCCGGGCCCGCCGCCGC |
| | CGCGCGCCTATCCTTTCGCGCCCGCCCCCGGGGCGGCTGGCAGCTCGGCGGCGGAGTCC |
| | GAGGGTCCGGGGGCTAGCCGCGCGGCTGCGGTCAAGGCTCCGGTGAAGAAGAACCCGAA |
| | GGTGGCCAGCGTGAGCGTGCAGCTGGAGATGAAGGCGCTGTGGGACGAGTTCAATCAGC |
| | TGGGCACCGAGATGATCGTCACCAAGGCAGGCAGACGAATGTTCCCCACGTTCCAAGTG |
| | AAGCTTTTTGGAATGGATCCCATGGCCGACTACATGCTGCTCATGGACTTTGTGCCCGT |
| | AGATGACAAGCGCTACCGGTATGCTTTCCATAGCTCCTCCTGGCTGGTGGCCGGCAAGG |
| | CAGATCCTGCTACACCTGGCCGAGTACACTACCACCCGGACTCGCCGGCTAAGGGCGCA |
| | CAGTGGATGAAACAGATTGTGTCTTTCGACAAGCTGAAACTGACCAATAACCTGCTGGA |
| | TGACAATGGCCATATTATTCTCAACTCCATGCACAGATATCAGCCCCGATTCCATGTTG |
| | TCTATGTGGACCCTCGAAAAGACAGTGAGAAATATGCAGAGGAGAACTTCAAAACTTTT |
| | GTGTTTGAGGAGACACGCTTCACTGCAGTCACTGCCTACCAGAATCACCGGATCACGCA |
| | GCTTAAGATTGCCAGCAACCCCTTCGCCAAAGGCTTCCGGGATTGCGACCCGGAGGACT |
| | GGCCCCGGAACCACCGGCCCGGAGCGCTGCCGCTCGTGAGTGCCTTTGCTCGCTCTCGG |
| | AATCCCGTGGCTTCCCCCACGCAGCCCAATGGCTCAGACAAAGACGCTGCAGAAGCCCG |
| | GCGCGAGTTCGACCGTGACTCCGGACCCGCAGCGCTCGGCGACGCTACGCACCCGCCGC |
| | AGCTGCTGGCGCGCGTGCTGAGCCCCGCACTGCCCGGGCCTGGCGGCCTCGTCCCGCTA |
| | CCCGGCGGATCCGGAGGCCGCCACAGTCCCCCGCACGCCGATCTGCGCCTGGAGGCGCC |
| | GGGCGCGTCCGAGCCGCTGCACCACCATCCCTACAAGTACCCGGCCGCCGCCTACGACC |
| | ACTACCTCGGGGCCAAGAGCCGGCCGGCGCCCTACCCGCTGCCAGGCCTGCGCGGCCAC |
| | GGCTACCACCCGCACGCGCACCCGCACGCGCACCCGCACCATCACCACCACCCCGCGGT |
| | GAACCCGGCCGCCGCCGCCGCTGCTGCCGCAGCAGCCAACGTGTACTCGTCGGCGGCCG |
| | CGCCGCCCGGTGCCTACGACTACTGCCCCAGATAGTGCGCCCGCGCGCCGACCCCGAGG |
| | GCCATCCAAGGACGCGCTCCCCATCTGGGGAGCCATGCGGGTTTCCCGCCCGCCAGTGC |
| | CAAAGCTCCCGTCCAGGCGGAAGGAAGTGGTATTTATTGTTCTCCGCGAGACCTCGTCG |
| | CCTCCGGCCCGGCCGGCAATTGCAGTGTAGACGACCGAGAGAGCCCCGCCTGCGGGCGG |
| | TGTAGATACGTGTAGATATGTGTAGATACTGTAGATACTGCACCGGCGCCGATTTCATA |
| | AACGGTTTTGCCTCTTTTGGAAATTGCCG |
| 76 | GGTCGGTGAGGGTCGACCGGCTGTGGTCGGGCTGCGGGCGGCTCGGGCAGGTCGCGGGC |
| | GCCACAGGTGGAAGAAGAAAGGTGCCACTTTGGCATGAAGATAGACTCACTTAGACGTC |
| | AATCTTTTAAGCTGAGTGCATTGTGATTTCCAATAATTGAGGCAGTGGTTCTAAAAGCT |
| | GTCTACATTAATGAAAAGAGCAATGTGGCCAGCTTGACTAAGCCGCCAGTGTGTACAGC |
| | GCGGGCAGGACGACACCGGGTCTCGACGGACTTGTGCATGTTAGCAGTATAGATTTATG |
| | TAAGGTGGTTTAAAACTCTGGTCTTTTAAAGTAGTCTTAACTGCTCATAATATGGAGAC |
| | ATATGAGAGTCCCTCTCCTCTCCCGCGTGAGCCCGCAGGAGAAGCGATGATGGAGAACC |
| | GAGCTTGCCCCTTCCAAGTGCTGCCCCATGAACAGTCTCCACCACCTCCCCTGCAAACG |
| | TCCAGTGATGCAGAGGTAATGGACGTTGGCTCTGGTGGTGATGGACAGTCCGAACCTCC |
| | TGCCGACGACCCATTCAACTTCTACGGAGCTTCTCTTCTCTCCAAAGGATCCTTCTCTA |
| | AGGGCCGCCTCCTCATAGACCCGAACTGTAGTGGCCACAGCCCGCGCACTGCCCGGCAC |
| | GCACCTGCGGTCCGGAAGTTCTCCCCTGACCTTAAGTTGCTTAAGGATGTAAAGATTAG |
| | CGTGAGCTTTACTGAGAGCTGCAGGAGTAAGGACAGGAAGGTGCTGTACACAGGAGTAG |
| | AACGCAGCACTCGGCCTGAGTGTGGCCAGCTCCTTAGTCCTGTCAGTGGGGACGTGCAT |
| | GCTTGTCCCTTTGGCGGGAGTGTTGGTAATGGGGTAGGCCTAGGGGGTGAGAGTGCAGA |
| | TAAGAAGGATGAGGAAAATGAGCTGGATCAGGAAAAGAGAGTGGAGTATGCAGTGCTCG |
| | ATGAGTTAGAAGATTTTACTGACAATTTGGAGCTAGATGAAGAAGGAACAGGCGGGTTC |
| | ACGGCTAAAGCAATCGTTCAAAGAGACAGAGTGGATGAAGAGGCCTTGAATTTCTCCTA |
| | TGAGGATGACTTTGACAATGATGTGGACGCTTTACTAGAAGAAGGTCTCTGTGCTCCCA |
| | AGAAGAGGCGAATGGAGGAAAAATATGGCGGAGACAGTGATCATCCATCTGATGGAGAG |
| | ACAAGTGTACAGCCAATGATGACCAAGATTAAAACAGTGCTCAAAAGTCGTGGCCGTCC |
| | ACCTACAGAGCCATTGCCTGATGGATGGATCATGACTTTTCATAATTCTGGAGTCCCTG |
| | TATACCTGCACAGAGAGTCTCGAGTGGTCACTTGGTCCAGACCCTACTTCTTGGGAACA |
| | GGAAGCATACGGAAACATGATCCTCCTCTAAGCAGTATCCCCTGCCTACATTATAAGAA |
| | AATGAAGGACAATGAGGAACGAGAACAAAACTGTGATCTTGCCCCCAGTGGAGAGGTGT |
| | CACCTGTCAAGCCCTTGGGTCGGTCTGCAGAGTTGGATTTCCCTCTGGAAGAGCCTGAC |
| | TCCATGGGTGGAGACTCAGGGTCCATGGATGAGAAGGACCCATTGGGGGCTGAGGCAGC |
| | CGCTGGAGCCCTGGGACAAGTGAAGGCTAAAGTTGAGGTGTGCAAAGATGAATCAGTTG |
| | ATCTGGAGGAATTTCGTAATTACCTTGAGAAGCGTTTTGACTTTGAACAAGTAACTGTG |
| | AAAAAATTCAGGACTTGGGCTGAGCGGCGTCAGTTCAACCGTGAGATGAAGCGGAAGCA |
| | GGCCGAGTCAGAGAGGCCCATCCTGCCAGCCAACCAGAAGCTGATCACTCTATCTGTAC |
| | AAGATGCACCCACAAAGAAAGAGTTTGTCATCAATCCCAATGGGAAGTCTGAGGTTTGC |
| | ATCCTGCACGAATACATGCAGCGTGTCCTCAAGGTCCGCCCTGTTTATAATTTCTTTGA |
| | ATGTGAGAATCCAAGTGAGCCTTTTGGTGCCTCCGTGACCATTGATGGTGTGACTTACG |
| | GATCTGGAACTGCAAGCAGCAAAAAACTTGCGAAGAATAAAGCTGCCCGAGCCACCCTG |
| | GAAATTCTCATCCCTGACTTTGTTAAACAGACCTCTGAGGAGAAGCCTAAAGACAGTGA |
| | AGAACTGGAGTATTTTAACCACATCAGTATTGAGGATTCACGAGTCTATGAGCTGACAA |
| | GCAAGGCTGGGCTGTTGTCTCCATATCAGATCCTCCATGAGTGCCTTAAAAGAAACCAT |
| | GGAATGGGTGACACATCCATCAAGTTTGAAGTGGTTCCTGGGAAAAACCAGAAGAGTGA |
| | ATATGTTATGGCATGCGGCAAACACACAGTGCGCGGGTGGTGTAAGAATAAACGAGTTG |
| | GGAAACAATTAGCATCTCAGAAAATCCTTCAGCTACTGCACCCACATGTCAAGAACTGG |
| | GGTTCCTTACTACGCATGTATGGTCGTGAGAGCAGCAAAATGGTCAAGCAGGAGACCTC |
| | TGACAAGAGTGTGATAGAGCTACAGCAGTATGCCAAGAAGAACAGGCCCAACCTTCACA |
| | TCCTGAGCAAGCTACAAGAAGAGATGAAGAGGCTGGCTGCAGAGCGGGAGGAGACTCGG |
| | AAGAAACCCAAGATGTCAATTGTAGCATCTGCCCAGCCTGGTGGTGAGCCCTTGTGCAC |
| | AGTCGATGTATGAGGTAGGCAGCATGGGCCAACAGTGCTACCCAGGAGAGACCATCAGC |
| | CACACATCATCACTCTGCAGCTCCAGGCCTCCAACCTAAGTTCCTTCCCTGTGGCAGGG |
| | TCTGGGCTCTGGCTCTGGCACATGGGGACAGCTGTGACTACACAGTACACATGCAAAGA |
| | AGCAGTTCTGGACAAGCTGCTCTCAATGTGACTGGATATACTTAAGGATCAGTCATAAG |
| | TTAACTGCACAAGTGGAAGCTGATAGACAGCTGTAGATCCTGCTTGTATGTATCTGCTG |
| | CGGACCGTTTTTATGAAGGTTTTCATTAATTTTAGTACACTATATGCACTGACAAGAAG |
| | TAATACTTCGTTGATAGATGAGATGACCAGGTTTTATAGCTTTGGCTGGCCTATGATGC |
| | CAGGTAGCCCCTTTGCTATGGTTCTTTCTGGTGGAGCCACTGAGACTTGGTCAGAAGAT |
| | GCAGGTATCCTGTCCTGTAGAATTACCTGCTTTGTGTGTTTTGCATTTTCTTCTGAAAA |
| | AGTTATAACAGAAAGGAATATTTCAGGCTATTTTGGCTTAAAATAAAACAAAATTTTAG |
| | CACCAGAAATACTCTTCCGAGATTGCAGTGAAGATACTGAGTGTTAGTCCGATTGATCC |
| | TTTTTCTTATGAAGTCTTAATGTCCTAACAGCCGTCTTCTGGATACTGAAGCTTCCACC |
| | TTCGATAAACTTAGCAACAATTTTTTACAGAGATTTTTGCACAACCAAGCCCATCTGTT |
| | CATCAATTTTTAAAGCTTTTATGATGTTTTAAAATTTGGAAAGAAACTTTTACAGTAAG |
| | AATGAAAGATGCATTTCAACAGATGAATATGCATAAATAGGGAACTGGCCTTCAATGTA |
| | GCCCATCACTGACCAAGAGCTGCCTGGCACATTGTACACGATAAGTAGTTATGGTGTTT |
| | TTTGATCTCAAGGATAGCTGCTCTGTGCAGCCAGGCCTGATTGCAGGTTTTTGTCTACC |
| | TATGCTCATTGGATAGCCCCTGTAAATGGGTATTGAGTAGAGGGGTATTCAGAGCCCTA |
| | TTACCTGTTCAGAGGGGCTACACTCTACATCCTCAGTGTCTCTAACCATGTACCTGAAG |
| | ATACATGGGTGACATGTGCTGTTAGAAAGATAAGATGGCAACTGTGGGCCTCCCAACAG |
| | CATCTTCCTCCTCCTTAATCATGTGTGTGACAGCAGTTAATGTTTATGTTAATCTTGGC |
| | AAAAAGAACCCTTAATTCAAGTTGGTAGACTTGGACACTTGATTCTTTTCTGCCTCCCA |
| | TTCAGTTGTGTTCAGTCTTTTGACTAACCATTGCCCTTAAAACATACTTAACATTGAAA |
| | AAGCATATGGTAATGGTGGTATCTCTGCAGCCTCACCGACCCAGGCACTGTCAACAGGT |
| | GCTGCCACACATTCCTGGTCTCCAGTGCCCTCCCTGTCTCTTACAGGTGGGTCTTATAT |
| | GTACAGTCATGCTGCAAGGGTGGGGAGCCTGTGCTGATGTGCTTCTGTGTGTTGGCCAA |
| | GGCTGTAGGGTGTACCCTGATGGTCTGAACACATAAA |
| 77 | GCAGCCCTGCTAGAAGCAGGTCCCCTCCTAATCAGAACCCTGGCCTGTGTGGGCACGCC |
| | CACGCGGACACGTTCCAGGACAGCCCCGCCACCGCGACCCCCTGTGGACACTCGCACAG |
| | GCACCCTCTCCGCGTGCACCGCCCACGACCCCACCCTACCTCCCGCTGCACTCGCCGCG |
| | CCCCTTGTCCCGCGTGTCAGAGCCCGTGTCCGCGGCCGGAAGCGCCTGCTGGGTCCACG |
| | CAGCGCCGCCACCATCGCCGCCACCATCGCCGCCATGGTCCGGGGACCTCAGGCGGTCA |
| | GGGCTGCCCTTCGCACTGCCTGAGCGTCCGCGCCTCGGGGTCAGACCTGTGCGCGGCCT |
| | TGACGAGGGGATGAGAGAGTCCTACCACAGTGAAACTCAAAGTTACAGACATTCTGGCC |
| | CATAAATGCTGTTGGCTGCTGTCTCATTGGGTCTCCTGTTGCTGGCCTTCCTCCTGCTC |
| | CTGCGACACCTAGGCTGGGGCTTGGTGGCTATTGGTTGGTTTGAGTTCGTGCAGCAGCC |
| | GGTCCACAACCTGCTCATGGGTGGCACAAAGGAGCAGCGCATCCTGCGCCATGTGCAGC |
| | AACACGCAAAGCCTGGAGACCCCCAGAGCGTCCTGGAGGCCATTGATACCTACTGCTCA |
| | GAGAAGGAGTGGGCCATGAACGTGGGTGACGCAAAAGGCCAAATCATGGATGCAGTGAT |
| | TCGGGAGTACAGGCCCTCGCTGGTGCTGGAGCTAGGAGCTTATTGTGGCTACTCAGCCG |
| | TGCGAATGGCCCGCCTGCTGCCACCTGGAGCCAGGCTTCTCACCATGGAGATTAACCCT |
| | GACTACGCTGCCATCACCCAGCAAATGCTGGACTTCGCAGGCCTACAGGACAAAGTTTC |
| | CATCCTCATCGGGGCATCCCAGGACCTTATCCCCCAGCTGAAGAAGAAGTACGATGTGG |
| | ACACATTAGACATGGTCTTTCTTGACCACTGGAAAGACCGCTACCTTCCAGACACACTT |
| | CTCCTGGAGGAATGTGGCCTGCTGCGCAAGGGGACGGTGCTCCTAGCTGACAATGTCAT |
| | TGTCCCGGGAACCCCTGACTTCCTGGCGTATGTGAGGGGGAGCAGCAGCTTCGAGTGCA |
| | CACACTACAGCTCATACCTGGAGTACATGAAAGTGGTGGACGGCTTGGAGAAGGCAGTC |
| | TACCAGGGTCCAGGCAGCAGCCCCGTGAAGTCCTGACCACTCAGCCTGATGAGCTTCCG |
| | TCCCAGCTCCCTTCTGCACGATGACACACACTCACTCTGACCCCCTCTATGCTTCTGGG |
| | GCCTTTCCTCAGGGCCTGTGGCTCCAGATTGTCATACACTGGCACATTAAAGGTAGTGA |
| | GCTCACCATGCAAACCACTACAATACCCCTGGAAAACACCTGT |
| 78 | GCGGTGTTTTCCTGCTCTCTGCTCCGCCTGGCCTATGTGTTTACATCTATGAAAACGCC |
| | CCGCTCTTCTAATAAATGCGCCCGTCTGGCCCCCTATCCTTCACCAGCCTAGTTCTTTT |
| | ATGCATGCAATCGAGCTCAGGGCCCGCTGCCGTCTGCCGCTAGATGCATGCTGGCGCCC |
| | GCCCGGCTGGCGCTCGCGAGATGCCCAATAGCGCGAATTGATGGATGGCGGAAGCTAGT |
| | TACTAAGCTGGACTCGGAGCCGCGGAGGTAGAACTAGAGATCTGCACACCACCCTGGTT |
| | CTGCATCGCCGGGCGGGATCTCAAGGCTTTGCCAGCCTGCTCACACCGACCCTCATCGG |
| | CGACCACATCAGTGGCAGCGGAGTATCCCCGTCCTCCCCCCCCTGTCTACCGAAGAACC |
| | GAGGCGGCCCGGTCCGCGTAGGGAGCGGTTTCCCGCCGGGGCGATTTGGCAGGTGCGCG |
| | CCGTGACTTCCGGCGTTGCCCGGGAGCCGCCGGAGGAGGAGCGGCGCAGGGGATGCGGC |
| | TGTGGCGGCGGCGGCGGCGGCCGAGCGCAGGAGGCGGCTGTGGCGGCGGCTGGGGGCAC |
| | GGGCCGGCGATGGCGCGGCGGCGCTGAGGGCAAGGGTGGGCGGCGGCCGGAGGGCGGGC |
| | GGCGCGGGAGGAAGCTGCGGCAGCTCCATGGCCCAGGCGTGCTGAGGGACACGGCTCTG |
| | GCTTCAGCCCGGCAGCGGCCGAACAATGAAGCTCTTGAAGCCAACCTGGGTCAACCACA |
| | ATGGGAAGCCAATTTTTTCAGTTGATATTCACCCTGATGGGACCAAGTTTGCAACTGGA |
| | GGACAAGGGCAGGATTCTGGGAAGGTTGTGATCTGGAATATGTCTCCAGTCCTCCAGGA |
| | GGATGACGAGAAGGATGAAAATATTCCCAAGATGCTTTGCCAGATGGACAATCACTTAG |
| | CATGTGTGAACTGTGTGCGGTGGTCAAACAGTGGGATGTATTTAGCTTCTGGGGGAGAT |
| | GACAAACTGATTATGGTGTGGAAGCGGGCTACGTACATTGGGCCCAGCACTGTGTTTGG |
| | TTCCAGTGGTAAGCTTGCCAATGTGGAGCAATGGCGGTGTGTCTCCATCCTCCGGAGTC |
| | ACTCAGGCGATGTGATGGATGTAGCATGGTCTCCCCACGACGCCTGGCTGGCCTCATGC |
| | AGCGTGGATAACACTGTTGTCATTTGGAATGCCGTGAAGTTCCCAGAAATTCTTGCAAC |
| | TCTGAGAGGTCATTCTGGCTTAGTAAAAGGTTTGACTTGGGATCCCGTTGGTAAATATA |
| | TTGCCTCTCAAGCTGATGATCGAAGTTTGAAGGTATGGAGGACGCTGGACTGGCAGCTA |
| | GAGACTAGCATCACCAAGCCTTTTGATGAGTGTGGAGGAACGACGCATGTTCTCCGGCT |
| | TAGTTGGTCACCTGATGGCCATTACCTGGTATCTGCCCATGCCATGAATAATTCTGGCC |
| | CCACTGCTCAGATCATCGAAAGAGAGGGCTGGAAGACCAACATGGACTTTGTGGGTCAC |
| | CGGAAAGCTGTGACTGTTGTGAAATTCAACCCAAAAATCTTCAAGAAGAAGCAGAAGAA |
| | TGGGAGCTCTACAAAGCCCAGCTGCCCATACTGCTGCTGTGCTGTTGGCAGCAAGGACC |
| | GCTCACTCTCTGTCTGGCTCACATGTTTGAAACGGCCTCTGGTTGTCATCCATGAACTG |
| | TTTGACAAGTCCATCATGGATATTTCCTGGACTCTGAATGGGTTGGGTATCCTGGTGTG |
| | CTCCATGGACGGCTCTGTGGCGTTCCTTGATTTCTCTCAGGATGAACTCGGAGACCCCC |
| | TGAGTGAGGAGGAAAAGAGCCGAATTCACCAGTCTACCTATGGCAAGAGCCTGGCAATA |
| | ATGACTGAGGCCCAGCTTTCCACAGCTGTTATTGAGAACCCTGAGATGCTCAAGTACCA |
| | GCGGAGACAGCAACAGCAGCAGCTGGATCAGAAGAATGCCACTACTAGGGAGACAAGCT |
| | CAGCATCCTCAGTCACGGGTGTGGTCAATGGGGAAAGTCTAGAAGATATCAGAAAGAAT |
| | CTTTTGAAGAAACAAGTGGAAACTCGGACAGCAGATGGTCGGAGGAGAATCACGCCTCT |
| | TTGCATAGCACAGCTGGACACTGGGGACTTCTCCACGGCATTCTTCAACAGCATCCCAC |
| | TCTCCAGCTCCCTAGCAGGCACCATGCTCTCCTCTCCTAGTGGTCAGCAGCTACTACCA |
| | CTGGACTCCAGTACCCCCTCCTTCGGCGCCTCAAAGCCTTGCACAGAACCAGTGGCAGC |
| | CACCAGTGCCAGGCCTACAGGCGAATCTGTCAGTAAGGACAGTATGAATGCTACCTCTA |
| | CTCCTGCTGCATCGTCACCCTCTGTGTTAACAACCCCATCCAAGATTGAACCCATGAAA |
| | GCATTTGATTCCCGGTTCACAGAACGGTCCAAAGCCACACCAGGTGCTCCTTCCTTGAC |
| | CAGTGTGATTCCAACAGCTGTTGAAAGGTTGAAAGAGCAGAACCTCGTCAAGGAGCTGA |
| | GGTCCCGGGAACTGGAGAGCAGCAGTGACAGCGATGAGAAGGTCCACCTAGCCAAGCCC |
| | TCTTCACTGTCCAAGCGCAAACTTGAGCTTGAGGTAGAGACGGTGGAAAAGAAGAAGAA |
| | AGGTCGCCCTAGGAAGGATTCACGTCTTTTACCCATGTCTCTGTCCGTCCAGTCTCCAG |
| | CTGCCCTGTCTACAGAGAAGGAGGCCATGTGTCTGTCTGCACCAGCACTTGCACTGAAG |
| | CTGCCAATTCCAGGCCCACAGAGAGCGTTCACCCTCCAGGTGAGCTCTGACCCCTCCAT |
| | GTACATTGAGGTGGAGAATGAAGTGACCACCGTTGGGGGGATAAGGCTGAGTCGCCTGA |
| | AGTGCAACCGTGAAGGGAAGGAGTGGGAGACAGTGCTCAGCAGTCGCGTCCTCACTGCT |
| | GCCGGCAGCTGTGATGTGGTATGTGTTGCCTGTGAAAAGAGGATGCTGTCGGTGTTCTC |
| | TACCTGTGGTCGCCGTCTCCTCCCTCCCATCCTCCTTCCATCTCCAATCTCTACTTTGC |
| | ACTGCACGGGCCCCTACGTCATGGCACTCACCGCTGCAGCCACACTGTCTGTCTGGGAT |
| | GTTCACAGACAGGTGGTTGTGGTGAAAGAAGAATCTCTACACTCCATCTTGTCAGGAAG |
| | TGATATGACGGTGTCACAGATCTTGCTAACACAGCATGGAATCCCAGTGATGAATTTGT |
| | CTGATGGGAAAGCATACTGCTTTAATCCATCCCTCTCCACATGGAACCTGGTTTCTGAC |
| | AAGCAAGATTCATTGGCCCAGTGTGCAGACTTCAGGAACAGCCTGCCGTCCCAGGATGC |
| | CATGCTTTGTTCAGGACCGTTAGCCATAATTCAGGGCCGCACCTCCAACTCTGGAAGGC |
| | AAGCTGCCCGGCTCTTCTCCGTGCCTCATGTGGTACAGCAGGAGACCACACTGGCCTAC |
| | CTAGAGAACCAGGTTGCTGCGGCACTTACCCTGCAGTCAAGCCACGAGTATCGTCACTG |
| | GCTCCTCCTTTATGCTCGGTACCTTGTGAATGAAGGGTTTGAATACCGCCTCCGTGAAA |
| | TATGCAAGGACTTGCTGGGTCCAGTTCACTGCTCCACTGGAAGTCAGTGGGAGTCAACA |
| | GTAGTGGGTCTGCGGAAAAGGGAGCTGCTGAAGGAACTGTTGCCAGTCATTGGGCAGAA |
| | TCTTCGATTCCAGCGCCTCTTTACTGAGTGCCAGGAACAACTGGACATCCTGAGAGACA |
| | AGTAGTCTGTCCTGGCTTGCCTCAGCTGCTGCAAGGGCAGGACCACACTATTGCCACTG |
| | GCAGGTTGCCCAGGGCTAGCCCCACCTGTCAGGGCGGTAGTAGGAAGAGAGACCTTGGC |
| | AGAAGATGTGTTGTCCTGCACCAGCACTAGCCCAGTTCCCTGGGTGGACATGGACTATA |
| | TCCCAAGCCTCCATGACTCCAAAAGGGGGCAGCTTGTCCCTCCATGTAGGCCCAGCTGT |
| | GGACTTGGGGCTGGGACTGGGGCTCTGCCTTGCTGCCAGCAGGGATCTCTTGAGCCTGT |
| | CTCCAGCTTGACCCCAAGGTAGACACTGGCATCTGCCACTGCAGGTGCTGTGCTGCTTC |
| | AGCTGTAACGAATGAGCCATTTGTGAGAGCAGGAGCCGGGGAGTGTTAGGGATGATGCA |
| | GTCGGCAGACGGACCCGAAGCCTGTGTGAATGCTAAGCTGTTCTGACACATGGACTATT |
| | TTTGTACTAGAATTTGCTAACTTGGAATATGGAGTTTCTGTTGGTTGATCAGGATATCC |
| | CTATAAGTTACTTGGACATTGGTCACTTGTAGGAAATTTAAACTCTAATTATGACAGCT |
| | ACACTGAAAAAAAAATAATTGTACTGAAATTAACTTGTCTATCTTCATTTGGTTTAATT |
| | TTTAAATGTTTGTAAAAAGAGACACTTTGTGTGGGGGGGGGGGGGACAGAGGGGAGG |
| | GCAATTTCTTTTCTAAGTGTAAAATAAATGAACATGCATTGGATACCAAAAAAAAAAAA |
| | AAAAAAAAAA |
| 79 | TTAAGCCTCCTGAGCGCGTGTATCGGCTCCGCCATGGCTCTCAAGCGCGTCTTCTTGCT |
| | GCGGTCGGTGGCACCACGCGTCGCTGCCCTCTCAACCAAACCGCAAGCCCAGGAACAGC |
| | CTCCCGCGAGCCCTGAGGCTCTTCGGGGATGTGGGGCGGCCAAGGCTGTGCGGCCGCCT |
| | GTGCCAGCCGTGGACTTCACCAACACGCAGGAGGCGTATCGCAGCCGGCGGAGTTGGGA |
| | GTTGGTGCGCAACCTGCTAGTGCTGCGGCTGTGTGCGTCGCCGGTGCTGCTAGCGCACC |
| | ACGAGCAGTTGTTCCAAGTTGCCAGGAAGCTTCTGGGGCAAAGGATGTTCGAGAGATTG |
| | ATGAAGATGACCTTCTATGGCCATTTTGTGGCTGGCGAGGACCAGGAGTCTATCAGGCC |
| | TCTGATCCGGCACAACAAAGCCTTTGGTGTTGGCTTTATCCTGGACTATGGAGTGGAGG |
| | AAGATCTGAGCCCTGAGGAGGCGGAGCGCAAAGAGATGGAGTCATGCACTTCTGAAGCA |
| | GAGAGAGATGGCAGTGGAGCAAATAAGAGGGAGAAGCAGTATCAGGTGCACCCCGCCTT |
| | TGGAGACCGCAGAGATGGTGTCATCAGTGCCCGCACCTACTTCTATGCCAATGAAGCCA |
| | AGTGTGACAACTACATGGAGAACTTACTGCAGTGCATCAAGGCCTCAGGTGGAGCCAGT |
| | GATGGTGGTTTCTCAGCCATTAAGCTCACTGCACTGGGGAGACCACAGTTTCTGCTGCA |
| | GTTCTCAGACGTGCTGACCAGGTGGAGACGGTTCTTCCATCAAATGGCTGCAGAGCAGG |
| | GACAGGCTGGGCGTGCTGCTGTAGACACAAAGCTGGAGGTGGCGGTGCTCCAGGACAGC |
| | ATCGCAAAGATGGGCATCGCATCCAGGGCTGAGATTGAAGGGTGGTTCACGCCAGAGAC |
| | GCTGGGAGTGTCTGGCACCGTGGACTTGCTGGACTGGAACAGCCTCATTGACAGCAGGA |
| | CCCGGCTCTCCAGGCACTTGGTGGTCCCCAATGTGCAGACTGGCCAGCTGGAGCCCCTG |
| | CTGTCACGGTTCACTGAGGAGGAAGAGCAGCAGATGAAAAGGATGCTGCAGAGGATGGA |
| | TGTACTGGCCAAGAAAGCAAAAGAGGCAGGTGTGCGCCTGATGATTGATGCTGAGCAGA |
| | GCTACTTCCAACCAGCCATCAGCCGCCTGACCCTGGAGATGCAGCGCAGGTTCAATGTG |
| | GATAAGCCGTTCATCTTCAACACATTCCAGTGCTACCTCAAGGATGCCTATGACAATGT |
| | GACCTTGGATATGGAACTGGCTCGCCGTGAGGGCTGGTGTTTTGGGGCCAAGCTGGTAC |
| | GTGGTGCATACATGGCCCAAGAGCGTGTCAGGGCAGCAGAGATCGGTTATGAGGACCCC |
| | ATCAACCCTACATATGAAGCCACCAATGCTATGTACCACAGGTGCCTTAACTATGTTCT |
| | GGAGGAGCTGAAGCACAGCACCAAGGCAGAGGTGATGGTGGCTTCCCACAACGAGGACA |
| | CCGTGCACTTCACGTTGTGCAGGATGAAGGAGATAGGCCTGCATCCTGCTGATGGTCAG |
| | GTGTGCTTCGGACAGCTGCTGGGGATGTGTGACCAAATCAGCTTCCCACTAGGCCAGGC |
| | AGGCTTTCCTGTGTACAAGTATGTGCCCTATGGCCCTGTGATGGAGGTACTCCCTTACC |
| | TGTCCCGCCGTGCCCTGGAGAACAGCAGCATCATGAAGGGTGCTCAGCGAGAGAGGCAG |
| | CTGCTATGGCAGGAGCTCCGCAGGCGGCTGCGCACTGGCAGCCTCTTCCACCATCCGGC |
| | CTAGTCACCGCAGGAGCCTTGCCCACCCGCTCGTACTCCACTCAACCCCTTACCTCTGG |
| | GGCTTCAGGCGGGGCACAGCTTGGGATTGGGCTGGGGTTCCTTAGCCCAACCTGCCCAG |
| | ACACAGTTCACCTTTTTATGCCCAAGGCTTTTTATGCCCAAGGCGGGATTTCATCAGTG |
| | GACAGCTCCTGAGGAACAGTGCCCAAGATGGTCGTCTGGTCACAGAGGCTGCCCTCTGG |
| | GACTTCCTGTACCCCAAGGAACAGACACTCAGGAGTGGGGTCAGCTAGAGCCCCTGGGA |
| | GCTGCCCCACTAATCTGAGTAAGCACTGACCACCTCTGCAGGTTACAGAGCCCTAGTCC |
| | AGGATTAACCTTCTGCCAGGGTCTAACCCATCTTCCCTGCACTGGGCAGAGGACAGACT |
| | AGGAAGCCTGTTTAGTCAATAAATCATCCTGTAACAGAGTC |
| 80 | GGGATTTCCTCCACTGCCGCTGCGGCGGATCCTGCGCCGCGTCCAGCCCGCGCGCCCGA |
| | CCCCGGCCCGACCCGGCCGGCCCCGCCCGCCCGGCCCGGGGAGGGATGCGGCGGCGCGG |
| | CGCCCAGGATGCCCCGCAGCCCCGGGACGCGCCTCAAACCCGCCAAGTACATCCCGGTG |
| | GCCACGGCCGCCGCGCTGTTGGTTGGTTCCAGCACACTCTTCTTCGTATTCACGTGCCC |
| | ATGGTTGACAAGAGCTGTGTCTCCAGCTATTCCTGTCTACAATGGCATCCTCTTCCTCT |
| | TTGTCCTGGCCAACTTCAGTATGGCTACCTTCATGGACCCTGGAGTCTTCCCCCGAGCG |
| | GACGAGGACGAGGACAAGGAGGATGACTTCCGGGCCCCACTGTACAAGAATGTGGATGT |
| | GCGGGGCATCCAGGTCCGCATGAAGTGGTGTGCAACGTGCCACTTTTACCGTCCACCGC |
| | GCTGCTCACACTGCAGTGTCTGTGACAACTGTGTGGAGGACTTTGACCATCACTGCCCC |
| | TGGGTCAACAACTGCATTGGACGCCGCAACTACCGTTACTTCTTCCTGTTCCTGCTGTC |
| | ACTCAGCGCGCACATGGTGGGGGTGGTGGCCTTCGGCCTGCTCTATGTGCTCAATCACT |
| | CGGAGGGGCTGGGAGCCGCCCACACCACCATCACCATGGCTGTCATGTGTGTGGCTGGC |
| | CTTTTCTTCATTCCTGTCATCGGCCTCACTGGCTTTCACGTGGTACTGGTCACGCGGGG |
| | CCGCACCACCAATGAGCAGGTGACTGGGAAGTTCCGCGGGGGTGTGAATCCCTTCACCC |
| | GAGGCTGCTATGGGAACGTGGAGCACGTGTTATGCAGTCCCCTGGCGCCCCGGTATGTG |
| | GTGGAACCCCCCAGGATGCCGCTCTCAGTGAGCCTCAAGCCACCCTTCCTGAGACCTGA |
| | GCTCCTGGAACGAGCTGTGCCCCTCAAGGTCAAACTTAGCGACAATGGGCTGAAAGCTG |
| | GCCGCAGCAAGTCCAAGGGCAGTCTAGACCAGCTGGATGAGAAACCTCTGGACCTGGGA |
| | CCTCCACTGCCCCCCAAGATAGAGGCTGGTACCTTTGGAAGAGATCTGAAGACCCCAAG |
| | ACCTGGCAGTGCTGAGAGTGCCCTATCAGTACAGAGGACCAGCCCCCCAACACCTGCCA |
| | TGTATAAGTTCCGGCCAGCCTTCTCCACTGGTCCCAAGACACCCTTTTGTGGACCTAAT |
| | GAGCAGGTCCCAGGTCCTGACTCCCTTACTCTGGCAGATGACAGCACCCACAGTCTAGA |
| | CTTTGTGTCTGAGCCCAGCCTGGATCTCCCAGACCACGGGCCGGGTGGTCTGCGTCCTC |
| | CCTACCCGCCCTCCCCACCCCTCAACACCACTGATGCCTTCTCAGGTGCCTTGCGCTCC |
| | CTGAGTCTCAAGGCTGCCAGTCGGAGGGGTGGGGACCACATGACCTTACAGCCACTGCG |
| | CTCTGAAGGTGGGCCCCCTACACCTCACCGTAGTCTCTTTGCTCCTCATGCACTGCCCA |
| | ATCGAAATGGCAGCCTGTCATATGACAGCCTACTTAACCCTGGCTCACCCAGTGGCCAC |
| | GCATGCCCCACACACCCCTCTGTTGGTATAGCCAGCTACCATTCACCCTACCTGCACCC |
| | TGGGCCATCAGATCCACCGCGGCCCCCACCCCGCAGCTTCAGCCCTGTGCTGGGTCCCC |
| | GGCCTAGGGAACCCTCTCCTGTGCGCTATGACAACCTGTCTCGGACCATCATGGCCTCT |
| | ATCCAGGAGCGCAAGGACAGGGAAGAGCGTGAACGGCTGCTGCGCTCCCAGACTGACTC |
| | ACTCTTTGGCGACTCTGGTGTCTATGATACACCCAGCTCCTACAGCCTGCAACAGGCCA |
| | GTGTGTTAACGGAAGGCCCCCGTGGCTCTGTCCTGCGTTACGGCTCCAGGGATGACCTC |
| | GTGGCCGGGCCTGGCTTTGGTGGTGCCCGCAATCCTGCCCTGCAGACGTCATTGTCCTC |
| | GCTGTCCAGCTCCATGAGTCGGGCACCTCGGACATCTTCCTCCTCCCTGCAGGCTGACC |
| | AGGCCAACAACAATGCCCCAGGACCCCGGCCTGGCAGTGGTTCACATAGGTCACCTGCC |
| | CGTCAGGGCCTGCCTTCCCCACCAGGCACCCCCCGATCGCCCTCCTACACGGGCTCCAA |
| | GGCCGTCGCCTTCATCCACACAGACCTCCCGGACCGGCAGCCCTCACTGGCTATGCAGA |
| | GGGATCACCCTCAGCTGAAGACCCCCCCAAGTAAGCTTAACGGGCAGTCCCCAGGCATG |
| | GCCCGTCTGGGGCCTGCTGCCAGCCCCATGGGGCCCAACGCCAGCCCTGCCCGGCACAC |
| | GCTGGTTAAGAAGGTGTCCGGCGTGGGTGGGACTACGTATGAAATCTCGGTGTGAGAAC |
| | TGACCACCACTCATCTGCTGTGATGCCACGGGGACCAGGACCCCCCGCAGTAGCTCCCC |
| | AACCCTATCAAGTTCTCTGCCCCAGGGAGGAGGCTCCCCAAGCCTGCTGTGGACACATC |
| | AACAGGAAAGACAGCCTTGCCTCCACAAGCTTGAGCTCCGGCCTCTGCCTGTTCATCTA |
| | CTTACCTAGTACTCACAGGGCAGATGGGTCCAAGGCTGTGGGCTGTGATGGGCACCTGG |
| | ACTGCCTCCTTCATCAGCCAAGCTCAGCCCTGCATCCTATTGCCCTCATTCCCTAAGGC |
| | CAGTCCCCAGCAGTCCCATGGGCCCACCCACTCCTGTCTGGATGGGTTGCTCAGCCCTG |
| | TTCTCCAGCTGCTCCTAACTCACGGCCTTTCACTGACAAAGCTGGTCGCTTGTTGCTTG |
| | GCAAGGCCTGTGCTTTGCAGGGAGCATAACCCTGGCCTGCTCCGGGTTGGGATGCATAG |
| | ATGAACGTACGGACAGATACTTGAGCGGTAGCAGGGGTCCCAGCTCCCTCTGTTCGTGT |
| | CTTGCCTGGCTGGTGGGTTCGTGTCCCTGTGTCTGTATGCTGTGCTGCCGTGCCACGTC |
| | TGATGTGTTAGTGCTCGGCTGCCACTGTTCTTTCATCAAAGCCTTAACTTCTGCTTTAT |
| | GCTCTTGTGGGAGGTGATGGGGGGTGGGGTCGGCCAGAGCAGAGAGGGGGCCAGATGCT |
| | GCCCGGGAGACCAGTGGGGCCGAGAGCCCCCTCCCTCTCTCTCGGGCCTTCCCTGCCAA |
| | ACTGGAGAATCCTCACCCCAAAGCATCCCGGGTTCCCAGCACTGGCTTGGCCAGCACAG |
| | CTGGAAAGCACATCAGGGCGGGTCCATTCGTCCCCTGGCGTCAGGGGCCTGAGTAAGCA |
| | CATGGCAGCGTCCGCTTGCATCGTGTCTGTTCTATGTTTTTATATTTACATCTATATAT |
| | CTATAATTTTATTAAAAAAAGGAAAAATCATTTTGAGCCTTTCCTTGGGCAAGGCTGTG |
| | GCTGTGGGTACAGGGGTGTCTCAGGAAAGGGGTTGGCCAGCTGGTGCAGGGTGGCATGT |
| | TCCTCTTGGACAGCTCTGCTGGCATTGCCAAGTCAGGCCTAGTTGGTAACGAATGGACT |
| | TCTGCTGGTGAGTTGCAAAGGTGTCTTACTCCCAGTCTGTGTCATGCCGGACGGAAGGG |
| | TGTTTGGTGAAAGGCTAGGACCTCAGCTGCCTCCTGGGACTCTGAGGTACCAAAGGGCT |
| | TGGAGCAGCTATATAGTGGAGGCTGCACATGAGGCCAATGCCTGGCCGAGAAACCTACA |
| | GATACTACTATACTTCTTCCCTGCCATATGCCAAGGGAGGAATTAGGGAGTGTCAGGAA |
| | GAACTCAGAGCAGGGCTGGAAAGGGCTAGGGTAGGTCCAGAGTTTCAAGAAGGGGAGGT |
| | GTCCAGAGCCTGGGGCCCCTTAGTTCTACCCACTGCTGACTTCCACCTTTCTGCTTCTC |
| | TCTCATGGATATCGGCCAACAGGGGGCGCATTGGCAGCTGTAGCCGTGGATGGGGGTGG |
| | CGTGGTACGCCTAGGGGGACTCCTGGCCTCCACCTATGCCACCTTGGCCACCCTGAGGA |
| | TCACCCGCCGCTGCGGGCCCCCTGGAGCGTGGCTGCTGGGGCAGCCCCCCGTGGGACCA |
| | TTTGTCGGCTACATTCTGCTGCATCCAGCCTTTTCCCCAGCCTCTCAGGTCCCTAACAG |
| | GACACTAGTAGGTGGCTCTAGGGCCATGGACAGTCTTCTGGGGACCCAGAAGACGAGGT |
| | TGCTGTAGAGCTTTGGAGTTGGGCTGGATGTAGCCTGGAGATCCCTACCCCGTTCCCCG |
| | TCAACCAGCCACATCTTGACTGATCAGTCTGCCAGGGTCCACGACCCTGTCTCCAGGTG |
| | ACCTCAGGAAGTCCCCCCACCTGCTGCAGGGGTCCAGTGCCCCAATGGGGGTGGCCTCA |
| | GAGCTACTGGGAACCAGCATGACCTTTACCCCAACCTCCTTACCCCTTCCAAGCACTTT |
| | AAGCCACTTCCTCTAGGGAGCCCAGGCCTCTGTGGGTTGGGCTGGGGTGGGGGTCTCAG |
| | GTCACCGCAGGTTACTGCACTCCTCCACTCAACCCCAAGCCCCTGAACAGATGCCTTAA |
| | AGTCCTGGAGCTGCCAGCCAGCTTGGTGCACCATGAACCTGGTCCACCCGTCTCTGCCT |
| | CTTGCCTGCTCTGACCCACCTGGGTCTTCTGCAGCCAAGGGTGATACCTCTGGGAATAC |
| | CCAAGCCTGTGGGCCTGGATACCCCTGGGAGGAGTCAGAACAGAACCCCTGCCAACTCC |
| | TCTTTTCACACAGGGGCTGTGTCCTGGGGGCTACTTCTGGGCAGCAGTTATGTGTCTCC |
| | CATATGGGGACAGATCACAAACTGTTTTCTGTTGTATAGGATTTCCCTCCCTCAACTTC |
| | CCTAATAAATTTTTTTTTTGTTCACCTCCAAAAAAAAAAAAAAAAAAAA |
| 81 | GGGGGGCGGAGGGAAGAGCGGACGGGCGGGAGCGCCGGCGCCAGACGCGGAGGAAAGGA |
| | GCTGCGACTAGCCGCCCAGAGGCCGCCGAGCCAGCGACGCCTGAGCTAGTCGAGCCACC |
| | GTCGCCGCGCCCCCATGGCGGCCGCCAAGGACAGTCACGAGGACCATGATACTTCCACA |
| | GAGAATGCAGATGAGTCCAACCACGACCCCCAGTTCGAGCCAATAGTTTCTCTTCCCGA |
| | GCAAGAAATTAAAACGCTGGAGGAAGATGAAGAGGAACTTTTTAAGATGCGTGCAAAGC |
| | TGTTCCGGTTTGCTTCAGAGAATGACCTCCCAGAATGGAAGGAGCGAGGCACTGGAGAT |
| | GTCAAGCTTCTGAAGCACAAGGAGAAAGGGACCATCCGCCTTCTTATGAGGAGGGACAA |
| | AACCTTGAAGATATGCGCCAACCACTATATTACACCAATGATGGAGCTGAAGCCGAATG |
| | CTGGCAGTGACCGAGCCTGGGTCTGGAATACCCACGCCGACTTTGCTGACGAGTGCCCC |
| | AAGCCTGAGCTGCTCGCCATCCGCTTCCTAAATGCTGAGAATGCACAAAAGTTCAAAAC |
| | AAAGTTTGAAGAATGCAGGAAAGAAATTGAAGAGAGAGAAAAGAAAGGACCAGGCAAAA |
| | ATGATAATGCCGAAAAGGTGGCCGAGAAGCTGGAAGCCCTTTCAGTGAGGGAGGCCAGA |
| | GAGGAGGCTGAAGAGAAGTCTGAGGAGAAACAATGAATCACTCTGTCTTTTTCCTTTCC |
| | TTTTCTTTTTAAAAATTTGCCCTACCCTTTAAGGTTTGTTTTTCTGTTTTGTTTTTACA |
| | AGGGACTTTATAAAGAACTGAATTCC |
| 82 | TCACCCCTCAGCCGGCCTCGGCCTCCACCGCTGGTCGCCGCGCCCCGCCCGCGCGCCCG |
| | CCGCCACACGTCCCCCGCCGGCGGCCACCATGAGCACAGGACTGCGGTACAAAAGCAAG |
| | CTGGCGACCCCAGAGGACAAACAGGACATCGACAAGCAGTACGTTGGCTTCGCCACACT |
| | GCCCAACCAGGTGCACCGCAAGTCCGTCAAGAAAGGTTTCGACTTCACGCTCATGGTGG |
| | CCGGTGAGTCCGGCCTGGGGAAGTCCACCCTTGTCCATAGCCTCTTTCTGACCGACCTG |
| | TATAAGGACCGGAAACTGCTGAGTGCTGAGGAACGCATCAACCAGACGGTAGAGATCCT |
| | GAAACACACCGTCGACATTGAGGAGAAGGGGGTCAAGTTAAAGCTCACCATTGTGGACA |
| | CGCCCGGCTTTGGGGACGCGGTGAACAACTCTGAATGTTGGAAGCCCATCACTGACTAT |
| | GTGGACCAGCAGTTTGAGCAGTATTTCCGTGATGAGAGTGGCCTGAACCGCAAGAACAT |
| | CCAGGACAACCGGGTACACTGCTGCCTGTACTTCATCTCCCCGTTCGGACACGGACTGA |
| | GGCCAGTGGATGTAGGCTTCATGAAGGCACTGCATGAGAAGGTGAACATCGTCCCACTC |
| | ATCGCCAAAGCTGACTGCCTGGTGCCCAGTGAGATCCGGAAGCTGAAGGACAGAATACG |
| | TGAGGAGATCGACAAGTTTGGGATCCACGTGTACCAGTTTCCAGAATGTGATTCGGATG |
| | AAGATGAAGACTTCAAGCAACAGGACCGGGAACTGAAGGAAAGTGCACCCTTCGCCGTT |
| | ATTGGCAGCAACACTGTGGTGGAGGCCAAGGGGCAGCGGGTCCGGGGGCGACTGTACCC |
| | CTGGGGGATCGTCGAAGTGGAGAATCAGGCGCACTGCGACTTTGTGAAGCTCCGCAACA |
| | TGCTCATCCGCACTCACATGCACGACCTCAAAGATGTGACGTGCGACGTGCACTATGAG |
| | AACTACCGTGCCCACTGCATCCAGCAGATGACCAGCAAACTCACCCAGGACAGCCGCAT |
| | GGAGAGCCCCATTCCTATCCTCCCACTACCCACACCGGATGCGGAGACCGAGAAGCTCA |
| | TCAGGATGAAGGATGAAGAGCTAAGGCGCATGCAGGAGATGTTGCAGAAGATGAAGCAG |
| | CAAATGCAAGACCAGTGACACCCGCCCCAGCCCCACGTCGTCGACAAGGATAGACGGCC |
| | GGTTTCCGGGCTGGCCCCTCCTACCCCTGGATCCCAGACTGTCCTGGATTCCACCCTGG |
| | GTTCATCTGGATCTCAGAAGGCCTGGACCTCACCCTAATCCAAAGTGGCTTTGACCAGA |
| | CTGTTCAGACCTGGAGCCACAGAGCCACAGCCCCCAGATGACCCTAATTTATTCTCGGC |
| | GTCCACCCTTCCCGGTCATTTGTATCTGCTTCCGAGTGCTCTGAATCACAGCCCCTCCC |
| | CAACCTCCTGCCCCCGCCCCACCCCGCACCTCCCCGCCTATCATAAGGGAACAAGATGT |
| | GCACAACCTCCAAGATCTCCCTTCCTAGAAGATCACTGCCCCCCAGCGGGCCAATAAAC |
| | CAGGGTAGAGGAGGGACGTGGATGCAGGATCTTGGGCCTATTACCCAAGCTAGTGCTGC |
| | AGAGTGGAGTTGGGAGGCCCCCCTCTGCCGATTCCAGTGGGCTACGAAGCATTTGCTAA |
| | TGGCCTACTGAGCGCGGAAGTAGGCCGGTTCCTCCCTTACTCCCTAATCATGCTCTGTT |
| | CAGGATCGGAGGCGGCGGGGTTTGGAGGCTAAGGCCTTTAGGAAGACCCCAGATCTAGA |
| | GGCTTCTGGGAGGAAGGCGGGGCCGCGATGCTCTAGACCGCTCGCGCCCCTTCCTGAGT |
| | CTACCTGAAGGACTCCAAGGGCGGTAGAGACGGGGTGTCCCTATGCCTAGACTACTTTC |
| | GTCCACCCATAGGTCAAGGTCTTTTCTTCCCAGCAACTCTGTTGCACAAGGATTCCAGC |
| | CCTTGGCCTCCTCCATATCTCCACCCGCATGATTCCTCCCCACACACCCCATGCTCCGT |
| | TTTGTTCAGTTGTGAATGCCGCGTCCTGTCCTGGTGACAGGAGAACAAAGTTGGTGAAC |
| | GTCAAAAAAAAAAAAAAAAAAAAAAAAAAAA |
| 83 | ATCCTCTCCTGGCCCGCGCTGCGAGCGCCCCGCCAGTCCGCGCCGCCGCCCTCACCCTG |
| | TGCGCCCGCAGCCCGCGAGCCCAGCCCGGCCCGGTAGAGCGGAGCGCCGGAGCCTCGTC |
| | CCGCGGCCGGGCCGGGACCGGGCCGGAGCAGCGGCGCCTGGATGCGGACCCGGCCGCGC |
| | GCAGACGGGCGCCCGCCCCGAAGCCGCTTCCAGTGCCCGACGCGCCCCGCTCGACCCCG |
| | AAGATGAAGAGGGCGTCCTCCGGAGGAAGCAGGCTGCTGGCATGGGTGTTATGGCTACA |
| | GGCCTGGAGGGTAGCAACACCATGCCCTGGTGCTTGTGTGTGCTACAATGAGCCCAAGG |
| | TAACAACAAGCTGCCCCCAGCAGGGTCTGCAGGCTGTGCCCACTGGCATCCCAGCCTCT |
| | AGCCAGCGAATCTTCCTGCATGGCAACCGAATCTCTCACGTGCCAGCTGCGAGCTTCCA |
| | GTCATGCCGAAATCTCACTATCCTGTGGCTGCACTCTAATGCGCTGGCTCGGATCGATG |
| | CTGCTGCCTTCACTGGTCTGACCCTCCTGGAGCAACTAGATCTTAGTGATAATGCACAG |
| | CTTCATGTCGTGGACCCTACCACGTTCCACGGCCTGGGCCACCTGCACACACTGCACCT |
| | AGACCGATGTGGCCTGCGGGAGCTGGGTCCCGGCCTATTCCGTGGACTAGCAGCTCTGC |
| | AGTACCTCTACCTACAAGACAACAATCTGCAGGCACTCCCTGACAACACCTTTCGAGAC |
| | CTGGGCAACCTCACGCATCTCTTTCTGCATGGCAACCGTATCCCCAGTGTGCCTGAGCA |
| | CGCTTTCCGTGGCCTGCACAGTCTTGACCGCCTCCTCTTGCACCAGAACCATGTGGCTC |
| | GTGTGCACCCACATGCCTTCCGGGACCTTGGCCGCCTCATGACCCTCTACCTGTTTGCC |
| | AACAACCTCTCCATGCTGCCTGCAGAGGTCCTAATGCCCCTGAGGTCTCTGCAGTACCT |
| | GCGACTCAATGACAACCCCTGGGTGTGTGACTGCCGGGCACGTCCACTCTGGGCCTGGC |
| | TGCAGAAGTTCCGAGGTTCCTCATCAGAGGTGCCCTGCAACCTGCCCCAACGCCTGGCA |
| | GACCGTGATCTTAAGCGCCTCGCTGCCAGTGACCTAGAGGGCTGTGCTGTGGCTTCAGG |
| | ACCCTTCCGTCCCATCCAGACCAGTCAGCTCACTGATGAGGAGCTGCTGAGCCTCCCCA |
| | AGTGCTGCCAGCCAGATGCTGCAGACAAAGCCTCAGTACTGGAACCCGGGAGGCCAGCT |
| | TCTGCCGGAAACGCCCTCAAGGGACGTGTGCCTCCCGGTGACACTCCACCAGGCAATGG |
| | CTCAGGCCCTCGGCACATCAATGACTCTCCATTTGGAACTTTGCCCAGCTCTGCAGAGC |
| | CCCCACTGACTGCCCTGCGGCCTGGGGGTTCCGAGCCACCAGGACTTCCCACCACTGGT |
| | CCCCGCAGGAGGCCAGGTTGTTCCCGGAAGAATCGCACCCGCAGCCACTGCCGTCTGGG |
| | CCAGGCGGGAAGTGGGGCCAGTGGAACAGGGGACGCAGAGGGTTCAGGGGCTCTGCCTG |
| | CTCTGGCCTGCAGCCTTGCTCCTCTGGGCCTTGCACTGGTACTTTGGACAGTGCTTGGG |
| | CCCTGCTGACCAGCCACCAGCCACCAGGTGTGTGTACATATGGGGTCTCCCTCCACGCC |
| | GCCAGCCAGAGCCAGGGACAGGCTCTGAGGGGCAGGCCAGGCCCTCCCTGACAGATGCC |
| | TCCCCACCAGCCCACCCCCATCTCCACCCCATCATGTTTACAGGGTTCCGGGGGTGGCG |
| | TTTGTTCCAGAACGCCACCTCCCACCCGGATCGCGGTATATAGAGATATGAATTTTATT |
| | TTACTTGTGTAAAATATCGGATGACGTGGAATAAAGAGCTCTTTTCTTAA |
| 84 | CTCTGATCAGCGGCGGGTGGCCTTCGGGTCATGA |
| 85 | GTGGCCTTCGGGTCATGA |
| 86 | AGGCAGACGAATGTTCCCCACGTTCCAAGTGAAGCTTTTTGGAA |
| 87 | AGGCAGACGAATGTTCCC |
| 88 | AGGGACACGGCTCTGGCTTCAGCCCGGCAGCGGCCGAACAATGA |
| 89 | GCAGCGGCCGAACAATGA |
| 90 | TTGTGATCTGGAATATGT |
| 91 | AAAACTCTGGTCTTTTAAAGTAGTCTTAACTGCTCATAATATGG |
| 92 | CTTAACTGCTCATAATATGG |
| 93 | CCGCAGGAGAAGCGATGA |
| 94 | TTAAGCCTCCTGAGCGCGTGTATCGGCTCCGCCATGG |
| 95 | GTATCGGCTCCGCCATGG |
| 96 | ATGGCTCTCAAGCGCGTC |
| 97 | GCGACGCCTGAGCTAGTCGAGCCACCGTCGCCGCGCCCCCATGG |
| 98 | GTCGCCGCGCCCCCATGG |
| 99 | ATGGCGGCCGCCAAGGAC |
| 100 | CGCCCGGCCCGGGGAGGGATGCGGCGGCGCGGCGCCCAGGATGC |
| 101 | GCGCGGCGCCCAGGATGC |
| 102 | ATGCCCCGCAGCCCCGGG |
| 103 | GCCGCTTCCAGTGCCCGACGCGCCCCGCTCGACCCCGAAGATGA |
| 104 | GCTCGACCCCGAAGATGA |
| 105 | ATGAAGAGGGCGTCCTCC |
| 106 | ATGCTGTTGGCTGCTGTCTCATTGGGTCTCCTGTTGCTGG |
| 107 | ATGCTGTTGGCTGCTGTC |
| 108 | TTGGTTTGAGTTCGTGCAGCAGCCGGTCCACAACCTGCTCATGG |
| 109 | TCCACAACCTGCTCATGG |
| 110 | TCATGACCCGAAGGCCACCCGCCGCTGATCAGAG |
| 111 | TCATGACCCGAAGGCCAC |
| 112 | TTCCAAAAAGCTTCACTTGGAACGTGGGGAACATTCGTCTGCCT |
| 113 | GGGAACATTCGTCTGCCT |
| 114 | TCATTGTTCGGCCGCTGCCGGGCTGAAGCCAGAGCCGTGTCCCT |
| 115 | TCATTGTTCGGCCGCTGC |
| 116 | ACATATTCCAGATCACAA |
| 117 | CCATATTATGAGCAGTTAAGACTACTTTAAAAGACCAGAGTTTT |
| 118 | CCATATTATGAGCAGTTAAG |
| 119 | TCATCGCTTCTCCTGCGG |
| 120 | CCATGGCGGAGCCGATACACGCGCTCAGGAGGCTTAA |
| 121 | CCATGGCGGAGCCGATAC |
| 122 | GACGCGCTTGAGAGCCAT |
| 123 | CCATGGGGGCGCGGCGACGGTGGCTCGACTAGCTCAGGCGTCGC |
| 124 | CCATGGGGGCGCGGCGAC |
| 125 | GTCCTTGGCGGCCGCCAT |
| 126 | GCATCCTGGGCGCCGCGCCGCCGCATCCCTCCCCGGGCCGGGCG |
| 127 | GCATCCTGGGCGCCGCGC |
| 128 | CCCGGGGCTGCGGGGCAT |
| 129 | TCATCTTCGGGGTCGAGCGGGGCGCGTCGGGCACTGGAAGCGGC |
| 130 | TCATCTTCGGGGTCGAGC |
| 131 | GGAGGACGCCCTCTTCAT |
| 132 | CCAGCAACAGGAGACCCAATGAGACAGCAGCCAACAGCAT |
| 133 | GACAGCAGCCAACAGCAT |
| 134 | CCATGAGCAGGTTGTGGACCGGCTGCTGCACGAACTCAAACCAA |
| 135 | CCATGAGCAGGTTGTGGA |
| SEQ ID | |
| NO: | Exemplary spacer sequence |
| 136 | GATGCGGCCGCCACTGTGCTGGATATCTGCAGAATTCGCCCTT |

Since the invention encompasses both RNAs and DNAs, It will be understood that any of the sequences disclosed herein may refer equally to both an RNA and a DNA. In instances where a sequence comprises Uracil nucleotides (and may therefore be considered to represent an RNA sequence) it will be understood that said sequence will also represent a corresponding DNA sequence (e.g., a DNA sequence encoding said RNA sequence) in which each Uracil is replaced with a Thymine, but that is otherwise identical in sequence, and vice versa.

1. A functional nucleic acid molecule comprising:
two or more target binding sequences, wherein each target binding sequence comprises a sequence reverse complementary to a target mRNA sequences for which protein translation is to be enhanced; and
a regulatory sequence comprising a SINE B2 element or a functionally active fragment thereof, or an internal ribosome entry site (IRES) or a functionally active fragment thereof.

2. (canceled)

3. The functional nucleic acid molecule of claim 1, wherein the two or more target binding sequences are separated by a spacer.

4. The functional nucleic acid molecule of claim 3, wherein the spacer is 19 nucleotides in length.

5. The functional nucleic acid molecule of claim 1, wherein the functional nucleic acid molecule is a trans-acting functional nucleic acid molecule.

6. The functional nucleic acid molecule of claim 1, wherein the regulatory sequence is orientated, within the functional nucleic acid molecule, in the direct orientation relative to the 5′ to 3′ orientation of the functional nucleic acid molecule.

7. The functional nucleic acid molecule of claim 1, wherein the regulatory sequence is located 3′ of the two or more target binding sequences.

8. The functional nucleic acid molecule of claim 1, wherein the target binding sequences are complementary to target mRNA sequences encoding two or more of TBX-1, HIRA1, DGCR8, PRODH, COMT, RANBP1, ZDHHC8, SEPT5 and RTN4R.

9. The functional nucleic acid molecule of claim 1, wherein the target binding sequences are complementary to target mRNA sequences as set forth in any two or more of SEQ ID NOs: 73-109, or a fragment thereof.

10. The functional nucleic acid molecule of claim 9, wherein the target binding sequences are complementary to target mRNA sequence which has at least about 80%, identity to a sequence selected from the group consisting of SEQ ID NOs 73-109, or a functionally active fragment thereof.

11. The functional nucleic acid molecule of claim 8, wherein the target binding sequences are complementary to target mRNA sequences encoding DGCR8, TBX1 and COMT.

12. The functional nucleic acid molecule of claim 11, wherein the target binding sequences are complementary to target mRNA sequences as set forth in any two or more of SEQ ID NOs: 73-77, 84-87, 91-93, and 106-109, or a fragment thereof.

13. The functional nucleic acid molecule of claim 12, wherein the target binding sequences are complementary to target mRNA sequences as set forth in any two or more of SEQ ID NOs: 93, 87, and 108.

14. The functional nucleic acid molecule of claim 13, wherein the target binding sequences comprise SEQ ID NOs: 119, 113, and/or 134.

15. The functional nucleic acid of claim 1, wherein the regulatory sequence comprises a SINE B2 element or a functionally active fragment of a SINE B2 element.

16. (canceled)

17. The functional nucleic acid molecule of claim 1, wherein the SINE B2 element comprises a sequence which has at least about 80%, identity to a sequence selected from the group consisting of SEQ ID NOs 2-54, or a functionally active fragment thereof.

18. The functional nucleic acid molecule of claim 15, wherein the SINE B2 element comprises a sequence selected from the group consisting of SEQ ID NOs 2-54, or a functionally active fragment thereof.

19. (canceled)

20. The functional nucleic acid molecule of claim 15, wherein the fragment is about 10 nucleotides in length.

21. The functional nucleic acid of claim 1, wherein the regulatory sequence comprises an IRES.

22. (canceled)

23. The functional nucleic acid molecule of claim 21, wherein the IRES comprises a sequence which has at least about 80% identity to a sequence selected from the group consisting of SEQ ID NOs 55-72, or a functionally active fragment thereof.

24. The functional nucleic acid molecule of claim 21, wherein the IRES comprises a sequence selected from the group consisting of SEQ ID NOs 55-72, or a functionally active fragment thereof.

25. (canceled)

26. The functional nucleic acid molecule of claim 21, wherein the fragment is about 10 nucleotides in length.

27. The functional nucleic acid molecule of claim 17 or, wherein identity is defined across the length of overlap between the SINE B2 element or the IRES sequence and the sequence selected from the group consisting of SEQ ID NOs 2-54 or 55-72, respectively.

28. The functional nucleic acid molecule of claim 1, wherein the functional nucleic acid molecule comprises RNA nucleotides or modified RNA nucleotides.

29. (canceled)

30. The functional nucleic acid molecule of claim 1, wherein the functional nucleic acid molecule is single stranded.

31. A DNA molecule encoding the functional nucleic acid molecule of claim 1.

32. An expression vector comprising the DNA molecule of claim 31.

33-37. (canceled)

38. A method of treating a disease associated with gene defects comprising administering the functional nucleic acid molecule of claim 1 to a subject.

39. (canceled)

40. The method of claim 38, wherein the disease associated with gene defects is a microdeletion.

41. The method of claim 40, wherein the microdeletion is a microdeletion of part of chromosome 22.

42. The method of claim 41, wherein the microdeletion is 22q11.2DS.

43. An in vitro method for enhancing translation of one or more target mRNA sequences, comprising administering the functional nucleic acid molecule of claim 1 to a cell or a cell-free system.


SEQUENCE LISTING (136 sequences)

SEQ ID NO: 1 - RNA, 13 nt, synthetic construct
  source 1..13: mol_type = other RNA
atctgcagaattc

SEQ ID NO: 2 - RNA, 167 nt, synthetic construct
  source 1..167: mol_type = other RNA
cagtgctagaggaggtcagaagagggcattggatcccccagaactggagttatacggtaacctcgtggtggttgtgaaccaccatgtggatggatattgagttccaaacactggtcctgtgcaagagcatccagtgctcttaagtgctgagccatctctttagctcc

SEQ ID NO: 3 - RNA, 77 nt, synthetic construct
  source 1..77: mol_type = other RNA
gaactggagttatacggtaacctcgtggtggttgtgaaccaccatgtggatggatattgagttccaaacactggtcc

SEQ ID NO: 4 - RNA, 29 nt, synthetic construct
  source 1..29: mol_type = other RNA
cctcgtggtggttgtgaaccaccatgtgg

SEQ ID NO: 5 - RNA, 60 nt, synthetic construct
  source 1..60: mol_type = other RNA
gttatacggtaacctcgtggtggttgtgaaccaccatgtggatggatattgagttccaaa

SEQ ID NO: 6 - RNA, 98 nt, synthetic construct
  source 1..98: mol_type = other RNA
atcccccagaactggagttatacggtaacctcgtggtggttgtgaaccaccatgtggatggatattgagttccaaacactggtcctgtgcaagagcat

SEQ ID NO: 7 - RNA, 129 nt, synthetic construct
  source 1..129: mol_type = other RNA
gaagagggcattggatcccccagaactggagttatacggtaacctcgtggtggttgtgaaccaccatgtggatggatattgagttccaaacactggtcctgtgcaagagcatccagtgctcttaagtgc

SEQ ID NO: 8 - RNA, 183 nt, synthetic construct
  source 1..183: mol_type = other RNA
gggcagtgctagaggaggtcagaagagggcattggatcccccagaactggagttatacggtaacctcgtggtggttgtgaaccaccatgtggatggatattgagttccaaacactggtcctgtgcaagagcatccagtgctcttaagtgctgagccatctctttagctccagtctcttaagct

SEQ ID NO: 9 - RNA, 67 nt, synthetic construct
  source 1..67: mol_type = other RNA
gaactggagttatacggtaacctcgtggtggttgtgaaccaccatgtggatggatattgagttccaa

SEQ ID NO: 10 - RNA, 107 nt, synthetic construct
  source 1..107: mol_type = other RNA
ggatcccccagaactggagttatacggtaacctcgtggtggttgtgaaccaccatgtggatggatattgagttccaaacactggtcctgtgcaagagcatccagtgc

SEQ ID NO: 11 - RNA, 127 nt, synthetic construct
  source 1..127: mol_type = other RNA
agagggcattggatcccccagaactggagttatacggtaacctcgtggtggttgtgaaccaccatgtggatggatattgagttccaaacactggtcctgtgcaagagcatccagtgctcttaagtgc

SEQ ID NO: 12 - RNA, 38 nt, synthetic construct
  source 1..38: mol_type = other RNA
ggtaacctcgtggtggttgtgaaccaccatgtggatgg

SEQ ID NO: 13 - RNA, 180 nt, synthetic construct
  source 1..180: mol_type = other RNA
cagtgctagaggaggtcagaagagggcattggatcccccagaactggagttatacgataacctcgtggtggttgtgaaccaccatgtggatggatattgagttccaaacactggtcctgtgcaagagcatccagtgctcttaagtgctgagccatctctttagctccagtctcttaagct

SEQ ID NO: 14 - RNA, 180 nt, synthetic construct
  source 1..180: mol_type = other RNA
cagtgctagaggaggtcagaagagggcattggatcccccagaactggagttatacgctaacctcgtggtggttgtgaaccaccatgtggatggatattgagttccaaacactggtcctgtgcaagagcatccagtgctcttaagtgctgagccatctctttagctccagtctcttaagct

SEQ ID NO: 15 - RNA, 180 nt, synthetic construct
  source 1..180: mol_type = other RNA
cagtgctagaggaggtcagaagagggcattggatcccccagaactggcgttatacggtaacctcgtggtggttgtgaaccaccatgtggatggatattgagttccaaacactggtcctgtgcaagagcatccagtgctcttaagtgctgagccatctctttagctccagtctcttaagct

SEQ ID NO: 16 - RNA, 180 nt, synthetic construct
  source 1..180: mol_type = other RNA
cagtgctagaggaggtcagaagagggcattggatcccccagaagtggagttatacggtaacctcgtggtggttgtgaaccaccatgtggatggatattgagttccaaacactgctcctgtgcaagagcatccagtgctcttaagtgctgagccatctctttagctccagtctcttaagct

SEQ ID NO: 17 - RNA, 180 nt, synthetic construct
  source 1..180: mol_type = other RNA
cagtgctagaggaggtcagaagagggcattggatcccccagatggtgagttatacggtaacctcgtggtggttgtgaaccaccatgtggatggatattgagttccaaacacgtcacctgtgcaagagcatccagtgctcttaagtgctgagccatctctttagctccagtctcttaagct

SEQ ID NO: 18 - RNA, 180 nt, synthetic construct
  source 1..180: mol_type = other RNA
cagtgctagaggaggtcagaagagggcattggatcccccagaactgcactatacggtaacctcgtggtggttgtgaaccaccatgtggatggatattgagttccaaatgagtggtcctgtgcaagagcatccagtgctcttaagtgctgagccatctctttagctccagtctcttaagct

SEQ ID NO: 19 - RNA, 116 nt, synthetic construct
  source 1..116: mol_type = other RNA
gggcattggatcccccagaactggagttatacggtaacctcgtggtggttgtgaaccaccatgtggatggatattgagttccaaacactggtcctgtgcaagagcatccagtgctc

SEQ ID NO: 20 - RNA, 77 nt, synthetic construct
  source 1..77: mol_type = other RNA
ggactggagttatacggtaacctcgtggtggttgtgaaccaccatgtggatggatattgagttccaaacactggtcc

SEQ ID NO: 21 - RNA, 77 nt, synthetic construct
  source 1..77: mol_type = other RNA
gaactggcgttatacggtaacctcgtggtggttgtgaaccaccatgtggatggatattgagttccaaacactggtcc

SEQ ID NO: 22 - RNA, 107 nt, synthetic construct
  source 1..107: mol_type = other RNA
gatgccttagaagtggagttaagagttgtgagctgccgttttttggttctgggactcgaactcgtttcctctgatactatcaaccaccaagccatctcttcagcccc

SEQ ID NO: 23 - RNA, 131 nt, synthetic construct
  source 1..131: mol_type = other RNA
gccagaagaagttgtgggattccctggaactggagcaaccaacagtttgtgtgcaccatgtgggtaatgggaatcgaacctgggtcctctataagactggccagtgctcttaactactgaggtgcatttct

SEQ ID NO: 24 - RNA, 187 nt, synthetic construct
  source 1..187: mol_type = other RNA
ttattttaaatatatgagtatttcacctgcataggcgcacagtacccacagagactagaagagggtggcagatctcctgagactggagttaatgcttgtgagctgccatgtggatgctggaaatcaaacccaggtcctttggaaggcaggcaggtgctcttaatcatggaagcatctcttcagctcc

SEQ ID NO: 25 - RNA, 131 nt, synthetic construct
  source 1..131: mol_type = other RNA
cagcgacatcagaagaggatattggatcccattacagatggttgaaggccaccatgtcgttgctgggaatgaactcaagacctctggaagagcagtcagtgctcttaacctctgagccatctctccagccc

SEQ ID NO: 26 - RNA, 114 nt, synthetic construct
  source 1..114: mol_type = other RNA
atcccctccaaagctcaagatggttgtaagccaccctgtgattgctgggatttgaactcaagacctccggaagagcaattagtgctcttaaccgctgagcaatctctccagccc

SEQ ID NO: 27 - RNA, 357 nt, synthetic construct
  source 1..357: mol_type = other RNA
gtgcagtgctagaggaggtcagaagagggcattggatcccccagaactggagttatacggtaacctcgtggtggttgtgaaccaccatgtggatggatattgagttccaaacactggtcctgtgcaagagcatccagtgctcttaagtgctgagccatctctttagctccttattttaaatatatgagtatttcacctgcataggcgcacagtacccacagagactagaagagggtggcagatctcctgagactggagttaatgcttgtgagctgccatgtggatgctggaaatcaaacccaggtcctttggaaggcaggcaggtgctcttaatcatggaagcatctcttcagctcc

SEQ ID NO: 28 - RNA, 532 nt, synthetic construct
  source 1..532: mol_type = other RNA
gtgcagtgctagaggaggtcagaagagggcattggatcccccagaactggagttatacggtaacctcgtggtggttgtgaaccaccatgtggatggatattgagttccaaacactggtcctgtgcaagagcatccagtgctcttaagtgctgagccatctctttagctccgtgcgaattcggtgcagtgctagaggaggtcagaagagggcattggatcccccagaactggagttatacggtaacctcgtggtggttgtgaaccaccatgtggatggatattgagttccaaacactggtcctgtgcaagagcatccagtgctcttaagtgctgagccatctctttagctccgtgcgaattcggtgcagtgctagaggaggtcagaagagggcattggatcccccagaactggagttatacggtaacctcgtggtggttgtgaaccaccatgtggatggatattgagttccaaacactggtcctgtgcaagagcatccagtgctcttaagtgctgagccatctctttagctcc

SEQ ID NO: 29 - RNA, 228 nt, synthetic construct
  source 1..228: mol_type = other RNA
tttttttaaaaatttatttttattttatgtgtatgagtgttttgcctgcatgtatgtctgtgtaccacgtgcgtgcctggtgcccgcggaggccagaagagggcgtcggatcccctggaactggagttacagatggttgtgagccgccatgtgggtgctgggaatcgaacccgggtcctctggaagagcagccagtgctcttaaccgctgagccatctctccagcccc

SEQ ID NO: 30 - RNA, 214 nt, synthetic construct
  source 1..214: mol_type = other RNA
ttttttttacttgtataggtgttttgcctgcatgtgtatctatctatgtaccgaatatgttcctggtatccacagagaccaaaagtggatgttgtatctcctgaaattggagtcatagacagttatgagctgccatttgagtgcttggaatagaacccaggtcctcttaaagagcatccagtgctcttaaaaactgagacatctctgtagcctc

SEQ ID NO: 31 - RNA, 200 nt, synthetic construct
  source 1..200: mol_type = other RNA
tttattttgctttatgtgtctgagtgtttgcttgaatgtatgtctgtgtaccacgcctgtaccttgtgccttcagagttgagaggagggcataggatctcctggaactggaattgcaggtggttgtgagccaccctgtgggtcctggggaccatactccagcaagaacatcatgtgctcttaattcctgagtctccaacc

SEQ ID NO: 32 - RNA, 214 nt, synthetic construct
  source 1..214: mol_type = other RNA
tttatttacttatctttatgtgtatgagtgtgttgtcagactgttatgtctgtgtgtcacatgcatgcctgctgttcatggagtccagaagagggcatcggatcccctggaactggagttacagatgagtggccatgtgaatgttaagaaccaaacctgggtcctctgaaagagcagacaatgctcttaactactgagctgtctctccagcccc

SEQ ID NO: 33 - RNA, 205 nt, synthetic construct
  source 1..205: mol_type = other RNA
ttattttattcgtgtaagtgttttgccagcatctatgtcttcgcactatgtgcaggtctggtgcctgaggggtccagacgagagcactgggtctccgggaactggagttacagatcattgtgagccaccatgtgggtgcagggaatcgaacctgggacctctggaggagcagccactgctcttaaccactacactatttctccag

SEQ ID NO: 34 - RNA, 121 nt, synthetic construct
  source 1..121: mol_type = other RNA
tctgtggaccactgtgtacagaagcctgagaaggctagcagatccccagaactggaactgtgagacgctgtgctatggaggtgctaggaactgaaaatggatgggtcctctgcaagagcag

SEQ ID NO: 35 - RNA, 191 nt, synthetic construct
  source 1..191: mol_type = other RNA
ttgttttaattgaatggctatagggtgtttcttctgtatgtatatctatgtttggtacctacagaggcatcagatcctctggaactgtagttgctgacagttgtgagctgtcatggggatgctggaattgaacctggatcctatgaaagaacagccagtgttcttaaccgctgagctatctctccaggccc

SEQ ID NO: 36 - RNA, 205 nt, synthetic construct
  source 1..205: mol_type = other RNA
ttttttttttaattttaaaaaaaaagattttatttatttattttatatatgatgagtacactgtcactcttttcagacaccctagaaaaggggggcatcagatcccattacagatggttgtgagccacatggttgctgggaattgacctcaggacctctgaaagagcagtcagtgctctcaacctttgagtcatctctccagccc

SEQ ID NO: 37 - RNA, 190 nt, synthetic construct
  source 1..190: mol_type = other RNA
atgtatatctgtaatgggacatactcacatacatgggcacgtgagtataaaaggccagaagagagcactggaccctctggagttgagattctaagcagttgtgaaccatctgatgtaggtgctgggaactgaacttgggtcctttgctagagaagtatgtctcttaaccactgagccgtatctccatccc

SEQ ID NO: 38 - RNA, 169 nt, synthetic construct
  source 1..169: mol_type = other RNA
taaagatttattcattaagtacactgtagctatcttcagacgcatcagaagagggcgtcagatctctttacaggtggttgtgagccaccatgtggttgctggaatttgaactcaggaccttcaaaagagcagtcagtgttcttaaccgctgagccatctctccaacccc

SEQ ID NO: 39 - RNA, 159 nt, synthetic construct
  source 1..159: mol_type = other RNA
ttatttattataagtacactgtagctgtcttcagacacaacaaaagagggcgtcagatctcattacaggtggttgagccaccatgtggttgctgggatttgaactcaggaccttcagaacagtcagtgctcttacccactgagccagcgagccagcccc

SEQ ID NO: 40 - RNA, 77 nt, synthetic construct
  source 1..77: mol_type = other RNA
gaactggagttatacggtaacctcgtggtggttgggaaccaccatgtggatggatattgagttccaaacactggtcc

SEQ ID NO: 41 - RNA, 77 nt, synthetic construct
  source 1..77: mol_type = other RNA
gaactggagttatacggtaacctcgtggtggttcccaaccaccatgtggatggatattgagttccaaacactggtcc

SEQ ID NO: 42 - RNA, 77 nt, synthetic construct
  source 1..77: mol_type = other RNA
ggaccggagttatacggtaaccgcgtggtggttgtgaaccaccacgcggatggatattgagttccaaacaccggtcc

SEQ ID NO: 43 - RNA, 77 nt, synthetic construct
  source 1..77: mol_type = other RNA
gaactagagttatacggtaaccacatggtggttgtgaaccaccatgtggatggatattgagttccaaacactagttc

SEQ ID NO: 44 - RNA, 206 nt, synthetic construct
  source 1..206: mol_type = other RNA
ttattttaaatatatgagtatttcacctgcataggcgcacagtacccacagagactagaagagggtagtagatcccctagaactggagttatacggtaacctcgtggtggttgtgagctaccatgtggatggatactgggaatcaaacccaggtcctgtggaaggcaggcaggtgctctcaagcactgagccatctcttcagctcc

SEQ ID NO: 45 - RNA, 206 nt, synthetic construct
  source 1..206: mol_type = other RNA
ttattttaaatatatgagtatttcacctgcataggcgcacagtgctcaaggagatcagaagagggcatcagatctcctgagactggagttatacggtaacctcgtgatggttgtgaactaccatgtggatggatattgagttccaaacacaggtcctgtgcaagagcagcaggtgctcttaagcacggaaccatctctttagctcc

SEQ ID NO: 46 - RNA, 156 nt, synthetic construct
  source 1..156: mol_type = other RNA
gaggctagaagagggtatcagatcccctgagactggagttatacggtaacctcgtggtggttgtgagccaccatgtggatggatactgagaaccaaaccctggtcctgtgcaagagcatcaggtgctcttaagcacggaaccatctcttcagctcc

SEQ ID NO: 47 - RNA, 68 nt, synthetic construct
  source 1..68: mol_type = other RNA
gtcctgtgcaagagcatcgaactcggtgctcttaagcacagaagccaccaagccatctcttcagcccc

SEQ ID NO: 48 - RNA, 110 nt, synthetic construct
  source 1..110: mol_type = other RNA
cagtgctagaggaggtcagaagagggcatcccccagcctcgtggtggttgtgaaccaccatgtggctgtgcaagagcatgctcttaagtgctgagccatctctttagctc

SEQ ID NO: 49 - RNA, 126 nt, synthetic construct
  source 1..126: mol_type = other RNA
gagggcattggatcccccagaactggagttatacggtaacctcgtggtggttgtgaaccaccatgtggatggatattgagttccaaacactggtcctgtgcaagagcatccagtgctcttaagtgc

SEQ ID NO: 50 - RNA, 89 nt, synthetic construct
  source 1..89: mol_type = other RNA
ggatcccccagaactggagttatacggtaacctcgtggtggttgtgaaccaccatgtggatggatattgagttccaaacactggtcctg

SEQ ID NO: 51 - RNA, 72 nt, synthetic construct
  source 1..72: mol_type = other RNA
tgctagaggaggtcagaagagggcattggatgcaaatccagtgctcttaagtgctgagccatctctttagct

SEQ ID NO: 52 - RNA, 94 nt, synthetic construct
  source 1..94: mol_type = other RNA
gagggcattggatcccccagaactggagttatacggtaacgatggatattgagttccaaacactggtcctgtgcaagagcatccagtgctctta

SEQ ID NO: 53 - DNA, 1111 bp, synthetic construct
  source 1..1111: mol_type = other DNA
cagtgctagaggaggtcagaagagggcattggatcccccagaactggagttatacggtaacctcgtggtggttgtgaaccaccatgtggatggatattgagttccaaacactggtcctgtgcaagagcatccagtgctcttaagtgctgagccatctctttagctccagtctcttaaaaaacaaacaaacgaacgaacagcaagggagctgggtatgacaacacatactataattctagtactcaggatgctgaaacaggaggattgcctgactgggagatataaggagaatctgttgtcacccccacccctccccataaaggcagaataaaagaacgtcctataaacaaataaacaaacaacccaataaaacaaaaccaagatctctccaccttttctttgctttttcagactttgtaataaggccctttggagtgcaggatattcggcaggacaagcagagagggagaccatcagttctttctttgatcaagaagactatgttccttagcaaactggtgtgtattatctcttatgcaatgagcctggaaagagggcacagccaccgaggatggtacagcatggatggatggtacgctacagagactcgggagcccaactgtgagtggctgactggcatggtaggttcagggaagaattggcctgtgaagaaaatgttcttgaaaagtgaacaaggtgcaggaggtaggagtgggtcctgggcaaagcagggggtgcatcccagcctcagggaatagcacagcagaggtctgttgatgcatgcgagtgcatgacctgcttgccaatagacgatcaagaatgggcaaagcatcatgggtgatgagtgggagaggggatgagacattcctttctccctgctgagacttccattgaaccgatgagttctgaatagaagatgcccccccacccccccaccagtgtagaatctgaagggaggcatatattaccctatattactctgtgttggcggcgagctatctgacagccaaccttcccatacatttcattgggcatacactaatgacaggaagttccttttgcttgtatgcaagagatggctcacacgatggagaatttaatcttgaagggc

SEQ ID NO: 54 - DNA, 1154 bp, synthetic construct
  source 1..1154: mol_type = other DNA
gatgcggccgccactgtgctggatatctgcagaattcgcccttcagtgctagaggaggtcagaagagggcattggatcccccagaactggagttatacggtaacctcgtggtggttgtgaaccaccatgtggatggatattgagttccaaacactggtcctgtgcaagagcatccagtgctcttaagtgctgagccatctctttagctccagtctcttaaaaaacaaacaaacgaacgaacagcaagggagctgggtatgacaacacatactataattctagtactcaggatgctgaaacaggaggattgcctgactgggagatataaggagaatctgttgtcacccccacccctccccataaaggcagaataaaagaacgtcctataaacaaataaacaaacaacccaataaaacaaaaccaagatctctccaccttttctttgctttttcagactttgtaataaggccctttggagtgcaggatattcggcaggacaagcagagagggagaccatcagttctttctttgatcaagaagactatgttccttagcaaactggtgtgtattatctcttatgcaatgagcctggaaagagggcacagccaccgaggatggtacagcatggatggatggtacgctacagagactcgggagcccaactgtgagtggctgactggcatggtaggttcagggaagaattggcctgtgaagaaaatgttcttgaaaagtgaacaaggtgcaggaggtaggagtgggtcctgggcaaagcagggggtgcatcccagcctcagggaatagcacagcagaggtctgttgatgcatgcgagtgcatgacctgcttgccaatagacgatcaagaatgggcaaagcatcatgggtgatgagtgggagaggggatgagacattcctttctccctgctgagacttccattgaaccgatgagttctgaatagaagatgcccccccacccccccaccagtgtagaatctgaagggaggcatatattaccctatattactctgtgttggcggcgagctatctgacagccaaccttcccatacatttcattgggcatacactaatgacaggaagttccttttgcttgtatgcaagagatggctcacacgatggagaatttaatcttgaagggc

SEQ ID NO: 55 - RNA, 383 nt, synthetic construct
  source 1..383: mol_type = other RNA
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaac

SEQ ID NO: 56 - RNA, 383 nt, synthetic construct
  source 1..383: mol_type = other RNA
gttacgtttggtttttctttgaggtttaggattcgtgctcatggtgcacggtctacgagacctcccggggcactcgcaagcaccctatcaggcagtaccacaaggcctttcgcgacccaacactactcggctagcagtcttgcgggggcacgcccaaatctccaggcattgagcgggtttatccaagaaaggacccggtcgtcctggcaattccggtgtactcaccggttccgcagaccactatggctctcccgggagggggggtcctggaggctgcacgacactcatactaacgccatggctagacgctttctgcgtgaagacagtagttcctcacaggggagtgattcatggtggagtgtcgcccccatcagggggctggc

SEQ ID NO: 57 - RNA, 312 nt, synthetic construct
  source 1..312: mol_type = other RNA
atgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggcc

SEQ ID NO: 58 - RNA, 312 nt, synthetic construct
  source 1..312: mol_type = other RNA
ggccaatccaattcgctttatgataacaatctgtgattgtcaccataagcagccacaataaaataaaaggaaacacggacacccaaagtagtcggttccgccacggacttgcgcgttacgacaggccaatcactggtttgtgaccacctgctccgaggttgggattagccgcattcaggggccggaggattcttatgtagctcaataggctcttcacaccttgttcacaactagcgtcccatggcgttagccataggtaggccgccaacgcagcctggaccaccgtcaccggtgagggatgtccagactcat

SEQ ID NO: 59 - RNA, 576 nt, synthetic construct
  source 1..576: mol_type = other RNA
ccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataa

SEQ ID NO: 60 - RNA, 576 nt, synthetic construct
  source 1..576: mol_type = other RNA
ttatcatcgtgtttttcaaaggaaaaccacgtccccgtggttcggggggcctagacgttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggggg

SEQ ID NO: 61 - RNA, 192 nt, synthetic construct
  source 1..192: mol_type = other RNA
aaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacct

SEQ ID NO: 62 - RNA, 192 nt, synthetic construct
  source 1..192: mol_type = other RNA
aggtaaatttcttaggtttttcgactacctaatctgaaaaaccgcagagagggcttcctggatattgtggggctgccactaggcatcctggaacgtaaagctaaatagctaacctaaatacaaaaatagcactacttgtaatttattaacctctcaaaattgtatttacaagcaagatcacatttttgcttt

SEQ ID NO: 63 - RNA, 231 nt, synthetic construct
  source 1..231: mol_type = other RNA
cagagatccaggggaggcgcctgtgaggcccggacctgccccggggcgaagggtatgtggcgagacagagccctgcacccctaattcccggtggaaaactcctgttgccgtttccctccaccggcctggagtctcccagtcttgtcccggcagtgccgccctccccactaagacctaggcgcaaaggcttggctcatggttgacagctcagagagagaaagatctgaggga

SEQ ID NO: 64 - RNA, 231 nt, synthetic construct
  source 1..231: mol_type = other RNA
tccctcagatctttctctctctgagctgtcaaccatgagccaagcctttgcgcctaggtcttagtggggagggcggcactgccgggacaagactgggagactccaggccggtggagggaaacggcaacaggagttttccaccgggaattaggggtgcagggctctgtctcgccacatacccttcgccccggggcaggtccgggcctcacaggcgcctcccctggatctctg

SEQ ID NO: 65 - RNA, 460 nt, synthetic construct
  source 1..460: mol_type = other RNA
acttttggtgggcatttaaaaatgtgtgtgtatgtgtatatatgtatgtgtatgtatgtgtatatatgtatatgtatgtatgtatcgcgtgtatgtgtgtatgtatgcatgtgtatgtatgtatatgcatgtatgtgtatgtgtatatatgtatgtgtgtgtatgtatatgtgtgtgtatgtgtatgtgtgtgtgtatgtgtgtgtgtatgtatgtatgtatgtatatgtattatacacatatacacatattggtttttttaatcatttgagagttagttgaagataaaaacccatcacccctaaatgtattccaaagaataagaacattgttttatacatagcacacttaacaaaatcaagaaatttaacattaatacagtactgttacctaatccgtagtcgattttcaaattttgtcagttgttccaataatgtcctttatatattccccgcccagc

SEQ ID NO: 66 - RNA, 460 nt, synthetic construct
  source 1..460: mol_type = other RNA
gctgggcggggaatatataaaggacattattggaacaactgacaaaatttgaaaatcgactacggattaggtaacagtactgtattaatgttaaatttcttgattttgttaagtgtgctatgtataaaacaatgttcttattctttggaatacatttaggggtgatgggtttttatcttcaactaactctcaaatgattaaaaaaaccaatatgtgtatatgtgtataatacatatacatacatacatacatacacacacacatacacacacacatacacatacacacacatatacatacacacacatacatatatacacatacacatacatgcatatacatacatacacatgcatacatacacacatacacgcgatacatacatacatatacatatatacacatacatacacatacatatatacacatacacacacatttttaaatgcccaccaaaagt

SEQ ID NO: 67 - RNA, 395 nt, synthetic construct
  source 1..395: mol_type = other RNA
aattccagcgagaggcagagggagcgagcgggcggccggctagggtggaagagccgggcgagcagagctgcgctgcgggcgtcctgggaagggagatccggagcgaatagggggcttcgcctctggcccagccctcccgcttgatcccccaggccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcga

SEQ ID NO: 68 - RNA, 395 nt, synthetic construct
  source 1..395: mol_type = other RNA
tcgcgggaggctgctggttttccactacccgaaaaaaatccagcgtctaagcagctgcaaggagagcctttcagagaagcgggtcctggcagcggcggggaagtgtccccaaatgggcagaatagcctccccgcgtcgggagagtcgcgtccttgctcgggtgttgtaagttccagtgcaaagtgcccgcccgctgctatgggcaaagtttcgtggatgcggcaagggttgcggaccgctggcctgggggatcaagcgggagggctgggccagaggcgaagccccctattcgctccggatctcccttcccaggacgcccgcagcgcagctctgctcgcccggctcttccaccctagccggccgcccgctcgctccctctgcctctcgctggaatt

SEQ ID NO: 69 - RNA, 48 nt, synthetic construct
  source 1..48: mol_type = other RNA
gggcactttgcactggaacttacaacacccgagcaaggacgcgactct

SEQ ID NO: 70 - RNA, 48 nt, synthetic construct
  source 1..48: mol_type = other RNA
agagtcgcgtccttgctcgggtgttgtaagttccagtgcaaagtgccc

SEQ ID NO: 71 - RNA, 71 nt, synthetic construct
  source 1..71: mol_type = other RNA
gtactgacatcgtagatggaaatcataaactgactcttggtttgatttggaatataatcctccactggcag

SEQ ID NO: 72 - RNA, 71 nt, synthetic construct
  source 1..71: mol_type = other RNA
ctgccagtggaggattatattccaaatcaaaccaagagtcagtttatgatttccatctacgatgtcagtac

SEQ ID NO: 73 - DNA, 1786 bp, synthetic construct
  source 1..1786: mol_type = other DNA
ctctgatcagcggcgggtggccttcgggtcatgatctccgccgtgtctagtccgtggctcacgcagctctcgcacttctgcgacgttgcagccttcgcagccagcagtctgagcggcctgggatccccgtcgcctggcgccgacccgttcggccctcgcgagccgccgccaccgcgctacgatccgtgcgctgcagtccccggtgccccgggcccgccgccgccgcgcgcctatcctttcgcgcccgcccccggggcggctggcagctcggcggcggagtccgagggtccgggggctagccgcgcggctgcggtcaaggctccggtgaagaagaacccgaaggtggccagcgtgagcgtgcagctggagatgaaggcgctgtgggacgagttcaatcagctgggcaccgagatgatcgtcaccaaggcaggcagacgaatgttccccacgttccaagtgaagctttttggaatggatcccatggccgactacatgctgctcatggactttgtgcccgtagatgacaagcgctaccggtatgctttccatagctcctcctggctggtggccggcaaggcagatcctgctacacctggccgagtacactaccacccggactcgccggctaagggcgcacagtggatgaaacagattgtgtctttcgacaagctgaaactgaccaataacctgctggatgacaatggccatattattctcaactccatgcacagatatcagccccgattccatgttgtctatgtggaccctcgaaaagacagtgagaaatatgcagaggagaacttcaaaacttttgtgtttgaggagacacgcttcactgcagtcactgcctaccagaatcaccggatcacgcagcttaagattgccagcaaccccttcgccaaaggcttccgggattgcgacccggaggactggccccggaaccaccggcccggagcgctgccgctcgtgagtgcctttgctcgctctcggaatcccgtggcttcccccacgcagcccaatggctcagacaaagacgctgcagaagcccggcgcgagttcgaccgtgactccggacccgcagcgctcggcgacgctacgcacccgccgcagctgctggcgcgcgtgctgagccccgcactgcccgggcctggcggcctcgtcccgctacccggcggatccggaggccgccacagtcccccgcacgccgatctgcgcctggaggcgccgggcgcgtccgagccgctgcaccaccatccctacaagtacccggccgccgcctacgaccactacctcggggccaagagccggccggcgccctacccgctgccaggcctgcgcggccacggctaccacccgcacgcgcacccgcacgcgcacccgcaccatcaccaccaccccgcggtgaacccggccgccgccgccgctgctgccgcagcagccaacgtgtactcgtcggcggccgcgccgcccggtgcctacgactactgccccagatagtgcgcccgcgcgccgaccccgagggccatccaaggacgcgctccccatctggggagccatgcgggtttcccgcccgccagtgccaaagctcccgtccaggcggaaggaagtggtatttattgttctccgcgagacctcgtcgcctccggcccggccggcaattgcagtgtagacgaccgagagagccccgcctgcgggcggtgtagatacgtgtagatatgtgtagatactgtagatactgcaccggcgccgatttcataaacggttttgcctcttttggaaattgccg

SEQ ID NO: 74 - DNA, 1782 bp, synthetic construct
  source 1..1782: mol_type = other DNA
ctctgatcagcggcgggtggccttcgggtcatgatctccgccgtgtctagtccgtggctcacgcagctctcgcacttctgcgacgttgcagccttcgcagccagcagtctgagcggcctgggatccccgtcgcctggcgccgacccgttcggccctcgcgagccgccgccaccgcgctacgatccgtgcgctgcagtccccggtgccccgggcccgccgccgccgcgcgcctatcctttcgcgcccgcccccggggcggctggcagctcggcggcggagtccgagggtccgggggctagccgcgcggctgcggtcaaggctccggtgaagaagaacccgaaggtggccagcgtgagcgtgcagctggagatgaaggcgctgtgggacgagttcaatcagctgggcaccgagatgatcgtcaccaaggcagacgaatgttccccacgttccaagtgaagctttttggaatggatcccatggccgactacatgctgctcatggactttgtgcccgtagatgacaagcgctaccggtatgctttccatagctcctcctggctggtggccggcaaggcagatcctgctacacctggccgagtacactaccacccggactcgccggctaagggcgcacagtggatgaaacagattgtgtctttcgacaagctgaaactgaccaataacctgctggatgacaatggccatattattctcaactccatgcacagatatcagccccgattccatgttgtctatgtggaccctcgaaaagacagtgagaaatatgcagaggagaacttcaaaacttttgtgtttgaggagacacgcttcactgcagtcactgcctaccagaatcaccggatcacgcagcttaagattgccagcaaccccttcgccaaaggcttccgggattgcgacccggaggactggccccggaaccaccggcccggagcgctgccgctcgtgagtgcctttgctcgctctcggaatcccgtggcttcccccacgcagcccaatggctcagacaaagacgctgcagaagcccggcgcgagttcgaccgtgactccggacccgcagcgctcggcgacgctacgcacccgccgcagctgctggcgcgcgtgctgagccccgcactgcccgggcctggcggcctcgtcccgctacccggcggatccggaggccgccacagtcccccgcacgccgatctgcgcctggaggcgccgggcgcgtccgagccgctgcaccaccatccctacaagtacccggccgccgcctacgaccactacctcggggccaagagccggccggcgccctacccgctgccaggcctgcgcggccacggctaccacccgcacgcgcacccgcacgcgcacccgcaccatcaccaccaccccgcggtgaacccggccgccgccgccgctgctgccgcagcagccaacgtgtactcgtcggcggccgcgccgcccggtgcctacgactactgccccagatagtgcgcccgcgcgccgaccccgagggccatccaaggacgcgctccccatctggggagccatgcgggtttcccgcccgccagtgccaaagctcccgtccaggcggaaggaagtggtatttattgttctccgcgagacctcgtcgcctccggcccggccggcaattgcagtgtagacgaccgagagagccccgcctgcgggcggtgtagatacgtgtagatatgtgtagatactgtagatactgcaccggcgccgatttcataaacggttttgcctcttttggaaattgccg

SEQ ID NO: 75 - DNA, 1858 bp, synthetic construct
  source 1..1858: mol_type = other DNA
ccggtagggggagcgaggcggaagggagcggcggccggtgcagccgaggcctcggagggcaccgcccaccggggccccaggccctcggaccgggcgaaacttcgccggctaccaggatccccagccgggatgcacttcagcacagtcaccagggacatggaagccttcgcagccagcagtctgagcggcctgggatccccgtcgcctggcgccgacccgttcggccctcgcgagccgccgccaccgcgctacgatccgtgcgctgcagtccccggtgccccgggcccgccgccgccgcgcgcctatcctttcgcgcccgcccccggggcggctggcagctcggcggcggagtccgagggtccgggggctagccgcgcggctgcggtcaaggctccggtgaagaagaacccgaaggtggccagcgtgagcgtgcagctggagatgaaggcgctgtgggacgagttcaatcagctgggcaccgagatgatcgtcaccaaggcaggcagacgaatgttccccacgttccaagtgaagctttttggaatggatcccatggccgactacatgctgctcatggactttgtgcccgtagatgacaagcgctaccggtatgctttccatagctcctcctggctggtggccggcaaggcagatcctgctacacctggccgagtacactaccacccggactcgccggctaagggcgcacagtggatgaaacagattgtgtctttcgacaagctgaaactgaccaataacctgctggatgacaatggccatattattctcaactccatgcacagatatcagccccgattccatgttgtctatgtggaccctcgaaaagacagtgagaaatatgcagaggagaacttcaaaacttttgtgtttgaggagacacgcttcactgcagtcactgcctaccagaatcaccggatcacgcagcttaagattgccagcaaccccttcgccaaaggcttccgggattgcgacccggaggactggccccggaaccaccggcccggagcgctgccgctcgtgagtgcctttgctcgctctcggaatcccgtggcttcccccacgcagcccaatggctcagacaaagacgctgcagaagcccggcgcgagttcgaccgtgactccggacccgcagcgctcggcgacgctacgcacccgccgcagctgctggcgcgcgtgctgagccccgcactgcccgggcctggcggcctcgtcccgctacccggcggatccggaggccgccacagtcccccgcacgccgatctgcgcctggaggcgccgggcgcgtccgagccgctgcaccaccatccctacaagtacccggccgccgcctacgaccactacctcggggccaagagccggccggcgccctacccgctgccaggcctgcgcggccacggctaccacccgcacgcgcacccgcacgcgcacccgcaccatcaccaccaccccgcggtgaacccggccgccgccgccgctgctgccgcagcagccaacgtgtactcgtcggcggccgcgccgcccggtgcctacgactactgccccagatagtgcgcccgcgcgccgaccccgagggccatccaaggacgcgctccccatctggggagccatgcgggtttcccgcccgccagtgccaaagctcccgtccaggcggaaggaagtggtatttattgttctccgcgagacctcgtcgcctccggcccggccggcaattgcagtgtagacgaccgagagagccccgcctgcgggcggtgtagatacgtgtagatatgtgtagatactgtagatactgcaccggcgccgatttcataaacggttttgcctcttttggaaattgccg

SEQ ID NO: 76 - DNA, 4226 bp, synthetic construct
  source 1..4226: mol_type = other DNA
ggtcggtgagggtcgaccggctgtggtcgggctgcgggcggctcgggcaggtcgcgggcgccacaggtggaagaagaaaggtgccactttggcatgaagatagactcacttagacgtcaatcttttaagctgagtgcattgtgatttccaataattgaggcagtggttctaaaagctgtctacattaatgaaaagagcaatgtggccagcttgactaagccgccagtgtgtacagcgcgggcaggacgacaccgggtctcgacggacttgtgcatgttagcagtatagatttatgtaaggtggtttaaaactctggtcttttaaagtagtcttaactgctcataatatggagacatatgagagtccctctcctctcccgcgtgagcccgcaggagaagcgatgatggagaaccgagcttgccccttccaagtgctgccccatgaacagtctccaccacctcccctgcaaacgtccagtgatgcagaggtaatggacgttggctctggtggtgatggacagtccgaacctcctgccgacgacccattcaacttctacggagcttctcttctctccaaaggatccttctctaagggccgcctcctcatagacccgaactgtagtggccacagcccgcgcactgcccggcacgcacctgcggtccggaagttctcccctgaccttaagttgcttaaggatgtaaagattagcgtgagctttactgagagctgcaggagtaaggacaggaaggtgctgtacacaggagtagaacgcagcactcggcctgagtgtggccagctccttagtcctgtcagtggggacgtgcatgcttgtccctttggcgggagtgttggtaatggggtaggcctagggggtgagagtgcagataagaaggatgaggaaaatgagctggatcaggaaaagagagtggagtatgcagtgctcgatgagttagaagattttactgacaatttggagctagatgaagaaggaacaggcgggttcacggctaaagcaatcgttcaaagagacagagtggatgaagaggccttgaatttctcctatgaggatgactttgacaatgatgtggacgctttactagaagaaggtctctgtgctcccaagaagaggcgaatggaggaaaaatatggcggagacagtgatcatccatctgatggagagacaagtgtacagccaatgatgaccaagattaaaacagtgctcaaaagtcgtggccgtccacctacagagccattgcctgatggatggatcatgacttttcataattctggagtccctgtatacctgcacagagagtctcgagtggtcacttggtccagaccctacttcttgggaacaggaagcatacggaaacatgatcctcctctaagcagtatcccctgcctacattataagaaaatgaaggacaatgaggaacgagaacaaaactgtgatcttgcccccagtggagaggtgtcacctgtcaagcccttgggtcggtctgcagagttggatttccctctggaagagcctgactccatgggtggagactcagggtccatggatgagaaggacccattgggggctgaggcagccgctggagccctgggacaagtgaaggctaaagttgaggtgtgcaaagatgaatcagttgatctggaggaatttcgtaattaccttgagaagcgttttgactttgaacaagtaactgtgaaaaaattcaggacttgggctgagcggcgtcagttcaaccgtgagatgaagcggaagcaggccgagtcagagaggcccatcctgccagccaaccagaagctgatcactctatctgtacaagatgcacccacaaagaaagagtttgtcatcaatcccaatgggaagtctgaggtttgcatcctgcacgaatacatgcagcgtgtcctcaaggtccgccctgtttataatttctttgaatgtgagaatccaagtgagccttttggtgcctccgtgaccattgatggtgtgacttacggatctggaactgcaagcagcaaaaaacttgcgaagaataaagctgcccgagccaccctggaaattctcatccctgactttgttaaacagacctctgaggagaagcctaaagacagtgaagaactggagtattttaaccacatcagtattgaggattcacgagtctatgagctgacaagcaaggctgggctgttgtctccatatcagatcctccatgagtgccttaaaagaaaccatggaatgggtgacacatccatcaagtttgaagtggttcctgggaaaaaccagaagagtgaatatgttatggcatgcggcaaacacacagtgcgcgggtggtgtaagaataaacgagttgggaaacaattagcatctcagaaaatccttcagctactgcacccacatgtcaagaactggggttccttactacgcatgtatggtcgtgagagcagcaaaatggtcaagcaggagacctctgacaagagtgtgatagagctacagcagtatgccaagaagaacaggcccaaccttcacatcctgagcaagctacaagaagagatgaagaggctggctgcagagcgggaggagactcggaagaaacccaagatgtcaattgtagcatctgcccagcctggtggtgagcccttgtgcacagtcgatgtatgaggtaggcagcatgggccaacagtgctacccaggagagaccatcagccacacatcatcactctgcagctccaggcctccaacctaagttccttccctgtggcagggtctgggctctggctctggcacatggggacagctgtgactacacagtacacatgcaaagaagcagttctggacaagctgctctcaatgtgactggatatacttaaggatcagtcataagttaactgcacaagtggaagctgatagacagctgtagatcctgcttgtatgtatctgctgcggaccgtttttatgaaggttttcattaattttagtacactatatgcactgacaagaagtaatacttcgttgatagatgagatgaccaggttttatagctttggctggcctatgatgccaggtagcccctttgctatggttctttctggtggagccactgagacttggtcagaagatgcaggtatcctgtcctgtagaattacctgctttgtgtgttttgcattttcttctgaaaaagttataacagaaaggaatatttcaggctattttggcttaaaataaaacaaaattttagcaccagaaatactcttccgagattgcagtgaagatactgagtgttagtccgattgatcctttttcttatgaagtcttaatgtcctaacagccgtcttctggatactgaagcttccaccttcgataaacttagcaacaattttttacagagatttttgcacaaccaagcccatctgttcatcaatttttaaagcttttatgatgttttaaaatttggaaagaaacttttacagtaagaatgaaagatgcatttcaacagatgaatatgcataaatagggaactggccttcaatgtagcccatcactgaccaagagctgcctggcacattgtacacgataagtagttatggtgttttttgatctcaaggatagctgctctgtgcagccaggcctgattgcaggtttttgtctacctatgctcattggatagcccctgtaaatgggtattgagtagaggggtattcagagccctattacctgttcagaggggctacactctacatcctcagtgtctctaaccatgtacctgaagatacatgggtgacatgtgctgttagaaagataagatggcaactgtgggcctcccaacagcatcttcctcctccttaatcatgtgtgtgacagcagttaatgtttatgttaatcttggcaaaaagaacccttaattcaagttggtagacttggacacttgattcttttctgcctcccattcagttgtgttcagtcttttgactaaccattgcccttaaaacatacttaacattgaaaaagcatatggtaatggtggtatctctgcagcctcaccgacccaggcactgtcaacaggtgctgccacacattcctggtctccagtgccctccctgtctcttacaggtgggtcttatatgtacagtcatgctgcaagggtggggagcctgtgctgatgtgcttctgtgtgttggccaaggctgtagggtgtaccctgatggtctgaacacataaa

SEQ ID NO: 77 - DNA, 1400 bp, synthetic construct
  source 1..1400: mol_type = other DNA
gcagccctgctagaagcaggtcccctcctaatcagaaccctggcctgtgtgggcacgcccacgcggacacgttccaggacagccccgccaccgcgaccccctgtggacactcgcacaggcaccctctccgcgtgcaccgcccacgaccccaccctacctcccgctgcactcgccgcgccccttgtcccgcgtgtcagagcccgtgtccgcggccggaagcgcctgctgggtccacgcagcgccgccaccatcgccgccaccatcgccgccatggtccggggacctcaggcggtcagggctgcccttcgcactgcctgagcgtccgcgcctcggggtcagacctgtgcgcggccttgacgaggggatgagagagtcctaccacagtgaaactcaaagttacagacattctggcccataaatgctgttggctgctgtctcattgggtctcctgttgctggccttcctcctgctcctgcgacacctaggctggggcttggtggctattggttggtttgagttcgtgcagcagccggtccacaacctgctcatgggtggcacaaaggagcagcgcatcctgcgccatgtgcagcaacacgcaaagcctggagacccccagagcgtcctggaggccattgatacctactgctcagagaaggagtgggccatgaacgtgggtgacgcaaaaggccaaatcatggatgcagtgattcgggagtacaggccctcgctggtgctggagctaggagcttattgtggctactcagccgtgcgaatggcccgcctgctgccacctggagccaggcttctcaccatggagattaaccctgactacgctgccatcacccagcaaatgctggacttcgcaggcctacaggacaaagtttccatcctcatcggggcatcccaggaccttatcccccagctgaagaagaagtacgatgtggacacattagacatggtctttcttgaccactggaaagaccgctaccttccagacacacttctcctggaggaatgtggcctgctgcgcaaggggacggtgctcctagctgacaatgtcattgtcccgggaacccctgacttcctggcgtatgtgagggggagcagcagcttcgagtgcacacactacagctcatacctggagtacatgaaagtggtggacggcttggagaaggcagtctaccagggtccaggcagcagccccgtgaagtcctgaccactcagcctgatgagcttccgtcccagctcccttctgcacgatgacacacactcactctgaccccctctatgcttctggggcctttcctcagggcctgtggctccagattgtcatacactggcacattaaaggtagtgagctcaccatgcaaaccactacaatacccctggaaaacacctgt

SEQ ID NO: 78 - DNA, 4553 bp, synthetic construct
  source 1..4553: mol_type = other DNA
gcggtgttttcctgctctctgctccgcctggcctatgtgtttacatctatgaaaacgccccgctcttctaataaatgcgcccgtctggccccctatccttcaccagcctagttcttttatgcatgcaatcgagctcagggcccgctgccgtctgccgctagatgcatgctggcgcccgcccggctggcgctcgcgagatgcccaatagcgcgaattgatggatggcggaagctagttactaagctggactcggagccgcggaggtagaactagagatctgcacaccaccctggttctgcatcgccgggcgggatctcaaggctttgccagcctgctcacaccgaccctcatcggcgaccacatcagtggcagcggagtatccccgtcctcccccccctgtctaccgaagaaccgaggcggcccggtccgcgtagggagcggtttcccgccggggcgatttggcaggtgcgcgccgtgacttccggcgttgcccgggagccgccggaggaggagcggcgcaggggatgcggctgtggcggcggcggcggcggccgagcgcaggaggcggctgtggcggcggctgggggcacgggccggcgatggcgcggcggcgctgagggcaagggtgggcggcggccggagggcgggcggcgcgggaggaagctgcggcagctccatggcccaggcgtgctgagggacacggctctggcttcagcccggcagcggccgaacaatgaagctcttgaagccaacctgggtcaaccacaatgggaagccaattttttcagttgatattcaccctgatgggaccaagtttgcaactggaggacaagggcaggattctgggaaggttgtgatctggaatatgtctccagtcctccaggaggatgacgagaaggatgaaaatattcccaagatgctttgccagatggacaatcacttagcatgtgtgaactgtgtgcggtggtcaaacagtgggatgtatttagcttctgggggagatgacaaactgattatggtgtggaagcgggctacgtacattgggcccagcactgtgtttggttccagtggtaagcttgccaatgtggagcaatggcggtgtgtctccatcctccggagtcactcaggcgatgtgatggatgtagcatggtctccccacgacgcctggctggcctcatgcagcgtggataacactgttgtcatttggaatgccgtgaagttcccagaaattcttgcaactctgagaggtcattctggcttagtaaaaggtttgacttgggatcccgttggtaaatatattgcctctcaagctgatgatcgaagtttgaaggtatggaggacgctggactggcagctagagactagcatcaccaagccttttgatgagtgtggaggaacgacgcatgttctccggcttagttggtcacctgatggccattacctggtatctgcccatgccatgaataattctggccccactgctcagatcatcgaaagagagggctggaagaccaacatggactttgtgggtcaccggaaagctgtgactgttgtgaaattcaacccaaaaatcttcaagaagaagcagaagaatgggagctctacaaagcccagctgcccatactgctgctgtgctgttggcagcaaggaccgctcactctctgtctggctcacatgtttgaaacggcctctggttgtcatccatgaactgtttgacaagtccatcatggatatttcctggactctgaatgggttgggtatcctggtgtgctccatggacggctctgtggcgttccttgatttctctcaggatgaactcggagaccccctgagtgaggaggaaaagagccgaattcaccagtctacctatggcaagagcctggcaataatgactgaggcccagctttccacagctgttattgagaaccctgagatgctcaagtaccagcggagacagcaacagcagcagctggatcagaagaatgccactactagggagacaagctcagcatcctcagtcacgggtgtggtcaatggggaaagtctagaagatatcagaaagaatcttttgaagaaacaagtggaaactcggacagcagatggtcggaggagaatcacgcctctttgcatagcacagctggacactggggacttctccacggcattcttcaacagcatcccactctccagctccctagcaggcaccatgctctcctctcctagtggtcagcagctactaccactggactccagtaccccctccttcggcgcctcaaagccttgcacagaaccagtggcagccaccagtgccaggcctacaggcgaatctgtcagtaaggacagtatgaatgctacctctactcctgctgcatcgtcaccctctgtgttaacaaccccatccaagattgaacccatgaaagcatttgattcccggttcacagaacggtccaaagccacaccaggtgctccttccttgaccagtgtgattccaacagctgttgaaaggttgaaagagcagaacctcgtcaaggagctgaggtcccgggaactggagagcagcagtgacagcgatgagaaggtccacctagccaagccctcttcactgtccaagcgcaaacttgagcttgaggtagagacggtggaaaagaagaagaaaggtcgccctaggaaggattcacgtcttttacccatgtctctgtccgtccagtctccagctgccctgtctacagagaaggaggccatgtgtctgtctgcaccagcacttgcactgaagctgccaattccaggcccacagagagcgttcaccctccaggtgagctctgacccctccatgtacattgaggtggagaatgaagtgaccaccgttggggggataaggctgagtcgcctgaagtgcaaccgtgaagggaaggagtgggagacagtgctcagcagtcgcgtcctcactgctgccggcagctgtgatgtggtatgtgttgcctgtgaaaagaggatgctgtcggtgttctctacctgtggtcgccgtctcctccctcccatcctccttccatctccaatctctactttgcactgcacgggcccctacgtcatggcactcaccgctgcagccacactgtctgtctgggatgttcacagacaggtggttgtggtgaaagaagaatctctacactccatcttgtcaggaagtgatatgacggtgtcacagatcttgctaacacagcatggaatcccagtgatgaatttgtctgatgggaaagcatactgctttaatccatccctctccacatggaacctggtttctgacaagcaagattcattggcccagtgtgcagacttcaggaacagcctgccgtcccaggatgccatgctttgttcaggaccgttagccataattcagggccgcacctccaactctggaaggcaagctgcccggctcttctccgtgcctcatgtggtacagcaggagaccacactggcctacctagagaaccaggttgctgcggcacttaccctgcagtcaagccacgagtatcgtcactggctcctcctttatgctcggtaccttgtgaatgaagggtttgaataccgcctccgtgaaatatgcaaggacttgctgggtccagttcactgctccactggaagtcagtgggagtcaacagtagtgggtctgcggaaaagggagctgctgaaggaactgttgccagtcattgggcagaatcttcgattccagcgcctctttactgagtgccaggaacaactggacatcctgagagacaagtagtctgtcctggcttgcctcagctgctgcaagggcaggaccacactattgccactggcaggttgcccagggctagccccacctgtcagggcggtagtaggaagagagaccttggcagaagatgtgttgtcctgcaccagcactagcccagttccctgggtggacatggactatatcccaagcctccatgactccaaaagggggcagcttgtccctccatgtaggcccagctgtggacttggggctgggactggggctctgccttgctgccagcagggatctcttgagcctgtctccagcttgaccccaaggtagacactggcatctgccactgcaggtgctgtgctgcttcagctgtaacgaatgagccatttgtgagagcaggagccggggagtgttagggatgatgcagtcggcagacggacccgaagcctgtgtgaatgctaagctgttctgacacatggactatttttgtactagaatttgctaacttggaatatggagtttctgttggttgatcaggatatccctataagttacttggacattggtcacttgtaggaaatttaaactctaattatgacagctacactgaaaaaaaaataattgtactgaaattaacttgtctatcttcatttggtttaatttttaaatgtttgtaaaaagagacactttgtgtggggggtggggtgggacagaggggagggcaatttcttttctaagtgtaaaataaatgaacatgcattggataccaaaaaaaaaaaaaaaaaaaaaa

SEQ ID NO: 79 - DNA, 2283 bp, synthetic construct
  source 1..2283: mol_type = other DNA
ttaagcctcctgagcgcgtgtatcggctccgccatggctctcaagcgcgtcttcttgctgcggtcggtggcaccacgcgtcgctgccctctcaaccaaaccgcaagcccaggaacagcctcccgcgagccctgaggctcttcggggatgtggggcggccaaggctgtgcggccgcctgtgccagccgtggacttcaccaacacgcaggaggcgtatcgcagccggcggagttgggagttggtgcgcaacctgctagtgctgcggctgtgtgcgtcgccggtgctgctagcgcaccacgagcagttgttccaagttgccaggaagcttctggggcaaaggatgttcgagagattgatgaagatgaccttctatggccattttgtggctggcgaggaccaggagtctatcaggcctctgatccggcacaacaaagcctttggtgttggctttatcctggactatggagtggaggaagatctgagccctgaggaggcggagcgcaaagagatggagtcatgcacttctgaagcagagagagatggcagtggagcaaataagagggagaagcagtatcaggtgcaccccgcctttggagaccgcagagatggtgtcatcagtgcccgcacctacttctatgccaatgaagccaagtgtgacaactacatggagaacttactgcagtgcatcaaggcctcaggtggagccagtgatggtggtttctcagccattaagctcactgcactggggagaccacagtttctgctgcagttctcagacgtgctgaccaggtggagacggttcttccatcaaatggctgcagagcagggacaggctgggcgtgctgctgtagacacaaagctggaggtggcggtgctccaggacagcatcgcaaagatgggcatcgcatccagggctgagattgaagggtggttcacgccagagacgctgggagtgtctggcaccgtggacttgctggactggaacagcctcattgacagcaggacccggctctccaggcacttggtggtccccaatgtgcagactggccagctggagcccctgctgtcacggttcactgaggaggaagagcagcagatgaaaaggatgctgcagaggatggatgtactggccaagaaagcaaaagaggcaggtgtgcgcctgatgattgatgctgagcagagctacttccaaccagccatcagccgcctgaccctggagatgcagcgcaggttcaatgtggataagccgttcatcttcaacacattccagtgctacctcaaggatgcctatgacaatgtgaccttggatatggaactggctcgccgtgagggctggtgttttggggccaagctggtacgtggtgcatacatggcccaagagcgtgtcagggcagcagagatcggttatgaggaccccatcaaccctacatatgaagccaccaatgctatgtaccacaggtgccttaactatgttctggaggagctgaagcacagcaccaaggcagaggtgatggtggcttcccacaacgaggacaccgtgcacttcacgttgtgcaggatgaaggagataggcctgcatcctgctgatggtcaggtgtgcttcggacagctgctggggatgtgtgaccaaatcagcttcccactaggccaggcaggctttcctgtgtacaagtatgtgccctatggccctgtgatggaggtactcccttacctgtcccgccgtgccctggagaacagcagcatcatgaagggtgctcagcgagagaggcagctgctatggcaggagctccgcaggcggctgcgcactggcagcctcttccaccatccggcctagtcaccgcaggagccttgcccacccgctcgtactccactcaaccccttacctctggggcttcaggcggggcacagcttgggattgggctggggttccttagcccaacctgcccagacacagttcacctttttatgcccaaggctttttatgcccaaggcgggatttcatcagtggacagctcctgaggaacagtgcccaagatggtcgtctggtcacagaggctgccctctgggacttcctgtaccccaaggaacagacactcaggagtggggtcagctagagcccctgggagctgccccactaatctgagtaagcactgaccacctctgcaggttacagagccctagtccaggattaaccttctgccagggtctaacccatcttccctgcactgggcagaggacagactaggaagcctgtttagtcaataaatcatcctgtaacagagtc

SEQ ID NO: 80 - DNA, 4887 bp, synthetic construct
  source 1..4887: mol_type = other DNA
gggatttcctccactgccgctgcggcggatcctgcgccgcgtccagcccgcgcgcccgaccccggcccgacccggccggccccgcccgcccggcccggggagggatgcggcggcgcggcgcccaggatgccccgcagccccgggacgcgcctcaaacccgccaagtacatcccggtggccacggccgccgcgctgttggttggttccagcacactcttcttcgtattcacgtgcccatggttgacaagagctgtgtctccagctattcctgtctacaatggcatcctcttcctctttgtcctggccaacttcagtatggctaccttcatggaccctggagtcttcccccgagcggacgaggacgaggacaaggaggatgacttccgggccccactgtacaagaatgtggatgtgcggggcatccaggtccgcatgaagtggtgtgcaacgtgccacttttaccgtccaccgcgctgctcacactgcagtgtctgtgacaactgtgtggaggactttgaccatcactgcccctgggtcaacaactgcattggacgccgcaactaccgttacttcttcctgttcctgctgtcactcagcgcgcacatggtgggggtggtggccttcggcctgctctatgtgctcaatcactcggaggggctgggagccgcccacaccaccatcaccatggctgtcatgtgtgtggctggccttttcttcattcctgtcatcggcctcactggctttcacgtggtactggtcacgcggggccgcaccaccaatgagcaggtgactgggaagttccgcgggggtgtgaatcccttcacccgaggctgctatgggaacgtggagcacgtgttatgcagtcccctggcgccccggtatgtggtggaaccccccaggatgccgctctcagtgagcctcaagccacccttcctgagacctgagctcctggaacgagctgtgcccctcaaggtcaaacttagcgacaatgggctgaaagctggccgcagcaagtccaagggcagtctagaccagctggatgagaaacctctggacctgggacctccactgccccccaagatagaggctggtacctttggaagagatctgaagaccccaagacctggcagtgctgagagtgccctatcagtacagaggaccagccccccaacacctgccatgtataagttccggccagccttctccactggtcccaagacacccttttgtggacctaatgagcaggtcccaggtcctgactcccttactctggcagatgacagcacccacagtctagactttgtgtctgagcccagcctggatctcccagaccacgggccgggtggtctgcgtcctccctacccgccctccccacccctcaacaccactgatgccttctcaggtgccttgcgctccctgagtctcaaggctgccagtcggaggggtggggaccacatgaccttacagccactgcgctctgaaggtgggccccctacacctcaccgtagtctctttgctcctcatgcactgcccaatcgaaatggcagcctgtcatatgacagcctacttaaccctggctcacccagtggccacgcatgccccacacacccctctgttggtatagccagctaccattcaccctacctgcaccctgggccatcagatccaccgcggcccccaccccgcagcttcagccctgtgctgggtccccggcctagggaaccctctcctgtgcgctatgacaacctgtctcggaccatcatggcctctatccaggagcgcaaggacagggaagagcgtgaacggctgctgcgctcccagactgactcactctttggcgactctggtgtctatgatacacccagctcctacagcctgcaacaggccagtgtgttaacggaaggcccccgtggctctgtcctgcgttacggctccagggatgacctcgtggccgggcctggctttggtggtgcccgcaatcctgccctgcagacgtcattgtcctcgctgtccagctccatgagtcgggcacctcggacatcttcctcctccctgcaggctgaccaggccaacaacaatgccccaggaccccggcctggcagtggttcacataggtcacctgcccgtcagggcctgccttccccaccaggcaccccccgatcgccctcctacacgggctccaaggccgtcgccttcatccacacagacctcccggaccggcagccctcactggctatgcagagggatcaccctcagctgaagacccccccaagtaagcttaacgggcagtccccaggcatggcccgtctggggcctgctgccagccccatggggcccaacgccagccctgcccggcacacgctggttaagaaggtgtccggcgtgggtgggactacgtatgaaatctcggtgtgagaactgaccaccactcatctgctgtgatgccacggggaccaggaccccccgcagtagctccccaaccctatcaagttctctgccccagggaggaggctccccaagcctgctgtggacacatcaacaggaaagacagccttgcctccacaagcttgagctccggcctctgcctgttcatctacttacctagtactcacagggcagatgggtccaaggctgtgggctgtgatgggcacctggactgcctccttcatcagccaagctcagccctgcatcctattgccctcattccctaaggccagtccccagcagtcccatgggcccacccactcctgtctggatgggttgctcagccctgttctccagctgctcctaactcacggcctttcactgacaaagctggtcgcttgttgcttggcaaggcctgtgctttgcagggagcataaccctggcctgctccgggttgggatgcatagatgaacgtacggacagatacttgagcggtagcaggggtcccagctccctctgttcgtgtcttgcctggctggtgggttcgtgtccctgtgtctgtatgctgtgctgccgtgccacgtctgatgtgttagtgctcggctgccactgttctttcatcaaagccttaacttctgctttatgctcttgtgggaggtgatggggggtggggtcggccagagcagagagggggccagatgctgcccgggagaccagtggggccgagagccccctccctctctctcgggccttccctgccaaactggagaatcctcaccccaaagcatcccgggttcccagcactggcttggccagcacagctggaaagcacatcagggcgggtccattcgtcccctggcgtcaggggcctgagtaagcacatggcagcgtccgcttgcatcgtgtctgttctatgtttttatatttacatctatatatctataattttattaaaaaaaggaaaaatcattttgagcctttccttgggcaaggctgtggctgtgggtacaggggtgtctcaggaaaggggttggccagctggtgcagggtggcatgttcctcttggacagctctgctggcattgccaagtcaggcctagttggtaacgaatggacttctgctggtgagttgcaaaggtgtcttactcccagtctgtgtcatgccggacggaagggtgtttggtgaaaggctaggacctcagctgcctcctgggactctgaggtaccaaagggcttggagcagctatatagtggaggctgcacatgaggccaatgcctggccgagaaacctacagatactactatacttcttccctgccatatgccaagggaggaattagggagtgtcaggaagaactcagagcagggctggaaagggctagggtaggtccagagtttcaagaaggggaggtgtccagagcctggggccccttagttctacccactgctgacttccacctttctgcttctctctcatggatatcggccaacagggggcgcattggcagctgtagccgtggatgggggtggcgtggtacgcctagggggactcctggcctccacctatgccaccttggccaccctgaggatcacccgccgctgcgggccccctggagcgtggctgctggggcagccccccgtgggaccatttgtcggctacattctgctgcatccagccttttccccagcctctcaggtccctaacaggacactagtaggtggctctagggccatggacagtcttctggggacccagaagacgaggttgctgtagagctttggagttgggctggatgtagcctggagatccctaccccgttccccgtcaaccagccacatcttgactgatcagtctgccagggtccacgaccctgtctccaggtgacctcaggaagtccccccacctgctgcaggggtccagtgccccaatgggggtggcctcagagctactgggaaccagcatgacctttaccccaacctccttaccccttccaagcactttaagccacttcctctagggagcccaggcctctgtgggttgggctggggtgggggtctcaggtcaccgcaggttactgcactcctccactcaaccccaagcccctgaacagatgccttaaagtcctggagctgccagccagcttggtgcaccatgaacctggtccacccgtctctgcctcttgcctgctctgacccacctgggtcttctgcagccaagggtgatacctctgggaatacccaagcctgtgggcctggatacccctgggaggagtcagaacagaacccctgccaactcctcttttcacacaggggctgtgtcctgggggctacttctgggcagcagttatgtgtctcccatatggggacagatcacaaactgttttctgttgtataggatttccctccctcaacttccctaataaattttttttttgttcacctccaaaaaaaaaaaaaaaaaaaa

SEQ ID NO: 81 - DNA, 852 bp, synthetic construct
  source 1..852: mol_type = other DNA
ggggggcggagggaagagcggacgggcgggagcgccggcgccagacgcggaggaaaggagctgcgactagccgcccagaggccgccgagccagcgacgcctgagctagtcgagccaccgtcgccgcgcccccatggcggccgccaaggacagtcacgaggaccatgatacttccacagagaatgcagatgagtccaaccacgacccccagttcgagccaatagtttctcttcccgagcaagaaattaaaacgctggaggaagatgaagaggaactttttaagatgcgtgcaaagctgttccggtttgcttcagagaatgacctcccagaatggaaggagcgaggcactggagatgtcaagcttctgaagcacaaggagaaagggaccatccgccttcttatgaggagggacaaaaccttgaagatatgcgccaaccactatattacaccaatgatggagctgaagccgaatgctggcagtgaccgagcctgggtctggaatacccacgccgactttgctgacgagtgccccaagcctgagctgctcgccatccgcttcctaaatgctgagaatgcacaaaagttcaaaacaaagtttgaagaatgcaggaaagaaattgaagagagagaaaagaaaggaccaggcaaaaatgataatgccgaaaaggtggccgagaagctggaagccctttcagtgagggaggccagagaggaggctgaagagaagtctgaggagaaacaatgaatcactctgtctttttcctttccttttctttttaaaaatttgccctaccctttaaggtttgtttttctgttttgtttttacaagggactttataaagaactgaattcc

SEQ ID NO: 82 - DNA, 2155 bp, synthetic construct
  source 1..2155: mol_type = other DNA
tcacccctcagccggcctcggcctccaccgctggtcgccgcgccccgcccgcgcgcccgccgccacacgtcccccgccggcggccaccatgagcacaggactgcggtacaaaagcaagctggcgaccccagaggacaaacaggacatcgacaagcagtacgttggcttcgccacactgcccaaccaggtgcaccgcaagtccgtcaagaaaggtttcgacttcacgctcatggtggccggtgagtccggcctggggaagtccacccttgtccatagcctctttctgaccgacctgtataaggaccggaaactgctgagtgctgaggaacgcatcaaccagacggtagagatcctgaaacacaccgtcgacattgaggagaagggggtcaagttaaagctcaccattgtggacacgcccggctttggggacgcggtgaacaactctgaatgttggaagcccatcactgactatgtggaccagcagtttgagcagtatttccgtgatgagagtggcctgaaccgcaagaacatccaggacaaccgggtacactgctgcctgtacttcatctccccgttcggacacggactgaggccagtggatgtaggcttcatgaaggcactgcatgagaaggtgaacatcgtcccactcatcgccaaagctgactgcctggtgcccagtgagatccggaagctgaaggacagaatacgtgaggagatcgacaagtttgggatccacgtgtaccagtttccagaatgtgattcggatgaagatgaagacttcaagcaacaggaccgggaactgaaggaaagtgcacccttcgccgttattggcagcaacactgtggtggaggccaaggggcagcgggtccgggggcgactgtacccctgggggatcgtcgaagtggagaatcaggcgcactgcgactttgtgaagctccgcaacatgctcatccgcactcacatgcacgacctcaaagatgtgacgtgcgacgtgcactatgagaactaccgtgcccactgcatccagcagatgaccagcaaactcacccaggacagccgcatggagagccccattcctatcctcccactacccacaccggatgcggagaccgagaagctcatcaggatgaaggatgaagagctaaggcgcatgcaggagatgttgcagaagatgaagcagcaaatgcaagaccagtgacacccgccccagccccacgtcgtcgacaaggatagacggccggtttccgggctggcccctcctacccctggatcccagactgtcctggattccaccctgggttcatctggatctcagaaggcctggacctcaccctaatccaaagtggctttgaccagactgttcagacctggagccacagagccacagcccccagatgaccctaatttattctcggcgtccacccttcccggtcatttgtatctgcttccgagtgctctgaatcacagcccctccccaacctcctgcccccgccccaccccgcacctccccgcctatcataagggaacaagatgtgcacaacctccaagatctcccttcctagaagatcactgccccccagcgggccaataaaccagggtagaggagggacgtggatgcaggatcttgggcctattacccaagctagtgctgcagagtggagttgggaggcccccctctgccgattccagtgggctacgaagcatttgctaatggcctactgagcgcggaagtaggccggttcctcccttactccctaatcatgctctgttcaggatcggaggcggcggggtttggaggctaaggcctttaggaagaccccagatctagaggcttctgggaggaaggcggggccgcgatgctctagaccgctcgcgccccttcctgagtctacctgaaggactccaagggcggtagagacggggtgtccctatgcctagactactttcgtccacccataggtcaaggtcttttcttcccagcaactctgttgcacaaggattccagcccttggcctcctccatatctccacccgcatgattcctccccacacaccccatgctccgttttgttcagttgtgaatgccgcgtcctgtcctggtgacaggagaacaaagttggtgaacgtcaaaaaaaaaaaaaaaaaaaaaaaaaaaa

SEQ ID NO: 83 - DNA, 1938 bp, synthetic construct
  source 1..1938: mol_type = other DNA
atcctctcctggcccgcgctgcgagcgccccgccagtccgcgccgccgccctcaccctgtgcgcccgcagcccgcgagcccagcccggcccggtagagcggagcgccggagcctcgtcccgcggccgggccgggaccgggccggagcagcggcgcctggatgcggacccggccgcgcgcagacgggcgcccgccccgaagccgcttccagtgcccgacgcgccccgctcgaccccgaagatgaagagggcgtcctccggaggaagcaggctgctggcatgggtgttatggctacaggcctggagggtagcaacaccatgccctggtgcttgtgtgtgctacaatgagcccaaggtaacaacaagctgcccccagcagggtctgcaggctgtgcccactggcatcccagcctctagccagcgaatcttcctgcatggcaaccgaatctctcacgtgccagctgcgagcttccagtcatgccgaaatctcactatcctgtggctgcactctaatgcgctggctcggatcgatgctgctgccttcactggtctgaccctcctggagcaactagatcttagtgataatgcacagcttcatgtcgtggaccctaccacgttccacggcctgggccacctgcacacactgcacctagaccgatgtggcctgcgggagctgggtcccggcctattccgtggactagcagctctgcagtacctctacctacaagacaacaatctgcaggcactccctgacaacacctttcgagacctgggcaacctcacgcatctctttctgcatggcaaccgtatccccagtgtgcctgagcacgctttccgtggcctgcacagtcttgaccgcctcctcttgcaccagaaccatgtggctcgtgtgcacccacatgccttccgggaccttggccgcctcatgaccctctacctgtttgccaacaacctctccatgctgcctgcagaggtcctaatgcccctgaggtctctgcagtacctgcgactcaatgacaacccctgggtgtgtgactgccgggcacgtccactctgggcctggctgcagaagttccgaggttcctcatcagaggtgccctgcaacctgccccaacgcctggcagaccgtgatcttaagcgcctcgctgccagtgacctagagggctgtgctgtggcttcaggacccttccgtcccatccagaccagtcagctcactgatgaggagctgctgagcctccccaagtgctgccagccagatgctgcagacaaagcctcagtactggaacccgggaggccagcttctgccggaaacgccctcaagggacgtgtgcctcccggtgacactccaccaggcaatggctcaggccctcggcacatcaatgactctccatttggaactttgcccagctctgcagagcccccactgactgccctgcggcctgggggttccgagccaccaggacttcccaccactggtccccgcaggaggccaggttgttcccggaagaatcgcacccgcagccactgccgtctgggccaggcgggaagtggggccagtggaacaggggacgcagagggttcaggggctctgcctgctctggcctgcagccttgctcctctgggccttgcactggtactttggacagtgcttgggccctgctgaccagccaccagccaccaggtgtgtgtacatatggggtctccctccacgccgccagccagagccagggacaggctctgaggggcaggccaggccctccctgacagatgcctccccaccagcccacccccatctccaccccatcatgtttacagggttccgggggtggcgtttgttccagaacgccacctcccacccggatcgcggtatatagagatatgaattttattttacttgtgtaaaatatcggatgacgtggaataaagagctcttttcttaa

SEQ ID NO: 84 - DNA, 34 bp, synthetic construct
  source 1..34: mol_type = other DNA
ctctgatcagcggcgggtggccttcgggtcatga

SEQ ID NO: 85 - DNA, 18 bp, synthetic construct
  source 1..18: mol_type = other DNA
gtggccttcgggtcatga

SEQ ID NO: 86 - DNA, 44 bp, synthetic construct
  source 1..44: mol_type = other DNA
aggcagacgaatgttccccacgttccaagtgaagctttttggaa

SEQ ID NO: 87 - DNA, 18 bp, synthetic construct
  source 1..18: mol_type = other DNA
aggcagacgaatgttccc

SEQ ID NO: 88 - DNA, 44 bp, synthetic construct
  source 1..44: mol_type = other DNA
agggacacggctctggcttcagcccggcagcggccgaacaatga

SEQ ID NO: 89 - DNA, 18 bp, synthetic construct
  source 1..18: mol_type = other DNA
gcagcggccgaacaatga

SEQ ID NO: 90 - DNA, 18 bp, synthetic construct
  source 1..18: mol_type = other DNA
ttgtgatctggaatatgt

SEQ ID NO: 91 - DNA, 44 bp, synthetic construct
  source 1..44: mol_type = other DNA
aaaactctggtcttttaaagtagtcttaactgctcataatatgg

SEQ ID NO: 92 - DNA, 20 bp, synthetic construct
  source 1..20: mol_type = other DNA
cttaactgctcataatatgg

SEQ ID NO: 93 - DNA, 18 bp, synthetic construct
  source 1..18: mol_type = other DNA
ccgcaggagaagcgatga

SEQ ID NO: 94 - DNA, 37 bp, synthetic construct
  source 1..37: mol_type = other DNA
ttaagcctcctgagcgcgtgtatcggctccgccatgg

SEQ ID NO: 95 - DNA, 18 bp, synthetic construct
  source 1..18: mol_type = other DNA
gtatcggctccgccatgg

SEQ ID NO: 96 - DNA, 18 bp, synthetic construct
  source 1..18: mol_type = other DNA
atggctctcaagcgcgtc

SEQ ID NO: 97 - DNA, 44 bp, synthetic construct
  source 1..44: mol_type = other DNA
gcgacgcctgagctagtcgagccaccgtcgccgcgcccccatgg

SEQ ID NO: 98 - DNA, 18 bp, synthetic construct
  source 1..18: mol_type = other DNA
gtcgccgcgcccccatgg

SEQ ID NO: 99 - DNA, 18 bp, synthetic construct
  source 1..18: mol_type = other DNA
atggcggccgccaaggac

SEQ ID NO: 100 - DNA, 44 bp, synthetic construct
  source 1..44: mol_type = other DNA
cgcccggcccggggagggatgcggcggcgcggcgcccaggatgc

SEQ ID NO: 101 - DNA, 18 bp, synthetic construct
  source 1..18: mol_type = other DNA
gcgcggcgcccaggatgc

SEQ ID NO: 102 - DNA, 18 bp, synthetic construct
  source 1..18: mol_type = other DNA
atgccccgcagccccggg

SEQ ID NO: 103 - DNA, 44 bp, synthetic construct
  source 1..44: mol_type = other DNA
gccgcttccagtgcccgacgcgccccgctcgaccccgaagatga

SEQ ID NO: 104 - DNA, 18 bp, synthetic construct
  source 1..18: mol_type = other DNA
gctcgaccccgaagatga

SEQ ID NO: 105 - DNA, 18 bp, synthetic construct
  source 1..18: mol_type = other DNA
atgaagagggcgtcctcc

SEQ ID NO: 106 - DNA, 40 bp, synthetic construct
  source 1..40: mol_type = other DNA
atgctgttggctgctgtctcattgggtctcctgttgctgg

SEQ ID NO: 107 - DNA, 18 bp, synthetic construct
  source 1..18: mol_type = other DNA
atgctgttggctgctgtc

SEQ ID NO: 108 - DNA, 44 bp, synthetic construct
  source 1..44: mol_type = other DNA
ttggtttgagttcgtgcagcagccggtccacaacctgctcatgg

SEQ ID NO: 109 - DNA, 18 bp, synthetic construct
  source 1..18: mol_type = other DNA
tccacaacctgctcatgg

SEQ ID NO: 110 - DNA, 34 bp, synthetic construct
  source 1..34: mol_type = other DNA
tcatgacccgaaggccacccgccgctgatcagag

SEQ ID NO: 111 - DNA, 18 bp, synthetic construct
  source 1..18: mol_type = other DNA
tcatgacccgaaggccac

SEQ ID NO: 112 - DNA, 44 bp, synthetic construct
  source 1..44: mol_type = other DNA
ttccaaaaagcttcacttggaacgtggggaacattcgtctgcct

SEQ ID NO: 113 - DNA, 18 bp, synthetic construct
  source 1..18: mol_type = other DNA
gggaacattcgtctgcct

SEQ ID NO: 114 - DNA, 44 bp, synthetic construct
  source 1..44: mol_type = other DNA
tcattgttcggccgctgccgggctgaagccagagccgtgtccct

SEQ ID NO: 115 - DNA, 18 bp, synthetic construct
  source 1..18: mol_type = other DNA
tcattgttcggccgctgc

SEQ ID NO: 116 - DNA, 18 bp, synthetic construct
  source 1..18: mol_type = other DNA
acatattccagatcacaa

SEQ ID NO: 117 - DNA, 44 bp, synthetic construct
  source 1..44: mol_type = other DNA
ccatattatgagcagttaagactactttaaaagaccagagtttt

SEQ ID NO: 118 - DNA, 20 bp, synthetic construct
  source 1..20: mol_type = other DNA
ccatattatgagcagttaag

SEQ ID NO: 119 - DNA, 18 bp, synthetic construct
  source 1..18: mol_type = other DNA
tcatcgcttctcctgcgg

SEQ ID NO: 120 - DNA, 37 bp, synthetic construct
  source 1..37: mol_type = other DNA
ccatggcggagccgatacacgcgctcaggaggcttaa

SEQ ID NO: 121 - DNA, 18 bp, synthetic construct
  source 1..18: mol_type = other DNA
ccatggcggagccgatac

SEQ ID NO: 122 - DNA, 18 bp, synthetic construct
  source 1..18: mol_type = other DNA
gacgcgcttgagagccat

SEQ ID NO: 123 - DNA, 44 bp, synthetic construct
  source 1..44: mol_type = other DNA
ccatgggggcgcggcgacggtggctcgactagctcaggcgtcgc

SEQ ID NO: 124 - DNA, 18 bp, synthetic construct
  source 1..18: mol_type = other DNA
ccatgggggcgcggcgac

SEQ ID NO: 125 - DNA, 18 bp, synthetic construct
  source 1..18: mol_type = other DNA
gtccttggcggccgccat

SEQ ID NO: 126 - DNA, 44 bp, synthetic construct
  source 1..44: mol_type = other DNA
gcatcctgggcgccgcgccgccgcatccctccccgggccgggcg

SEQ ID NO: 127 - DNA, 18 bp, synthetic construct
  source 1..18: mol_type = other DNA
gcatcctgggcgccgcgc

SEQ ID NO: 128 - DNA, 18 bp, synthetic construct
  source 1..18: mol_type = other DNA
cccggggctgcggggcat

SEQ ID NO: 129 - DNA, 44 bp, synthetic construct
  source 1..44: mol_type = other DNA
tcatcttcggggtcgagcggggcgcgtcgggcactggaagcggc

SEQ ID NO: 130 - DNA, 18 bp, synthetic construct
  source 1..18: mol_type = other DNA
tcatcttcggggtcgagc

SEQ ID NO: 131 - DNA, 18 bp, synthetic construct
  source 1..18: mol_type = other DNA
ggaggacgccctcttcat

SEQ ID NO: 132 - DNA, 40 bp, synthetic construct
  source 1..40: mol_type = other DNA
ccagcaacaggagacccaatgagacagcagccaacagcat

SEQ ID NO: 133 - DNA, 18 bp, synthetic construct
  source 1..18: mol_type = other DNA
gacagcagccaacagcat

SEQ ID NO: 134 - DNA, 44 bp, synthetic construct
  source 1..44: mol_type = other DNA
ccatgagcaggttgtggaccggctgctgcacgaactcaaaccaa

SEQ ID NO: 135 - DNA, 18 bp, synthetic construct
  source 1..18: mol_type = other DNA
ccatgagcaggttgtgga

SEQ ID NO: 136 - DNA, 43 bp, synthetic construct
  source 1..43: mol_type = other DNA
gatgcggccgccactgtgctggatatctgcagaattcgccctt